(19) United States
(12) Patent Application Publication
(10) Pub. No.: US 2021/0369650 A1
(43) Pub. Date: Dec. 2, 2021

(54) TREATMENT OF INFLAMMATORY CONDITIONS

(71) Applicant: UNION therapeutics A/S, Hellerup (DK)

(72) Inventors: Emma GUTTMAN-YASSKY, New York, NY (US); Morten Otto Alexander SOMMER, Hellerup (DK); Rasmus TOFT-KEHLER, Hellerup (DK); Anne WEISS, Hellerup (DK); Ana Brandusa PAVEL, New York, NY (US)

(21) Appl. No.: 17/290,386

(22) PCT Filed: Nov. 2, 2018
(86) PCT No.: PCT/US2018/058918

§ 371 Date: Apr. 30, 2021

(51) Int. Cl. A61K 31/167; A61K 47/10; A61K 9/00; A61P 17/00

(52) U.S. Cl. CPC A61K 31/167; A61P 17/00; A61K 9/0014; A61K 47/10

(57) ABSTRACT

The invention relates to a halogenated salicylanilide selected from niclosamide and oxyclozanide, or a pharmaceutically acceptable salt or hydrate thereof, for use in topical anti-inflammatory treatment of one or more clinical signs or symptoms of an inflammatory skin condition in a subject, wherein the one or more clinical signs or symptoms are associated with an abnormal inflammatory response, and the topical anti-inflammatory treatment modulates expression in lesional skin of one or more immune effectors selected from proinflammatory mediators and skin barrier molecules.

This invention relates to a halogenated salicylanilide selected from niclosamide and oxyclozanide (particularly niclosamide), or a pharmaceutically acceptable salt or hydrate thereof, for use in the topical anti-inflammatory treatment of one or more clinical signs or symptoms of an inflammatory skin condition, such as atopic dermatitis (AD), in a subject.

The invention also relates to a halogenated salicylanilide selected from niclosamide and oxyclozanide (particularly niclosamide), or a pharmaceutically acceptable salt or hydrate thereof, for use in the topical anti-inflammatory treatment of one or more clinical signs or symptoms of an inflammatory ocular condition, such as dry eye disease (DED), in a subject.

### BACKGROUND

Inflammation is the response of body tissue to infections, irritation or injury. Heat, pain, redness and swelling are common signs of inflammation.

The immune system protects the host from pathogens and is regulated by interaction of the innate immune system and the adaptive immune system. The complement system, which is part of the innate immune system, provides a functional bridge between innate and adaptive immune responses.

The innate immunity (also called natural or native immunity) is non-specific and the initial defence against pathogens, such as bacteria, virus and fungi. The skin innate immune system includes three major components: (i) anatomical/physical barrier, including the stratum corneum (SC), the outermost layer of the epidermis which comprises lipids (e.g. ceramides, cholesterol, and fatty acids), keratins, cornified envelope proteins (e.g. loricrin, filaggrin, periplakin and “small proline-rich” proteins (SPRR)), and intracellular junctions formed by proteins called cadherins; (ii) cellular, including macrophages, Langerhans cells, dendritic cells, keratinocytes (KCs), natural killer (NK) cells, mast cells, and polymorphonuclear neutrophils (PMNs)); and (iii) secretory mediators, including antimicrobial peptides and proteins (AMPs), proinflammatory enzymes, and cytokines (e.g. chemokines) (Benedetto et al, Journal of Investigative Dermatology, vol 129, issue 1, pages 14-30, 2009).

The adaptive immunity (also called the acquired immunity) is highly specific to a particular pathogen (antigen) and provides an antigen-specific response. The acquired immune system is cell mediated and acts through T and B lymphocytes. Activated B lymphocytes secrete antibodies that neutralize the pathogen and activated cytotoxic T lymphocytes cells (“killer” T cells) induce the death of cells that are infected with pathogens. “Helper” T cells are immune response mediators which have no cytotoxic or phagocytic activity. For example, T-helper 2 (Th2) cells secrete cytokines that drive the type-2 pathway (“humoral immunity”) which induces B cells to produce antibodies against the antigen.

Innate and adaptive immune systems converge into three major kinds of cell-mediated effector immunity; type 1, type 2, and type 3. Type 1 immunity involves activation of Th1 cells and is also referred to as Th1-type immune response. Type 2 immunity involves activation of Th2 cells and is also referred to as Th2-type immune response. Type 3 immunity involves activation of Th17 cells and is also referred to as Th17-type immune response. Type 1 and Type 3 immunity mediate autoimmune diseases, whereas type 2 responses can be involved in allergic diseases (Annunziato et al, The Journal of Allergy and Clinical Immunology, vol 135, issue 3, pages 626-635, 2015).

A disorder in any part of the skin defence system can induce an inflammatory skin condition. Psoriasis, rosacea and atopic dermatitis are examples of inflammatory skin conditions which are believed to be associated with an inherent dysfunction of the innate immune system (Gallo et al, Drug Discov. Today Dis. Mech. 5(2):145-152, 2008). Cathelicidin dysfunction have been found in psoriasis, rosacea and atopic dermatitis patients. In atopic dermatitis patients, cathelicidin is suppressed (Schauber et al, J Allergy Clin Immunol., 122(2):262-266, 2008).

Guttman-Yassky et al (“Contrasting pathogenesis of atopic dermatitis and psoriasis—part I: Clinical and pathological concepts”, The Journal of Allergy and Clinical Immunology, vol. 127, issue 5, pages 1110-1118, 2011; and “Contrasting pathogenesis of atopic dermatitis and psoriasis—part II: Immune cell subsets and therapeutic concepts”, The Journal of Allergy and Clinical Immunology, vol. 127, issue 6, pages 1420-1432, 2011) review similarities and differences between atopic dermatitis and psoriasis in epidermal barrier defects and immune cells. Guttman-Yassky et al stress that the diseases share barrier abnormalities, but there are differences between the diseases. AD skin lesions have reduced terminal differentiation of keratinocytes, decreased cornification (including decreased expression of filiaggrin and loricrin), and reduced levels of lipids. In contrast, psoriatic lesions have increased cornification and expression of terminal differentiation proteins, with reduced lipids in the intracellular spaces of the stratum corneum.

Psoriasis is a chronic inflammatory skin condition which causes cells to build up rapidly on the surface of the skin. These plaques of extra skin cells are red, dry, itchy, scaly and sometimes painful. Psoriasis is believed to be caused by a variety of factors in including host genetics and environmental factors. There are several types of psoriasis, such as plaque psoriasis (also called psoriasis vulgaris), guttate psoriasis, inverse psoriasis, pustular psoriasis and erythrodermic psoriasis. Psoriasis is considered to be mediated by the innate immune system as well as the adaptive immune system (Buchau et al, Clin. Dermatol., 25(6): 616-624, 2007). Psoriasis is normally treated with steroid cream, vitamin D3 cream, ultraviolet light or immune system suppressing medications, such as methotrexate. Biologics (e.g. adalimumab and brodalumab) are also frequently used including antibodies targeting inflammatory mediators such as TNF alpha and IL17A.

Rosacea is an inflammatory skin disease affecting primarily cheeks, nose chin and forehead. Rosacea results in redness, pimples, swelling and small and superficial dilated blood vessels. Current treatment of rosacea includes antibiotics, such as metronidazole, ivermectin, doxycycline or tetracycline. It has been suggested that an altered innate immune response is involved in rosacea (Yamasaki, et al, Journal of Investigative Dermatology Symposium proceedings, 15(1):12-5, 2011. Patients with rosacea display overexpression of inflammatory mediators (REF) (Buhl, Timo, et al. “Molecular and morphological characterization of inflammatory infiltrate in rosacea reveals activation of Th1/Th17 pathways.” Journal of Investigative Dermatology 135.9 (2015): 2198-2208).

Dermatitis (also referred to as eczema) is an inflammatory skin condition characterized by one or more of erythema, pruritus, scaling, oozing, crusting and vesicles. There are numerous forms of dermatitis, with atopic dermatitis being the most common.

Atopic dermatitis (AD) is an inflammatory condition of the skin characterized by erythema, pruritus, scaling, lichenification, and papulovesicles. AD often develops in early childhood and is estimated to affect 15 to 20% of children and 1-3% of adults (Leung et al. J. Allergy Clin. Immunol. 2014; 134(4):769-79 and Weidinger et al. Lancet. 2016; 387(10023):1109-22).

AD is a complex condition associated with an impaired innate immune response in which the skin barrier at the site of lesions is compromised enabling triggers such as irritants, allergens, dust mites, bacteria and/or foods to penetrate the skin and initiate an inflammatory reaction. Maintz et al (“Modifications of the Innate Immune System in Atopic Dermititis”, J Innate Immune, 3: 131-141, 2011) provides an overview of the innate immune system in AD. The inflammatory response in atopic dermatitis is thought to be mediated predominantly by Th2 (Bieber T. Atopic dermatitis. N. Engl. J. Med. 2008; 358(14):1483-94). Brunner et al (The Journal of Allergy and Clinical Immunology, Volume 139, Issue 4, Supplement, Pages S65-S76, 2017) stress that atopic dermatitis, in contrast to psoriasis, is a much more heterogeneous disease and discuss the immune map of atopic dermatitis patients with activation of predominantly Th2 but also the Th22, Th17 and Th1 signalling pathways depending on the subtype of the disease.

Symptoms of AD include patches of skin that are red or brownish, dry, cracked or scaly. A particularly problematic symptom of AD is pruritus (itchy skin), which can have a significant effect on a patients' quality of life including sleep deprivation, social stigmatisation and psychiatric effects including depression and anxiety (Kafferman G, et al. 2014. Sleep Medicine Reviews; 14:359-369; Erturk et al Ann. Dermatol. 2012 November; 24(4): 406-412).

As discussed above, the innate immunity defence includes antimicrobial proteins and peptides (AMPs) in the skin barrier. These AMPs have been found to be downregulated in AD patients while being abundant in psoriatic lesions (Buchau et al, Clin. Dermatol., 25(6): 616-624, 2007).

The compromised barrier function of the skin including the deficiency in expression of AMPs in skin lesions of patients with atopic dermatitis results in dermatitis lesions being prone to bacterial infection, particularly by Staphylococcus aureus. Lesion colonization by S. aureus is a significant factor in the pathogenesis of atopic dermatitis for recurrent complications that exacerbate the disorder. Its presence, even without overt infection, appears to trigger multiple inflammatory reactions, via toxins, that act as super antigens and exogenous protease inhibitors that further damage the epidermal barrier and potentiate allergen penetration. (Bieber T. Atopic dermatitis. N. Engl. J. Med. 2008; 358(14):1483-94). It has been shown that patients with atopic dermatitis have a decreased skin bacterial diversity during the recurrent flaring of intensely itchy skin. Particularly, an increase of Staphylococcus aureus colonization has been observed during flares (Salava et al, Role of the skin microbiome in atopic dermatitis, Clin Transl Allergy, 4:33, 2014).

Mansouri et al (J Clin Med, 4(5): 858-873, 2015) identified biomarkers for assessment of effective treatment of atopic dermatitis. These biomarkers include measures of epidermal hyperplasia (epidermal thickness, and protein and mRNA expressions of KRT16 and Ki67), S100 responses, T-cell (CD3+ and CD8+) and DC (i.e., CD83+, CD206, FceRI+, etc) cellular infiltrates, and cytokines and chemokines defining Th2-, Th22-, and Th17-activation (including IL13, IL22, IL19, MMP12, S100A12, CCL17, CCL18, CCL22, elafin/PI3, IL23A, etc) with modest effects on terminal differentiation genes (e.g., LOR and FLG).

Current treatments for dermatitis such as AD typically target one or more symptoms of the dermatitis and include, the use of skin emollients (e.g. moisturisers and oils) to moisturise the skin, topical corticosteroids, anti-histamines to relieve itching and antibiotics including clindamycin, dicloxacillin, first-generation cephalosporins and macrolide antibiotics to treat secondary infections of skin lesions. Patients may also be treated with an immunosuppressant such as cyclosporin, tacrolimus or azathioprine. Phototherapy is also employed as a second-line treatment after failure of first-line treatments (Sidbury et al. Guidelines of care for the management of atopic dermatitis: section 3. J Am Acad Dermatol. 2014 August; 71(2):327-49).

Topical corticosteroids can be effective in reducing inflammation and certain other symptoms of dermatitis, such as AD. However, the chronic use of topical corticosteroids is associated with undesirable side-effects, particularly skin atrophy.

Recently, dupilumab, was approved by the FDA for the treatment of adult patients with moderate-to-severe atopic dermatitis whose disease is not adequately controlled with topical prescription therapies. Dupiliumab, inhibits interleukin-4 and interleukin-13 signalling by binding to interleukin-4 receptor a.

The nonsteroidal phosphodiesterase 4 (PDE4) inhibitor crisaborole ointment was approved by the FDA in 2016 for the topical treatment of mild to moderate atopic dermatitis (AD) in patients two years of age and older.

There remains a need for new treatments for inflammatory skin conditions, particularly inflammatory skin conditions associated with an impaired immune response, including inflammatory skin conditions associated with abnormal inflammatory response and skin barrier dysfunction, such as psoriasis, rosacea and atopic dermatitis.

Dry eye disease (DED), also called keratoconjunctivitis sicca, is a common inflammatory ocular disease. Dry eye has been shown to be associated with abnormalities in the pre-corneal tear film and subsequent inflammatory changes in the entire ocular surface including the adnexa, conjunctiva and cornea (Hessen et al, J Ophthalmic Vis Res, 9(2): 240-250, 2014). Current medications include cyclosporine A, corticosteroids (e.g. dexamethasone), tacrolimus, tetracycline derivatives and autologous serum. A lot of these presently used anti-inflammatory agents such as cyclosporin can cause irritation in the patient's eye.

Jabs et al, “Guidelines for the Use of Immunosuppressive Drugs in Patients with Ocular Inflammatory Disorders: Recommendations of an Expert Panel”, Am J Ophthalmol, 130(4): 492-513, 2000, provide recommendations for the use of immunosuppressive drugs in the treatment of patients with ocular inflammatory disorders.

There is a need also for new treatments for inflammatory ocular conditions associated with abnormal inflammatory response and pre-corneal tear film dysfunction, such as dry eye disease (DED) (also called dry eye disorder or dry eye syndrome).

Halogenated salicylanilides such as oxyclozanide and niclosamide are anthelmintic drugs.

Oxyclozanide is used in veterinary medicine, primarily for ruminants, such as cattle, goat and sheep, to control parasitic flatworms called flukes.

Niclosamide is approved for use as an anthelmintic drug for human and veterinary medicine. Niclosamide is a known taenicide effective against several parasitic tapeworms of livestock and pets (e.g. Taenia spp, Moniezia spp) and also against rumen flukes (Paramphistomum spp) and blood flukes (Schistosoma spp.). Niclosamide has also been shown to prevent the penetration of Schistosoma mansoni through the human skin. As well as used as an anticancer drug, pesticide and as an anti-trypanosoma drug. Niclosamide has also been shown to inhibit viral replication in human cells. (Ofori-Adjei et al; The International Journal of Risk & Safety in Medicine. 2008; 20:113-22; and Pearson et al; Annals of Internal Medicine. 1985; 102(4):550-1).

GB 2,456,376 and WO 2008/155535 describes the use of halogenated salicylanilides for the treatment of acne caused by propionibacteria.

WO 2016/038035 discloses the use of halogenated salicylanilides for the topical treatment of diseases or infections caused by Gram-positive bacteria. The use of niclosamide for topical prevention and/or treatment of Gram-positive bacteria such as Staphylococcus aureus (S. aureus) and/or Streptococcus pyogenes (S. pyogenes) colonizing or infecting skin affected with a dermatological condition selected from the group consisting of impetigo, atopic dermatitis and infections associated with different skin conditions such as eczema or psoriasis is disclosed.

WO 2017/157997 A1 discloses non-aqueous topical compositions comprising a halogenated salicylanilide, such as niclosamide, and the use of such compositions in the topical treatment or prevention of an infection or disease caused by Gram-positive bacteria.

Wu et al. (Cellular Immunity, 288 (2014) 15-23) discloses that niclosamide has an inhibitory action on lipopolysaccharide (LPS)-induced dendritic cell maturation and cytokine costimulatory molecule and MHC molecule expression in-vitro. It was also found that niclosamide-treated dendritic cells inhibited antigen specific T cell responses. The reference postulates that niclosamide may be useful for the treatment of chronic inflammatory disorders or dendritic cell mediated autoimmune disease, however, no clinical data is provided, and the conclusions of the paper indicate that further studies are required to better understand the molecular mechanisms associated with the compound.

U.S. Pat. No. 4,742,083 suggests the use of certain substituted salicylamides as systemic analgesic agents and also for topical application as anti-inflammatory compositions.

WO 2017/040864 suggest the use of a compound exhibiting activity as a mitochondrial uncoupling agent, for example niclosamide, for treating one or more symptoms of a pathology characterized by an abnormal inflammatory response (e.g. inflammatory bowel disease) in a subject. The disclosure is said to be based on the finding that niclosamide kills lamina propria T cells (expressing CD3) isolated from IBD patients. Methods for inducing cell death of one or more T cells and thereby treat a condition, or one or more symptoms thereof, selected from the group consisting of celiac disease, irritable bowel syndrome, mucositis, uveitis, collagenous colitis, lymphocytic colitis, microscopic colitis, radiation enteritis, rheumatoid arthritis, lupus, scleroderma, psoriasis, cutaneous T-cell lymphoma, acute graft vs. host disease and chronic graft vs. host disease by topically and locally administering the compound to the subject are suggested.

### BRIEF SUMMARY OF THE DISCLOSURE

Niclosamide has been found to have activity as topical immunomodulator in epithelial tissue, such as lesional skin. Particularly, topical application of niclosamide to epithelial tissue, such as lesional skin, has been found to provide an immunosuppressive activity in epithelial tissue, such as lesional skin. More particularly, topical application of niclosamide to epithelial tissue, such as lesional skin, has been found to provide an immunosuppressive activity on some proinflammatory mediators and an immunopromotive activity on some barrier molecules in epithelial tissue, such as skin barrier molecules in lesional skin.

In a first aspect of the invention, there is provided a halogenated salicylanilide selected from niclosamide and oxyclozanide (particularly niclosamide), or a pharmaceutically acceptable salt or hydrate thereof, for use in topical anti-inflammatory treatment of one or more clinical signs or symptoms of an inflammatory skin condition in a subject, wherein the one or more clinical signs or symptoms are associated with an abnormal inflammatory response, and the topical anti-inflammatory treatment modulates expression in lesional skin of one or more immune effectors selected from proinflammatory mediators and skin barrier molecules. The treatment may reduce or eliminate one or more of the clinical signs or symptoms. Particularly, the topical anti-inflammatory treatment modulates endogenous expression in lesional skin of one or more immune effectors selected from proinflammatory mediators and skin barrier molecules.

In embodiments, the one or more clinical signs or symptoms are associated with skin barrier dysfunction, such as skin barrier dysfunction including deficiency in one or more skin barrier molecules, particularly deficiency in one or more structural skin barrier proteins (e.g. FLG or LOR). The treatment may reduce or eliminate one or more of the clinical signs or symptoms associated with the abnormal inflammatory response and the skin barrier dysfunction.

In embodiments, the topical anti-inflammatory treatment decreases (downregulates) expression in lesional skin of one or more proinflammatory mediators.

In embodiments, the topical anti-inflammatory treatment increases (upregulates) expression in lesional skin of one or more skin barrier molecules.

In embodiments, the topical anti-inflammatory treatment decreases expression in lesional skin of one or more proinflammatory mediators and increases expression in lesional skin of one or more skin barrier molecules.

In embodiments, the one or more clinical signs or symptoms are selected from erythema, excoriation, lichenification, edema, papulation, dryness, pruritus, scaling, oozing and crusting.

In embodiments, the one or more clinical signs or symptoms is selected from erythema, excoriation, lichenification, edema, papulation and dryness, particularly the one or more clinical signs or symptoms is selected from erythema, lichenification, edema and papulation.

In embodiments of the invention, the one or more clinical signs or symptoms are associated with an abnormal (such as elevated) level of one or more proinflammatory mediators and the topical anti-inflammatory treatment reduces the abnormal level of said one or more proinflammatory mediators.

In embodiments of the invention, the one or more clinical signs or symptoms are associated with skin barrier dysfunction (e.g. skin barrier deficiency in one or more skin barrier molecules), and the topical anti-inflammatory treatment provides increased (upregulated) expression in lesional skin of one or more skin barrier molecules.

In a second aspect of the invention, there is provided a halogenated salicylanilide selected from niclosamide and oxyclozanide (particularly niclosamide), or a pharmaceutically acceptable salt or hydrate thereof, for use in modulating (such as attenuating) an abnormal inflammatory response associated with an inflammatory skin condition in a subject by topically applying the halogenated salicylanilide selected from niclosamide and oxyclozanide (particularly niclosamide), or a pharmaceutically acceptable salt or hydrate thereof, to the skin of the subject, wherein the abnormal inflammatory response is modulated by modulating expression in lesional skin of one or more immune effectors selected from proinflammatory mediators and skin barrier molecules, thereby decreasing (downregulating) expression in lesional skin of one or more proinflammatory mediators, and increasing (upregulating) expression in lesional skin of one or more skin barrier molecules.

Thus, there is provided a halogenated salicylanilide selected from niclosamide and oxyclozanide (particularly niclosamide), or a pharmaceutically acceptable salt thereof or hydrate thereof, for use in topical treatment of an inflammatory skin condition in a subject by modulating (such as attenuating) an abnormal inflammatory response associated with the inflammatory skin condition, wherein the topical treatment modulates expression in lesional skin of one or more immune effectors selected from proinflammatory mediators and skin barrier molecules, thereby providing decreased (downregulated) expression in lesional skin of one or more proinflammatory mediators and increased (upregulated) expression in lesional skin of one or more skin barrier molecules.

In a third aspect of the invention, there is provided a halogenated salicylanilide selected from niclosamide and oxyclozanide (particularly niclosamide), or a pharmaceutically acceptable salt or hydrate thereof, for use in improving skin barrier function associated with an inflammatory skin condition in a subject by topically applying the halogenated salicylanilide selected from niclosamide and oxyclozanide (particularly niclosamide), or a pharmaceutically acceptable salt or hydrate thereof, to the skin of the subject, wherein the skin barrier function is improved by modulating expression in lesional skin of one or more immune effectors selected from proinflammatory mediators and skin barrier molecules.

In embodiments, the topical anti-inflammatory treatment provides decreased (downregulated) expression in lesional skin of one or more proinflammatory mediators.

In embodiments, the topical anti-inflammatory treatment provides increased (upregulated) expression in lesional skin of one or more skin barrier molecules.

In embodiments, the skin barrier function is improved by modulating expression in lesional skin of immune effectors selected from proinflammatory mediators and skin barrier molecules thereby providing decreased expression in lesional skin of one or more proinflammatory mediators and increased expression in lesional skin of one or more skin barrier molecules.

Thus, there is provided a halogenated salicylanilide selected from niclosamide and oxyclozanide (particularly niclosamide), or a pharmaceutically acceptable salt or hydrate thereof, for use in topical treatment of an inflammatory skin condition in a subject by improving skin barrier function associated with the inflammatory skin condition, wherein the topical treatment modulates expression in lesional skin of one or more immune effectors selected from proinflammatory mediators and skin barrier molecules, such as providing decreased (downregulated) expression in lesional skin of one or more proinflammatory mediators and increased (upregulated) expression in lesional skin of one or more skin barrier molecules.

It has been found that that topical administration of niclosamide, or a pharmaceutically acceptable salt or hydrate thereof, to the skin of a subject for treatment of an inflammatory skin condition in the subject, may reduce or eliminate one or more of clinical signs or symptoms of the inflammatory skin condition by modulating (such as attenuating) an abnormal inflammatory response associated with the clinical signs or symptoms and by improving skin barrier function associated with an inflammatory skin condition. Topical administration of niclosamide, or a pharmaceutically acceptable salt or hydrate thereof, has been found to modulate expression in lesional skin of proinflammatory mediators and skin barrier molecules.

When reference hereinafter is made to the invention, all aspects and embodiments thereof are referred to.

In embodiments of the invention, the one or more immune effectors may be innate immune effectors and/or adaptive immune effectors.

In embodiments of the invention, the one or more immune effectors may be innate immune effectors.

In embodiments of the invention, the one or more immune effectors are one or more proinflammatory mediators.

In embodiments of the invention, the one or more proinflammatory mediators are proinflammatory mediators selected from cytokines, enzymes, antibacterial proteins and peptides, and immune cells.

In embodiments of the invention, the proinflammatory mediators are one or more proinflammatory mediators selected from proinflammatory cytokines, proinflammatory enzymes, antibacterial proteins and peptides, and immune cells.

In embodiments of the invention, the one or more immune effectors are one or more proinflammatory cytokines.

In embodiments of the invention, the one or more immune effectors are one or more proinflammatory enzymes.

In embodiments of the invention, the one or more immune effectors are one or more proinflammatory antibacterial proteins and peptides.

In embodiments of the invention, the one or more immune effectors are one or more proinflammatory immune cells.

In embodiments of the invention, the one or more immune effectors are innate immune effectors selected from proinflammatory innate cytokines, proinflammatory innate enzymes, innate antibacterial proteins and peptides (such as proinflammatory innate antibacterial proteins and peptides), proinflammatory innate immune cells and skin barrier molecules.

In embodiments of the invention, the one or more immune effectors are one or more skin barrier molecules.

In embodiments of the invention, the skin barrier molecules are one or more skin barrier molecules selected from skin barrier proteins, skin barrier peptides and skin barrier lipids.

In embodiments of the invention, the skin barrier molecules are selected from structural skin barrier proteins (e.g. LOR, FLG, DGAT2, FAXDC2) and skin barrier lipids (e.g. ACOX2, EVOLV3, FA2H, FAR2, KRT79, PNPLA3).

In embodiments of the invention, the skin barrier molecules are one or more structural skin barrier lipids (e.g. ACOX2, EVOLV3, FA2H, FAR2, KRT79, PNPLA3).

In embodiments of the invention, the skin barrier molecules are one or more structural skin barrier proteins (e.g. LOR, FLG, DGAT2, FAXDC2).

In embodiments of the invention, the skin barrier molecules are loricrin (LOR) and/or filaggrin (FLG).

In embodiments of the invention, the skin barrier molecules are loricrin (LOR).

In embodiments of the invention, the abnormal inflammatory response involves a Th1, Th2, Th17 and/or Th22-type inflammatory response.

In embodiments of the invention, the abnormal inflammatory response involves a Th2-type inflammatory response.

In embodiments of the invention, the abnormal inflammatory response involves (i) Th2-type inflammatory response, and (ii) Th1, Th17 and/or Th22-type inflammatory response(s).

In embodiments of the invention, the expression of one or more proinflammatory mediators may be associated with activation of Th1, Th2, Th17 and/or Th22 cells.

In embodiments of the invention, the expression of the one or more immune effectors is modulated by attenuating one or more responses selected from of Th1, Th17 and Th22-type inflammatory response(s).

In embodiments of the invention, the expression of the one or more immune effectors is modulated by attenuating Th2-type inflammatory response.

In embodiments of the invention, the expression of the one or more immune effectors is modulated by attenuating (i) Th2-type inflammatory response and one or more response selected from Th1, Th17 and Th22-type inflammatory response(s).

In embodiments of the invention, the one or more immune effectors are selected from S100A12, S100A9, PI3, CXCL1, KRT16, MMP12, IL13, CCL17, CCL22, IL8, S100A8, S100A7, IL22, IL17A, IL19, CAMP, DEFB4A/DEFB4B, LOR, IL1B, IL6, IL17C, IL15, IL15RA, FOXP3, FLG, CXCL10, CCL20, CXCL2, IL12B, IL23A, CCL18, IL10, IL5 and TSLPR. In the study of Example 2, all these biomarkers were found to have changed significantly compared to baseline in the qRT-PCR analysis (Table 9).

In embodiments of the invention, the one or more immune effectors are selected from S100A12, S100A9, PI3, CXCL1, MMP12, IL13, CCL17, CCL22, IL8, S100A8, S100A7, IL22, IL17A, IL19, CAMP, DEFB4A/DEFB4B, IL1B, IL6, IL17C, IL15, IL15RA, FOXP3, CXCL10, CCL20, CXCL2, IL12B, IL23A, CCL18, IL10, IL5 and TSLPR. In the study of Example 2, all these proinflammatory mediators were found to have changed significantly compared to baseline in the qRT-PCR analysis (Table 9).

In embodiments of the invention, the one or more immune effectors are selected from LOR, FLG and KRT16. In the study of Example 2, all these skin barrier molecules were found to have changed significantly compared to baseline in the qRT-PCR analysis (Table 9).

In embodiments of the invention, the one or more immune effectors are selected from LOR and FLG. In the study of Example 2, all these skin barrier molecules were found to have increased significantly compared to baseline in the qRT-PCR analysis (Table 9).

In embodiments of the invention, the one or more immune effectors are selected from S100A9, S100A7, IL17C, CCL20, CCL18 and IL10. In the study of Example 2, all these biomarkers were found to have changed significantly compared to baseline and vehicle in the qRT-PCR analysis (Table 11).

In embodiments of the invention, the one or more immune effectors are selected from CD86, CCL19, IL24, MMP12, ANXA6, SPTLC3, CCR7, CD2, CD28, CD3D, CD3G, CCL2, CCR1, CCR2, IFNGR2, IL12RB2, IL2RA, IRF1, CCR6, CXCL1, CXCL2, IL6R, LCN2, PI3, STAT3, IL37, TNFSF4, S100A12, S100A7, S100A8, S100A9, S100P, SERPINB1, SERPINB4, CCL13, CCL18, CCL22, CCR5, IL4R, IL7R, IL1F10, CDSN, CERS3, CLN8, ELOVL3, EREG, FA2H, FAR2, KRT79, PNPLA3, PPL, TJP3, ACER1, ANXA9, CLDN1, CLDN23, DGAT2, DHCR7, FAXDC2, FLG, KRT23, KRT77, ACOX2, ACSL1 and SCEL. In the study of Example 2, all these biomarkers were found to have changed significantly compared to baseline in the microarray analysis (Table 14).

In embodiments of the invention, the one or more immune effectors are selected from CD86, CCL19, IL24, MMP12, CCR7, CD2, CD28, CD3D, CD3G, CCL2, CCR1, CCR2, IFNGR2, IL12RB2, IL2RA, IRF1, CCR6, CXCL1, CXCL2, IL6R, LCN2, PI3, STAT3, IL37, TNFSF4, S100A12, S100A7, S100A8, S100A9, S100P, SERPINB1, SERPINB4, CCL13, CCL18, CCL22, CCR5, IL4R, IL7R and IL1F10. In the study of Example 2, all these proinflammatory mediators were found to have changed significantly compared to baseline in the microarray analysis (Table 14).

In embodiments of the invention, the one or more immune effectors are selected from ANXA6, SPTLC3, CDSN, CERS3, CLN8, ELOVL3, EREG, FA2H, FAR2, KRT79, PNPLA3, PPL, TJP3, ACER1, ANXA9, CLDN1, CLDN23, DGAT2, DHCR7, FAXDC2, FLG, KRT23, KRT77, ACOX2, ACSL1 and SCEL. In the study of Example 2, all these skin barrier molecules were found to have changed significantly compared to baseline in the microarray analysis (Table 14).

In embodiments of the invention, the one or more immune effectors are selected from CDSN, CERS3, CLN8, ELOVL3, EREG, FA2H, FAR2, KRT79, PNPLA3, PPL, TJP3, ACER1, ANXA9, CLDN1, CLDN23, DGAT2, DHCR7, FAXDC2, FLG, KRT23, KRT77, ACOX2, ACSL1 and SCEL. In the study of Example 2, all these skin barrier molecules were found to have increased significantly compared to baseline in the microarray analysis (Table 14).

In embodiments of the invention, the one or more immune effectors are selected from SPTLC3, CCL2, CCR1, IFNGR2, CCR6, LCN2, STAT3, TNFSF4, CCL13, IL4R, IL1F10, CXCL1, PI3, S100A12, S100A9, CCL18, ELOVL3, FA2H, FAR2, KRT79, PNPLA3, DGAT2, FAXDC2 and ACOX2. In the study of Example 2, all these biomarkers were found to have changed significantly compared to baseline and vehicle in the microarray analysis (Table 16).

In embodiments of the invention, the one or more immune effectors are selected from SPTLC3, CCL2, CCR1, IFNGR2, CCR6, CXCL1, LCN2, PI3, STAT3, TNFSF4, S100A12, S100A9, CCL13, CCL18. IL4R, and IL1F10. In the study of Example 2, all these proinflammatory mediators were found to have changed significantly compared to baseline and vehicle in the microarray analysis (Table 16).

In embodiments of the invention, the one or more immune effectors are selected from ELOVL3, FA2H, FAR2, KRT79, PNPLA3, DGAT2, FAXDC2 and ACOX2. In the study of Example 2, all these skin barrier molecules were found to have changed significantly compared to baseline and vehicle in the microarray analysis (Table 16).

In embodiments of the invention, the one or more immune effectors are selected from S100A12, S100A9, PI3, CXCL1, KRT16, MMP12, IL13, CCL17, CCL22, IL8, S100A8, S100A7, IL22, IL17A, IL19, CAMP, DEFB4A/DEFB4B, LOR, IL1B, IL6, IL17C, IL15, IL15RA, FOXP3, FLG, CXCL10, CCL20, CXCL2, IL12B, IL23A, CCL18, IL10, IL5, TSLPR, CD86, CCL19, IL24, ANXA6, SPTLC3, CCR7, CD2, CD28, CD3D, CD3G, CCL2, CCR1, CCR2, IFNGR2, IL12RB2, IL2RA, IRF1, CCR6, IL6R, LCN2, STAT3, IL37, TNFSF4, S100P, SERPINB1, SERPINB4, CCL13, CCR5, IL4R, IL7R, IL1F10, CDSN, CERS3, CLN8, ELOVL3, EREG, FA2H, FAR2, KRT79, PNPLA3, PPL, TJP3, ACER1, ANXA9, CLDN1, CLDN23, DGAT2, DHCR7, FAXDC2, KRT23, KRT77, SCEL, ACOX2, and ACSL1. In the study of Example 2, all these biomarkers were found to have changed significantly compared to baseline in the qRT-PCR and/or the microarray analysis (Tables 9 and 14).

In embodiments of the invention, the one or more immune effectors are selected from S100A12, S100A9, PI3, CXCL1, MMP12, IL13, CCL17, CCL22, IL8, S100A8, S100A7, IL22, IL17A, IL19, CAMP, DEFB4A/DEFB4B, IL1B, IL6, IL17C, IL15, IL15RA, FOXP3, CXCL10, CCL20, CXCL2, IL12B, IL23A, CCL18, IL10, IL5 and TSLPR, CD86, CCL19, IL24, CCR7, CD2, CD28, CD3D, CD3G, CCL2, CCR1, CCR2, IFNGR2, IL12RB2, IL2RA, IRF1, CCR6, IL6R, LCN2, STAT3, IL37, TNFSF4, S100P, SERPINB1, SERPINB4, CCL13, CCR5, IL4R, IL7R and IL1F10. In the study of Example 2, all these proinflammatory mediators were found to have changed significantly compared to baseline in the qRT-PCR and/or the microarray analysis (Tables 9 and 14).

In embodiments of the invention, the one or more immune effectors are selected from LOR, FLG, KRT16, ANXA6, SPTLC3, CDSN, CERS3, CLN8, ELOVL3, EREG, FA2H, FAR2, KRT79, PNPLA3, PPL, TJP3, ACER1, ANXA9, CLDN1, CLDN23, DGAT2, DHCR7, FAXDC2, KRT23, KRT77, ACOX2, ACSL1 and SCEL. In the study of Example 2, all these skin barrier molecules were found to have changed significantly compared to baseline in the qRT-PCR and/or the microarray analysis (Tables 9 and 14).

In embodiments of the invention, the one or more immune effectors are selected from LOR, FLG, CDSN, CERS3, CLN8, ELOVL3, EREG, FA2H, FAR2, KRT79, PNPLA3, PPL, TJP3, ACER1, ANXA9, CLDN1, CLDN23, DGAT2, DHCR7, FAXDC2, KRT23, KRT77, ACOX2, ACSL1 and SCEL. In the study of Example 2, all these skin barrier molecules were found to have increased significantly compared to baseline in the qRT-PCR and/or the microarray analysis (Tables 9 and 14).

In embodiments of the invention, the one or more immune effectors are selected from S100A12, S100A9, PI3, CXCL1, S100A7, IL17C, CCL20, CCL18, IL10, SPTLC3, CCL2, CCR1, IFNGR2, CCR6, LCN2, STAT3, TNFSF4, CCL13, IL4R, IL1F10, ELOVL3, FA2H, FAR2, KRT79, PNPLA3, DGAT2, FAXDC2 and ACOX2. In the study of Example 2, all these biomarkers were found to have changed significantly compared to baseline and vehicle in the qRT-PCR and/or the microarray analysis (Tables 11 and 16).

In embodiments of the invention, the one or more immune effectors are selected from S100A12, S100A9, PI3, CXCL1, S100A7, IL17C, CCL20, CCL18, IL10, SPTLC3, CCL2, CCR1, IFNGR2, CCR6, LCN2, STAT3, TNFSF4, CCL13, IL4R and IL1F10. In the study of Example 2, all these proinflammatory mediators were found to have changed significantly compared to baseline and vehicle in the qRT-PCR and/or the microarray analysis (Tables 11 and 16).

In embodiments of the invention, the one or more immune effectors are selected from ELOVL3, FA2H, FAR2, KRT79, PNPLA3, DGAT2, FAXDC2 and ACOX2. Particularly, topical application of a halogenated salicylanilide selected from niclosamide and oxycloxanide, or a pharmaceutically acceptable salt or hydrate thereof, may promote expression in lesional skin of one or more immune effectors selected from ELOVL3, FA2H, FAR2, KRT79, PNPLA3, DGAT2, FAXDC2 and ACOX2, which all are skin barrier proteins or skin barrier lipids. In the study of Example 2, all these skin barrier molecules were found to have changed (increased) significantly compared to baseline and vehicle in the qRT-PCR and/or the microarray analysis (Tables 11 and 16).

In embodiments of the invention, the one or more immune effectors are selected from S100A12, S100A9, S100A7, PI3 and CXCL1. In the study of Example 2, all these biomarkers were found to have changed significantly compared to baseline and vehicle in the qRT-PCR and/or the microarray analysis and were all found to correlate with TSS (FIG. 1).

In embodiments of the invention, the one or more immune effectors are selected from S100A12, S100A9, S100A7, PI3 CXCL1, LOR, FLG, CDSN, CERS3, CLN8, ELOVL3, EREG, FA2H, FAR2, KRT79, PNPLA3, PPL, TJP3, ACER1, ANXA9, CLDN1, CLDN23, DGAT2, DHCR7, FAXDC2, KRT23, KRT77, ACOX2, ACSL1 and SCEL.

In embodiments of the invention, the one or more immune effectors are selected from S100A12, S100A9, S100A7, PI3, CXCL1, ELOVL3, FA2H, FAR2, KRT79, PNPLA3, DGAT2, FAXDC2 and ACOX2.

In embodiments of the invention, the one or more immune effectors are selected from CD11c, FceR1 Epidermis, and CD207 cells. In the study of Example 2, all these cell markers were found to have changed significantly compared to baseline in the IHC analysis.

In embodiments of the invention, the one or more immune effectors are CD11c Dermis cells. In the study of Example 2, this cell marker was found to have changed significantly compared to baseline in the IHC analysis and was found to be clinically correlated to TSS (see FIG. 28).

In embodiments of the invention, topical application of a halogenated salicylanilide selected from niclosamide and oxyclozanide (particularly niclosamide), or a pharmaceutically acceptable salt or hydrate thereof, reduces or eliminates one or more clinical signs or symptoms selected from erythema, edema, papulation, lichenification and dryness, particularly one or more clinical signs or symptoms selected from erythema, edema, papulation and lichenification.

In embodiments of the invention, the use of a halogenated salicylanilide selected from niclosamide and oxyclozanide (particularly niclosamide), or a pharmaceutically acceptable salt or hydrate thereof, in the treatment of dermatitis (e.g. atopic dermatitis) in a subject may reduce or eliminate one or more of erythema, edema, papulation, excoriation, lichenification and dryness (xerosis), particularly one or more clinical signs or symptoms selected from erythema, edema, papulation and lichenification.

In embodiments of the invention, the use of a halogenated salicylanilide selected from niclosamide and oxyclozanide (particularly niclosamide), or a pharmaceutically acceptable salt or hydrate thereof, in the treatment of dermatitis (e.g. atopic dermatitis) in a subject, such as a human subject, may reduce or eliminate one or more clinical signs or symptoms selected from of erythema, edema, papulation, lichenification and dryness, and one or more clinical signs or symptoms selected from pruritus, induration, excoriation, scaling, oozing, crusting, lesion nodules, prurigo nodules, lesion vesicles, lesion papules, lesion plaques and lesion swelling associated with the dermatitis (e.g. AD).

In embodiments of the invention, the use of a halogenated salicylanilide selected from niclosamide and oxyclozanide (particularly niclosamide), or a pharmaceutically acceptable salt or hydrate thereof, in the treatment of dermatitis (e.g. atopic dermatitis) in a subject, such as a human subject, may reduce or eliminate one or more clinical signs or symptoms selected from of erythema, edema, papulation, lichenification and dryness, and one or more clinical signs or symptoms selected from pruritus, induration, excoriation, lesion nodules, prurigo nodules, lesion vesicles, lesion papules, lesion plaques and lesion swelling associated with the dermatitis (e.g. AD).

In embodiments of the invention, topical application of a halogenated salicylanilide selected from niclosamide and oxyclozanide (particularly niclosamide), or a pharmaceutically acceptable salt or hydrate thereof, modulates (such as suppresses) expression in lesional skin of one or more immune effectors selected from S100A7, S100A9, PI3, KRT16, IL13, S100A8, DEFB4A/DEFB4B, CCL17, S100A12, IL22, and MMP12, and the topical application reduces or eliminates erythema associated with the inflammatory skin condition (e.g. atopic dermatitis).

In embodiments of the invention, topical application of a halogenated salicylanilide selected from niclosamide and oxyclozanide (particularly niclosamide), or a pharmaceutically acceptable salt or hydrate thereof, suppresses expression in lesional skin of one or more immune effectors selected from S100A7, S100A9, PI3 and S100A12, and the topical application reduces or eliminates erythema associated with the inflammatory skin condition (e.g. atopic dermatitis).

In embodiments of the invention, topical application of a halogenated salicylanilide selected from niclosamide and oxyclozanide (particularly niclosamide), or a pharmaceutically acceptable salt or hydrate thereof, modulates (such as suppresses) expression in lesional skin of IL8, and the topical application reduces or eliminates excoriation associated with the inflammatory skin condition (e.g. atopic dermatitis).

In embodiments of the invention, topical application of a halogenated salicylanilide selected from niclosamide and oxyclozanide (particularly niclosamide), or a pharmaceutically acceptable salt or hydrate thereof, modulates (such as suppresses) expression in lesional skin of one or more immune effectors selected from IL22, S100A9, S100A8, S100A12, S100A7, DEFB4A/DEFB4B, and LOR, and the topical application reduces or eliminates lichenification associated with the inflammatory skin condition (e.g. atopic dermatitis).

In embodiments of the invention, topical application of a halogenated salicylanilide selected from niclosamide and oxyclozanide (particularly niclosamide), or a pharmaceutically acceptable salt or hydrate thereof, suppresses expression in lesional skin of one or more immune effectors selected from S100A9, S100A12 and S100A7, and the topical application reduces or eliminates lichenification associated with the inflammatory skin condition (e.g. atopic dermatitis). In addition, the topical application of the halogenated salicylanilide may promote expression in lesional skin of LOR.

In embodiments of the invention, topical application of a halogenated salicylanilide selected from niclosamide and oxyclozanide (particularly niclosamide), or a pharmaceutically acceptable salt or hydrate thereof, modulates (such as suppresses) expression in lesional skin of one or more immune effectors selected S100A12, S100A9, PI3, KRT16, MMP12, IL13, CCL17, CCL22, S100A8, S100A7, IL22, IL19, DEFB4A/DEFB4B, and LOR, and the topical application reduces or eliminates edema and/or papulation associated with the inflammatory skin condition (e.g. atopic dermatitis).

In embodiments of the invention, topical application of a halogenated salicylanilide selected from niclosamide and oxyclozanide (particularly niclosamide), or a pharmaceutically acceptable salt or hydrate thereof, suppresses expression in lesional skin of one or more immune effectors selected S100A12, S100A9 and PI3, and the topical application reduces or eliminates edema and/or papulation associated with the inflammatory skin condition (e.g. atopic dermatitis). In addition, the topical application of the halogenated salicylanilide may promote expression in lesional skin of LOR.

In embodiments of the invention, topical application of a halogenated salicylanilide selected from niclosamide and oxyclozanide (particularly niclosamide), or a pharmaceutically acceptable salt or hydrate thereof, modulates (such as suppresses) expression in lesional skin of one or more immune effectors is IL13, and the topical application reduces or eliminates dryness associated with the inflammatory skin condition (e.g. atopic dermatitis).

In a further aspect of the invention, there is provided a halogenated salicylanilide selected from niclosamide and oxyclozanide (particularly niclosamide), or a pharmaceutically acceptable salt or hydrate thereof, for use in topical anti-inflammatory treatment of erythema associated with an inflammatory skin condition in a subject (such as a human subject diagnosed with atopic dermatitis) having abnormal (such as elevated) level of one or more immune effectors selected from S100A7, S100A9, PI3, KRT16, IL13, S100A8, DEFB4A/DEFB4B, CCL17, S100A12, IL22, and MMP12, particularly where the subject has abnormal (such as elevated) level of one or more immune effectors selected from S100A7, S100A9, PI3 and S100A12.

In embodiments, the topical anti-inflammatory treatment suppresses expression in lesional skin of one or more immune effectors selected from S100A7, S100A9, PI3, KRT16, IL13, S100A8, DEFB4A/DEFB4B, CCL17, S100A12, IL22, and MMP12, particularly the topical anti-inflammatory treatment suppresses expression in lesional skin of one or more immune effectors selected from S100A7, S100A9, PI3, and S100A12.

In a further aspect of the invention, there is provided a halogenated salicylanilide selected from niclosamide and oxyclozanide (particularly niclosamide), or a pharmaceutically acceptable salt or hydrate thereof, for use in topical anti-inflammatory treatment of excoriation associated with an inflammatory skin condition in a subject (such as a human subject diagnosed with atopic dermatitis) having abnormal (such as elevated) level of IL8.

In embodiments, the topical anti-inflammatory treatment suppresses expression in lesional skin of IL8.

In a further aspect of the invention, there is provided a halogenated salicylanilide selected from niclosamide and oxyclozanide (particularly niclosamide), or a pharmaceutically acceptable salt or hydrate thereof, for use in topical anti-inflammatory treatment of lichenification associated with an inflammatory skin condition in a subject (such as a human subject diagnosed with atopic dermatitis), having abnormal (such as elevated) level of one or more immune effectors selected from IL22, S100A9, S100A8, S100A12, S100A7, and DEFB4A/DEFB4B, particularly where the subject has abnormal (such as elevated) level of one or more immune effectors selected from S100A9, S100A12 and S100A7. The subject may have a deficiency in LOR and the topical anti-inflammatory treatment may promote expression in lesional skin of LOR.

In embodiments, the topical anti-inflammatory treatment suppresses expression in lesional skin of one or more immune effectors selected from IL22, S100A9, S100A8, S100A12, S100A7, and DEFB4A/DEFB4B, particularly the topical anti-inflammatory treatment suppresses expression in lesional skin of one or more immune effectors selected from S100A9, S100A8 and S100A7.

In a further aspect of the invention, there is provided a halogenated salicylanilide selected from niclosamide and oxyclozanide (particularly niclosamide), or a pharmaceutically acceptable salt or hydrate thereof, for use in topical anti-inflammatory treatment of edema and/or papulation associated with an inflammatory skin condition in a subject (such as a human subject diagnosed with atopic dermatitis) having abnormal (such as elevated) level of one or more immune effectors selected from S100A12, S100A9, PI3, KRT16, MMP12, IL13, CCL17, CCL22, S100A8, S100A7, IL22, IL19, DEFB4A/DEFB4B and LOR, particularly the subject has abnormal (such as elevated) level of one or more immune effectors selected from S100A12, S100A9 and PI3. The subject may have a deficiency in LOR and the topical anti-inflammatory treatment may promote expression in lesional skin of LOR.

In embodiments, the topical anti-inflammatory treatment suppresses expression in lesional skin of one or more immune effectors selected S100A12, S100A9, PI3, KRT16, MMP12, IL13, CCL17, CCL22, S100A8, S100A7, IL22, IL19 and DEFB4A/DEFB4B, particularly the topical anti-inflammatory treatment suppresses expression in lesional skin of one or more immune effectors selected from S100A12, S100A9 and PI3.

In a further aspect of the invention, there is provided a halogenated salicylanilide selected from niclosamide and oxyclozanide (particularly niclosamide), or a pharmaceutically acceptable salt or hydrate thereof, for use in topical anti-inflammatory treatment of dryness associated with an inflammatory skin condition in a subject (such as a human subject diagnosed with atopic dermatitis) having abnormal (such elevated) level of IL13 in lesional skin.

In embodiments, the topical anti-inflammatory treatment suppresses expression in lesional skin of IL13.

In embodiments of the invention, the inflammatory skin condition is a clinically uninfected inflammatory skin condition, such as a clinically uninfected atopic dermatitis.

The inflammatory skin condition may be selected from psoriasis, dermatitis, scleroderma, disorders of hair follicles and sebaceous glands, acne, rosacea, rhinophyma, cutaneous lupus, inflammatory reactions (for example drug eruptions, erythema multiforme, erythema nodosum, and granuloma annulare), inflammation associated with fungal or yeast infections (e.g. dermatophytosis), urticaria, dermatitis herpetiformis, lichen planus, hidradenitis suppurativa, pitayriasis rosea, chronic sinusitis, chronic rhinosinusitis, lupus, vitiligo and keratosis pilaris.

In embodiments of the invention, the inflammatory skin condition is not inflammatory acne.

In embodiments of the invention, the inflammatory skin condition is not acne.

The inflammatory skin condition may be selected from psoriasis, dermatitis, scleroderma, disorders of hair follicles and sebaceous glands, rosacea, rhinophyma, cutaneous lupus, inflammatory reactions (for example drug eruptions, erythema multiforme, erythema nodosum, and granuloma annulare), inflammation associated with fungal or yeast infections (e.g. dermatophytosis), urticaria, dermatitis herpetiformis, lichen planus, hidradenitis suppurativa, pitayriasis rosea, chronic sinusitis, chronic rhinosinusitis, lupus, vitiligo and keratosis pilaris.

The inflammatory skin condition may be selected from psoriasis, rosacea and dermatitis (e.g. atopic dermatitis).

In a further aspect of the invention, there is provided a halogenated salicylanilide selected from niclosamide and oxyclozanide (particularly niclosamide), or a pharmaceutically acceptable salt or hydrate thereof, for the use in topical anti-inflammatory treatment of one or more clinical signs or symptoms of dermatitis (e.g. atopic dermatitis) in a subject, wherein the one or more clinical signs or symptoms are selected from erythema, excoriation, lichenification, edema, papulation and dryness, particularly erythema, lichenification, edema and papulation. The the one or more clinical signs or symptoms selected from erythema, excoriation, lichenification, edema, papulation and dryness may be associated with an abnormal inflammatory response.

In embodiments of the invention, the inflammatory skin condition is dermatitis, for example atopic dermatitis.

The dermatitis may be, for example a dermatitis (or eczema) selected from contact dermatitis, allergic contact dermatitis, irritant contact dermatitis, atopic dermatitis, seborrhoeic dermatitis, actinic dermatitis, hand and foot dermatitis, pompholyx dermatitis, lichen simplex chronicus (neurodermatitis), exfoliative dermatitis (erythroderma), asteatotic dermatitis, carcinomatous dermatitis, nummular dermatitis, neonatal dermatitis, paediatric dermatitis, diaper dermatitis, stasis dermatitis, perioral dermatitis, dermatomyositis, eczematous dermatitis, photoallergic dermatitis, phototoxic dermatitis, phytophotodermatitis and radiation-induced dermatitis.

In embodiments of the invention, the use of a halogenated salicylanilide selected from niclosamide and oxyclozanide (particularly niclosamide), or a pharmaceutically acceptable salt or hydrate thereof, in the treatment of dermatitis (e.g. atopic dermatitis) in a subject to may reduce or eliminate one or more of pruritus, erythema, induration, excoriation, lichenification, scaling, oozing, crusting, xerosis, lesion nodules, prurigo nodules, lesion vesicles, lesion papules, lesion plaques, lesion swelling, hypopigmentation or hyperpigmentation associated with the dermatitis (e.g. atopic dermatitis).

Topical application of a halogenated salicylanilide selected from niclosamide and oxyclozanide (particularly niclosamide), or a pharmaceutically acceptable salt or hydrate thereof, may be used for treatment of an exacerbation of dermatitis.

The dermatitis may be mild, moderate or severe dermatitis.

The dermatitis may be an acute form of dermatitis or a chronic form of dermitits.

In embodiments of the invention, the inflammatory skin condition is atopic dermatitis.

In a further aspect of the invention, there is provided a halogenated salicylanilide selected from niclosamide and oxyclozanide (particularly niclosamide), or a pharmaceutically acceptable salt or hydrate thereof, for the use in topical anti-inflammatory treatment of one or more clinical signs or symptoms of rosacea in a subject, wherein the one or more clinical signs or symptoms are selected from erythema, excoriation, lichenification, edema, papulation and dryness, particularly erythema, lichenification, edema and papulation. The the one or more clinical signs or symptoms selected from erythema, excoriation, lichenification, edema, papulation and dryness may be associated with an abnormal inflammatory response.

In a further aspect of the invention, there is provided a halogenated salicylanilide selected from niclosamide and oxyclozanide (particularly niclosamide), or a pharmaceutically acceptable salt or hydrate thereof, for the use in topical anti-inflammatory treatment of one or more clinical signs or symptoms of psoriasis in a subject, wherein the one or more clinical signs or symptoms are selected from erythema, excoriation, lichenification, edema, papulation and dryness, particularly erythema, lichenification, edema and papulation. The the one or more clinical signs or symptoms selected from erythema, excoriation, lichenification, edema, papulation and dryness may be associated with an abnormal inflammatory response.

In embodiments of the invention, the one or more clinical signs or symptoms are associated with a Th1, Th2, Th17 and/or Th22-type inflammatory response.

In embodiments of the invention, a halogenated salicylanilide selected from niclosamide and oxyclozanide (particularly niclosamide), or a pharmaceutically acceptable salt or hydrate thereof, is topically applied to inflammatory lesional skin of the subject to be treated.

In embodiments of the invention, the inflammatory skin condition is atopic dermatitis and topical application of a halogenated salicylanilide selected from niclosamide and oxyclozanide (particularly niclosamide), or a pharmaceutically acceptable salt or hydrate thereof, and topical application of a halogenated salicylanilide selected from niclosamide and oxyclozanide (particularly niclosamide), or a pharmaceutically acceptable salt or hydrate thereof, reduces the Total Sign Score (TSS) of atopic dermatitis by more than 10%, such as at least 20%.

In a still further aspect of the invention, there is provided a halogenated salicylanilide selected from niclosamide and oxyclozanide (particularly niclosamide), or a pharmaceutically acceptable salt or hydrate thereof, for use in topical anti-inflammatory treatment of one or more clinical signs or symptoms of an inflammatory ocular condition, such as dry eye disorder (DED), in a subject, wherein the one or more clinical signs or symptoms are associated with an abnormal inflammatory response, and the topical anti-inflammatory treatment modulates (such as decreases/downregulates) expression of one or more immune effectors selected from proinflammatory mediators and ocular surface epithelial barrier molecules. The inflammatory ocular condition may be associated with a dysfunction of pre-corneal tear film and/or ocular surface epithelial barrier. The topical anti-inflammatory treatment may modulate (such as decreases/downregulates) expression of one or more immune effectors selected from proinflammatory mediators and ocular surface epithelial barrier molecules in ocular and ocular-associated tissue, such as adnexa, conjunctiva and cornea, and/or in pre-corneal tear film. The topical anti-inflammatory treatment may increase tear production in patients experiencing a dysfunction of pre-corneal tear film associated with an inflammatory ocular disease.

In embodiments, the inflammatory ocular condition is elected from dry eye disorder (DED), ocular roacea, uveitis (e.g. Birdshot retinochoroidopathy), severe conjunctivitis, diabetic retinopathy, multifocal choroiditis with panuveitis, serpiginous choroidopathy, scleritis, an eye inflammation associated with allergy, and an eye inflammation associated with an autoimmune disorder (e.g. mucos membrane pempigoid, ankylosing spondylitis, Behcet's syndrome, dermatomyositis, Graves' disease, juvenile rheumatoid arthritis, multiple sclerosis, psoriatic arthritis, Reiter's syndrome, rheumatoid arthritis, Sjogren's syndrome, systemic lupus erythematosus, and Wegener's granulatomatosis).

In embodiments, the topical anti-inflammatory treatment provides decreased expression in ocular and ocular-associated tissue (e.g. cornea) and/or in pre-corneal tear film of one or more proinflammatory mediators.

In embodiments, the topical anti-inflammatory treatment provides increased expression in ocular and ocular-associated tissue (e.g. cornea) of one or more ocular surface epithelial barrier molecules.

In embodiments, the one or more clinical signs or symptoms are associated with an abnormal (such as elevated) level of one or more proinflammatory mediators and the topical anti-inflammatory treatment reduces the abnormal level of said one or more proinflammatory mediators in ocular and ocular-associated tissue (e.g. cornea) and/or in pre-corneal tear film.

In embodiments, the one or more clinical signs or symptoms are associated with deficiency in one or more ocular surface epithelial barrier molecules and the topical anti-inflammatory treatment provides increased expression in ocular and ocular-associated tissue (e.g. cornea) of one or more ocular surface epithelial barrier molecules.

In embodiments, the inflammatory ocular condition is dry eye disease (DED).

In embodiments, the one or more immune effectors are selected from proinflammatory mediators.

In embodiments, the proinflammatory mediators are selected from proinflammatory cytokines, proinflammatory enzymes, antibacterial proteins and peptides, and immune cells.

In embodiments, the proinflammatory mediators selected from proinflammatory cytokines, proinflammatory enzymes and immune cells.

In embodiments, the ocular surface epithelial barrier molecules are selected from structural ocular surface epithelial barrier proteins (e.g. LOR and FLG) and ocular surface epithelial barrier lipids. Structural ocular surface epithelial barrier proteins, such as LOR and FLG, are expressed by corneal epithelial cells (Tong et al, Invest Ophthalmol Vis Sci, 47(5): 1938-1946, 2006).

In embodiments, the abnormal inflammatory response involves a Th1, Th2, Th17 and/or Th22-type inflammatory response.

In embodiments, the expression in ocular and ocular-associated tissue (e.g. cornea) and/or pre-corneal tear film of said one or more immune effectors are associated with activation of Th1, Th2, Th17 and/or Th22 cells.

In embodiments, expression of said one or more immune effectors is modulated by attenuating one or more responses selected from Th1, Th2, Th17 and Th22-type inflammatory response.

In embodiments, the one or more immune effectors are selected from S100A12, S100A9, PI3, CXCL1, KRT16, MMP12, IL13, CCL17, CCL22, IL8, S100A8, S100A7, IL22, IL17A, IL19, CAMP, DEFB4A/DEFB4B, LOR, IL1B, IL6, IL17C, IL15, IL15RA, FOXP3, FLG, CXCL10, CCL20, CXCL2, IL12B, IL23A, CCL18, IL10, IL5, TSLPR, CD86, CCL19, IL24, ANXA6, SPTLC3, CCR7, CD2, CD28, CD3D, CD3G, CCL2, CCR1, CCR2, IFNGR2, IL12RB2, IL2RA, IRF1, CCR6, IL6R, LCN2, STAT3, IL37, TNFSF4, S100P, SERPINB1, SERPINB4, CCL13, CCR5, IL4R, IL7R, IL1F10, CDSN, CERS3, CLN8, ELOVL3, EREG, FA2H, FAR2, KRT79, PNPLA3, PPL, TJP3, ACER1, ANXA9, CLDN1, CLDN23, DGAT2, DHCR7, FAXDC2, KRT23, KRT77, SCEL, ACOX2 and ACSL1.

In embodiments, the one or more immune effectors are selected from S100A12, S100A9, PI3, CXCL1, S100A7, IL17C, CCL20, CCL18, IL10, SPTLC3, CCL2, CCR1, IFNGR2, CCR6, LCN2, STAT3, TNFSF4, CCL13, IL4R, IL1F10, ELOVL3, FA2H, FAR2, KRT79, PNPLA3, DGAT2, FAXDC2 and ACOX2.

In embodiments, the one or more immune effectors are selected from S100A12, S100A9, PI3, CXCL1, S100A7, IL17C, CCL20, CCL18, IL10, SPTLC3, CCL2, CCR1, IFNGR2, CCR6, LCN2, STAT3, TNFSF4, CCL13, IL4R and IL1F10.

In embodiments, the one or more immune effectors are selected from ELOVL3, FA2H, FAR2, KRT79, PNPLA3, DGAT2, FAXDC2 and ACOX2.

In embodiments, the one or more immune effectors are selected from LOR, FLG, KRT16, ANXA6, SPTLC3, CDSN, CERS3, CLN8, ELOVL3, EREG, FA2H, FAR2, KRT79, PNPLA3, PPL, TJP3, ACER1, ANXA9, CLDN1, CLDN23, DGAT2, DHCR7, FAXDC2, KRT23, KRT77 and SCEL.

In embodiments of the invention, the one or more immune effectors are selected from LOR, FLG, ELOVL3, FA2H, FAR2, KRT79, PNPLA3, DGAT2, FAXDC2 and ACOX2.

In embodiments of the invention, the one or more immune effectors are selected from ELOVL3, FA2H, FAR2, KRT79, PNPLA3, DGAT2, FAXDC2 and ACOX2.

In embodiments, the one or more immune effectors are selected from S100A12, S100A9, PI3, CXCL1, KRT16, MMP12, IL13, CCL17, CCL22, IL8, S100A8, S100A7, IL22, IL17A, IL19, CAMP, DEFB4A/DEFB4B, LOR, IL1B, IL6, IL17C, IL15, IL15RA, FOXP3, FLG, CXCL10, CCL20, CXCL2, IL12B, IL23A, CCL18, IL10, IL5 and TSLPR.

In embodiments, the one or more immune effectors are selected from S100A12, S100A9, S100A7, PI3 and CXCL1.

In embodiments of the invention, the one or more immune effectors are selected from S100A12, S100A9, S100A7, PI3 CXCL1, LOR, FLG, ELOVL3, FA2H, FAR2, KRT79, PNPLA3, DGAT2, FAXDC2 and ACOX2.

In embodiments, the one or more immune effectors are selected from S100A12, S100A9, S100A7, PI3 CXCL1, ELOVL3, FA2H, FAR2, KRT79, PNPLA3, DGAT2, FAXDC2 and ACOX2.

For example, S100A8 and S100A9 are considered biomarkers for dry eye disease (DED) (Enriquez-de-Salamanaca et al, “Molecular and cellular biomarkers in dry eye disease and ocular allergy”, Current Opinion in Allergy and Clinical Immunology, 12(5):523-533, 2012).

In embodiments, a halogenated salicylanilide selected from niclosamide and oxyclozanide (particularly niclosamide), or a pharmaceutically acceptable salt or hydrate thereof, is topically administered in the form an ophthalmic composition, such as an ophthalmic cream, ointment, gel, paste, lotion, foam, suspension and solution.

In a further aspect of the invention, there is provided a halogenated salicylanilide selected from niclosamide and oxyclozanide (particularly niclosamide), or a pharmaceutically acceptable salt or hydrate thereof, for the use in topical anti-inflammatory treatment of one or more clinical signs or symptoms of dry eye disease (DED) in a subject. Particularly, the one or more clinical signs or symptoms are associated with an abnormal inflammatory response.

A further aspect of the invention provides the use of a halogenated salicylanilide selected from niclosamide and oxyclozanide (particularly niclosamide), or a pharmaceutically acceptable salt or hydrate thereof, in the manufacture of a medicament for topical anti-inflammatory treatment of one or more clinical signs or symptom, such as erythema, excoriation, lichenification, edema, papulation and dryness, of an inflammatory skin condition in a subject. The use is applicable to all aspects and embodiments of the treatment of inflammatory skin condition using nicloasamide, or a pharmaceutically acceptable salt or hydrate thereof, as described herein.

A further aspect of the invention provides the use of a halogenated salicylanilide selected from niclosamide and oxyclozanide (particularly niclosamide), or a pharmaceutically acceptable salt or hydrate thereof, in the manufacture of a medicament for topical anti-inflammatory treatment of one or more clinical signs or symptom of an inflammatory ocular condition in a subject. The use is applicable to all aspects and embodiments of the treatment of inflammatory ocular conditions using nicloasamide, or a pharmaceutically acceptable salt or hydrate thereof, as described herein.

A further aspect of the invention provides a method for treating (such as reducing or eliminating) one or more clinical signs or symptoms of an inflammatory skin condition in a subject, wherein the one or more clinical signs or symptoms are associated with an abnormal inflammatory response, the method comprising topically administrating (applying) a therapeutically effective amount of a halogenated salicylanilide selected from niclosamide and oxyclozanide (particularly niclosamide), or a pharmaceutically acceptable salt or hydrate thereof, to the skin (including lesional skin) of the subject. The method is applicable to all aspects and embodiments of the treatment of inflammatory skin conditions using nicloasamide, or a pharmaceutically acceptable salt or hydrate thereof, as described herein.

In embodiments, the one or more clinical signs or symptoms are associated with skin barrier dysfunction.

In embodiments, decreased expression in lesional skin of one or more proinflammatory mediators is provided.

In embodiments, increased expression in lesional skin of one or more skin barrier molecules is provided.

In embodiments, the one or more clinical signs or symptoms are selected from erythema, excoriation, lichenification, edema, papulation, dryness, pruritus, scaling, oozing and crusting, such as erythema, excoriation, lichenification, edema, papulation and dryness, particularly erythema, lichenification, edema and papulation.

In embodiments, the one or more clinical signs or symptoms are associated with an abnormal (such as elevated) level of one or more proinflammatory mediators in lesional skin and the topical anti-inflammatory treatment reduces the abnormal level of said one or more proinflammatory mediators in lesional skin.

In embodiments, the one or more clinical signs or symptoms are associated with skin barrier dysfunction including a deficiency in one or more skin barrier molecules and the topical anti-inflammatory treatment increases expression in lesional skin of one or more skin barrier molecules.

A further aspect of the invention provides a method for modulating (such as attenuating) an abnormal inflammatory response associated with an inflammatory skin condition in a subject, the method comprising topically administrating (applying) a therapeutically effective amount of a halogenated salicylanilide selected from niclosamide and oxyclozanide (particularly niclosamide), or a pharmaceutically acceptable salt or hydrate thereof, to the skin (including lesional skin) of the subject, and thereby providing modulated expression in lesional skin of immune effectors selected from proinflammatory mediators and skin barrier molecules, including providing decreased expression in lesional skin of one or more proinflammatory mediators, and increased expression in lesional skin of one or more skin barrier molecules. The method is applicable to all aspects and embodiments for modulating an abnormal inflammatory response associated with an inflammatory skin condition using nicloasamide, or a pharmaceutically acceptable salt or hydrate thereof, as described herein.

A further aspect of the invention provides a method for improving skin barrier function associated with an inflammatory skin condition in a subject, the method comprising topically administrating (applying) a therapeutically effective amount of a halogenated salicylanilide selected from niclosamide and oxyclozanide (particularly niclosamide), or a pharmaceutically acceptable salt or hydrate thereof, to the skin (including lesional skin) of the subject, and thereby providing modulated expression in lesional skin of one or more immune effectors selected from proinflammatory mediators and skin barrier molecules, such as providing decreased expression in lesional skin of one or more proinflammatory mediators, and increased expression in lesional skin of one or more skin barrier molecules. The method is applicable to all aspects and embodiments for improving skin barrier function associated with an inflammatory skin condition using nicloasamide, or a pharmaceutically acceptable salt or hydrate thereof, as described herein.

A further aspect of the invention provides a method for treating erythema associated with an inflammatory skin condition in a subject having abnormal (such as elevated) level of one or more immune effectors selected from S100A7, S100A9, PI3, KRT16, IL13, S100A8, DEFB4A/DEFB4B, CCL17, S100A12, IL22, and MMP12 in lesional skin, particularly abnormal (such as elevated) level of one or more immune effectors selected from S100A7, S100A9, PI3 and S100A12, in lesional skin, the method comprising topically administrating (applying) a therapeutically effective amount of a halogenated salicylanilide selected from niclosamide and oxyclozanide (particularly niclosamide), or a pharmaceutically acceptable salt or hydrate thereof, to the skin (including lesional skin) of the subject. The method is applicable to all aspects and embodiments for treating erythema associated with an inflammatory skin condition using nicloasamide, or a pharmaceutically acceptable salt or hydrate thereof, as described herein.

A further aspect of the invention provides a method for treating excoriation associated with an inflammatory skin condition in a subject having abnormal (such as elevated) level of IL8 in lesional skin, the method comprising topically administrating (applying) a therapeutically effective amount of a halogenated salicylanilide selected from niclosamide and oxyclozanide (particularly niclosamide), or a pharmaceutically acceptable salt or hydrate thereof, to the skin (including lesional skin) of the subject. The method is applicable to all aspects and embodiments for treating excoriation associated with an inflammatory skin condition using nicloasamide, or a pharmaceutically acceptable salt or hydrate thereof, as described herein.

A further aspect of the invention provides a method for treating lichenification associated with an inflammatory skin condition in a subject having abnormal (such as elevated) level of one or more immune effectors selected from IL22, S100A9, S100A8, S100A12, S100A7, and DEFB4A/DEFB4B in lesional skin, particularly abnormal (such as elevated) level of one or more immune effectors selected from S100A9, SA10012 and S100A7 in lesional skin, the method comprising topically administrating (applying) a therapeutically effective amount of a halogenated salicylanilide selected from niclosamide and oxyclozanide (particularly niclosamide), or a pharmaceutically acceptable salt or hydrate thereof, to the skin (including lesional skin) of the subject. The method is applicable to all aspects and embodiments for treating lichenification associated with an inflammatory skin condition using nicloasamide, or a pharmaceutically acceptable salt or hydrate thereof, as described herein.

A further aspect of the invention provides a method for treating edema and/or papulation associated with an inflammatory skin condition in a subject having abnormal (such as elevated) level of one or more immune effectors selected from S100A12, S100A9, PI3, KRT16, MMP12, IL13, CCL17, CCL22, S100A8, S100A7, IL22, IL19 and DEFB4A/DEFB4B in lesional skin, particularly abnormal (such as elevated) level of one or more immune effectors selected from S100A12, S100A9 and PI3, in lesional skin, the method comprising topically administrating (applying) a therapeutically effective amount of a halogenated salicylanilide selected from niclosamide and oxyclozanide (particularly niclosamide), or a pharmaceutically acceptable salt or hydrate thereof, to the skin (including lesional skin) of the subject. The method is applicable to all aspects and embodiments for treating edema and/or papulation associated with an inflammatory skin condition using nicloasamide, or a pharmaceutically acceptable salt or hydrate thereof, as described herein.

A further aspect of the invention provides a method for treating dryness associated with an inflammatory skin condition in a subject having abnormal (such as elevated) level of IL13 in lesional skin, the method comprising topically administrating (applying) a therapeutically effective amount of a halogenated salicylanilide selected from niclosamide and oxyclozanide (particularly niclosamide), or a pharmaceutically acceptable salt or hydrate thereof, to the skin (including lesional skin) of the subject. The method is applicable to all aspects and embodiments for treating dryness associated with an inflammatory skin condition using nicloasamide, or a pharmaceutically acceptable salt or hydrate thereof, as described herein.

A further aspect of the invention provides a method of treating one or more clinical signs or symptoms of dermatitis (e.g. atopic dermatitis) in a subject, wherein the one or more clinical signs or symptoms are selected from erythema, excoriation, lichenification, edema, papulation and dryness, particularly one or more clinical signs or symptoms are selected from erythema, lichenification, edema and papulation, the method comprising topically administrating (applying) a therapeutically effective amount of a halogenated salicylanilide selected from niclosamide and oxyclozanide (particularly niclosamide), or a pharmaceutically acceptable salt or hydrate thereof, to the skin (including lesional skin) of the subject.

A further aspect of the invention provides a method of treating one or more clinical signs or symptoms of rosacea in a subject, wherein the one or more clinical signs or symptoms are selected from erythema, excoriation, lichenification, edema, papulation and dryness, particularly one or more clinical signs or symptoms are selected from erythema, lichenification, edema and papulation, the method comprising topically administrating (applying) a therapeutically effective amount of a halogenated salicylanilide selected from niclosamide and oxyclozanide (particularly niclosamide), or a pharmaceutically acceptable salt or hydrate thereof, to the skin (including lesional skin) of the subject.

A further aspect of the invention provides a method of treating one or more clinical signs or symptoms of psoriasis in a subject, wherein the one or more clinical signs or symptoms are selected from erythema, excoriation, lichenification, edema, papulation and dryness, particularly one or more clinical signs or symptoms are selected from erythema, lichenification, edema and papulation, the method comprising topically administrating (applying) a therapeutically effective amount of a halogenated salicylanilide selected from niclosamide and oxyclozanide (particularly niclosamide), or a pharmaceutically acceptable salt or hydrate thereof, to the skin (including lesional skin) of the subject.

A still further aspect of the invention provides a method of treating one or more clinical signs or symptoms of an inflammatory ocular condition in a subject, wherein the one or more clinical signs or symptoms are associated with an abnormal inflammatory response and pre-corneal tear film dysfunction, the method comprising topically administrating (applying) a therapeutically effective amount of a halogenated salicylanilide selected from niclosamide and oxyclozanide (particularly niclosamide), or a pharmaceutically acceptable salt or hydrate thereof, to the eye of the subject, and thereby providing modulated (such as decreased/downregulated) expression of one or more immune effectors, such as modulated expression of one or more immune effectors in ocular and ocular-associated tissue (e.g. cornea) and/or in pre-corneal tear film. The method is applicable to all aspects and embodiments of the treatment of inflammatory ocular conditions using nicloasamide, or a pharmaceutically acceptable salt or hydrate thereof, as described herein.

A still further aspect of the invention provides a method of treating one or more clinical signs or symptoms of dry eye disease in a subject, the method comprising topically administrating (applying) a therapeutically effective amount of a halogenated salicylanilide selected from niclosamide and oxyclozanide (particularly niclosamide), or a pharmaceutically acceptable salt or hydrate thereof, to the eye of the subject.

In embodiments of the invention, the subject is a human.

In embodiments of the invention, the subject is an adult human.

In embodiments of the invention, the subject is a paediatric human, for example a human less than 18 years old.

In embodiments of the invention, the halogenated salicylanilide is niclosamide, or a pharmaceutically acceptable salt or hydrate thereof, particularly the halogenated salicylanilide is niclosamide.

In embodiments of the invention, the halogenated salicylanilide is oxyclozanide, or a pharmaceutically acceptable salt or hydrate thereof.

Further aspects and features of the invention are set out in the detailed description below.

### BRIEF SUMMARY OF DRAWINGS

FIG. 1 shows the changes in biomarker expression that correlated with TSS/TAA and were found to have significantly changed compared to vehicle and baseline (S100A12, S100A9, PI3, CXCL1 and S100A7) as analysed in skin biopsies taken at Day 22 in the study of Example 2.

FIGS. 2-5 show the changes in biomarker expression (KRT16, MMP12, IL13, CCL17, CCL22, IL8, LOR, FLG, CD11c Dermis, S100A8, S100A12, S100A7, S100A9, IL22, PI3, CXCL1, IL17A, IL19, CAMP and DEFB4A/DEFB4B) that were found to correlate with TSS and were found to have significantly changed compared to baseline as analysed in skin biopsies taken at Day 22 in the study of Example 2.

FIG. 6 shows the correlation between individual scores (erythema, edema/papulation, oozing/crusting, excoriation, lichenification and dryness) and TSS as found in the study of Example 2.

FIG. 7 shows the changes in expression of biomarkers (IL13, S100A7, S100A8, KRT16, IL22, S100A9, S100A12, CCL17, MMP12, PI3, CCL22, DEFB4A/DEFB4B, IL19 and LOR) that correlated with edema/papulation and were found to have significantly changed compared to baseline as analysed in skin biopsies taken at Day 22 in the study of Example 2.

FIG. 8 shows the changes in expression of biomarkers (S100A7, S100A9, KRT16, IL13, S100A8, DEFB4A/DEFB4B, PI3, CCL17, S100A12, IL22 and MMP12) that correlated with erythema and were found to have significantly changed compared to baseline as analysed in skin biopsies taken at Day 22 in the study of Example 2.

FIG. 9 shows the changes in expression of biomarkers (IL22, S100A7, S100A8, S100A12, DEFB4A/DEFB4B, S100A9 and LOR) that correlated with lichenification and were found to have significantly changed compared to baseline as analysed by in skin biopsies taken at Day 22 in the study of Example 2.

FIG. 10 shows the changes in expression of biomarkers (IL13) that correlated with dryness and were found to have significantly changed compared to baseline as analysed in skin biopsies taken at Day 22 in the study of Example 2.

FIG. 11 shows the changes in expression of biomarkers (IL8) that correlated with excoriation and were found to have significantly changed compared to baseline as analysed in skin biopsies at Day 22 in the study of Example 2.

FIGS. 12-15 show the changes in biomarker expression (KRT16, MMP12, IL13, CCL17, CCL22, IL8, LOR, FLG, S100A8, S100A12, S100A7, S100A9, IL22, PI3, DEFB4A/DEFB4B, IL19) that were found to correlate with TAA and were found to have significantly changed compared to baseline as analysed in skin biopsies taken at Day 22 in the study of Example 2.

FIGS. 16-25 show changes in biomarker expression (IL6, IL8, IL17C, IL1B, IL15, IL15RA, IL2, CCL5, IFNG, CXCL9, IL12A/IL12p35, CXCL10, IL13, IL10, IL33, TSLP-R, IL31, IL5, CCL17, CCL18, CCL22, CCL26, IL17A, IL17F, IL23A/IL23p19, CAMP/LL37, IL19, IL12B/IL23p40, DEFB4A/DEFB4B, CXCL1, CXCL2, CCL20, PI3, IL22, S100A7, S100A8, S100A9, S100A12, FLG, PPL, LOR, KRT16, MMP12, IL9 and FOXP3) for vehicle (A) and niclosamide (B) compared to baseline as analysed in skin biopsies taken at Day 22 in the study of Example 2. FCH stands for fold change.

FIGS. 26-29 show changes in cell markers (CD3, langerin, CD11c and FceR1) for vehicle (A) and niclosamide (B) compared to baseline as analysed in skin biopsies taken at Day 22 in the study of Example 2.

### DETAILED DESCRIPTION

### Definitions

Unless otherwise stated, the following terms used in the specification and claims have the following meanings set out below.

The terms “treating” or “treatment” refers to any indicia of success in the treatment or amelioration of a disease, pathology or condition, including any objective or subjective parameter such as abatement; remission; diminishing of symptoms or making the pathology or condition more tolerable to the patient; slowing in the rate of degeneration or decline; making the final point of degeneration less debilitating; improving a patient's physical or mental well-being. For example, certain methods herein treat dermatitis (e.g. AD) by decreasing a symptom of dermatitis (e.g. AD). Symptoms of dermatitis are known or may be readily determined by a person of ordinary skill in the art. The term “treating” and conjugations thereof, include prevention of a pathology, condition, or disease (e.g.

preventing the development of one or more symptoms of dermatitis (e.g. AD).

The term “associated” or “associated with” in the context of a substance or substance activity or function associated with a disease (e.g. AD) means that the disease is caused by (in whole or in part), or a symptom of the disease is caused by (in whole or in part) the substance or substance activity or function.

When a compound or salt described in this specification is administered to treat a disorder, a “therapeutically effective amount” is an amount sufficient to reduce or completely alleviate symptoms or other detrimental effects of the disorder; cure the disorder; reverse, completely stop, or slow the progress of the disorder; or reduce the risk of the disorder getting worse.

Colony-forming unit (CFU) is an approximate estimate of the number of viable bacterial cells in a sample. Viable is defined as the ability of the cell to multiply via binary fission under the controlled conditions.

The term “pharmaceutically acceptable salt” refers to salts that retain the biological effectiveness and properties of the compounds described herein and, which are not biologically or otherwise undesirable. Pharmaceutically acceptable salts are well known to skilled persons in the art. Particular salts include ethanolamine or piperazine salts. Accordingly, it may be that a reference to a salt of niclosamide herein may refer to a pharmaceutically acceptable salt of niclosamide.

The term “solvate” is used herein to refer to a complex of solute, such as a compound or salt of the compound, and a solvent. If the solvent is water, the solvate may be termed a hydrate, for example a monohydrate, dihydrate, trihydrate etc., depending on the number of water molecules present per molecule of substrate. Reference to “a halogenated salicylanilide selected from niclosamide and oxyclozanide (particularly niclosamide), or a pharmaceutically acceptable salt or hydrate thereof” includes hydrates of niclosamide or oxyclozanide and hydrates of a salt of niclosamide or oxyclozanide.

Reference to “PEG x00” herein means a polyethylene glycol with an average molecular weight of x00. For example, PEG 400 refers to a PEG with an average molecular weight of 400. Unless stated otherwise reference herein to the molecular weight of polymer, such as a PEG is a reference to number average molecular weight (Mn) of the polymer. The number average molecular weight can be measured using well known methods, for example by gel permeation chromatography or 1H NMR end-group analysis. Such methods include GPC analysis as described in Guadalupe et al (Handbook of

Polymer Synthesis, Characterization, and Processing, First Edition, 2013) and end group analysis described in e.g. Page et al Anal. Chem., 1964, 36 (10), pp 1981-1985.

As used herein “abnormal” level of a proinflammatory mediator is referred to be associated with unregulated expression (including recruitment) and/or retention in for example tissue, such as skin, of said one or more proinflammatory mediators.

As used herein “abnormal inflammatory response” is associated with an abnormal level of one or more proinflammatory mediators in for example tissue, such as lesional skin.

As used herein “modulate/supress/promote/increase/decrease expression” refers to an effect on any mechanism, such as mediator secretion, cell proliferation, gene expression and protein production, which may affect the occurrence of an immune effector, such as a proinflammatory mediator or a skin barrier molecule in for example tissue, such as skin.

As used herein “endogenous expression” refers to expression of an immune effector originating from within the subject to be treated.

As used herein “modulate/supress/promote/increase/decrease” in relation to (endogenous) expression of an immune effector refers to the change in expression of said immune effector in the sample analysed (e.g. in a skin biopsy analysed by qRT-PCT, microarray or immunohistochemistry as disclosed herein) post-administration with a therapeutically effective amount of a halogenated salicylanilide selected from niclosamide and oxyclozanide (particularly niclosamide) (or a pharmaceutically acceptable salt or hydrate thereof) compared to pre-administration (i.e. baseline). A halogenated salicylanilide selected from niclosamide and oxyclozanide (particularly niclosamide), or a pharmaceutically acceptable salt thereof, may be topically applied to inflammatory lesional skin 1, 2, 3 or 4 times per day. The duration of the treatment may be, for example, 1 week or more, 2 weeks or more, 3 weeks or more, such as once daily for 22 days. A halogenated salicylanilide selected from niclosamide and oxyclozanide (particularly niclosamide), or a pharmaceutically acceptable salt or hydrate thereof, may, for example, be topically administered 1, 2 or 3 times per week for 1 week or more, 2 weeks or more, 3 weeks or more.

As used herein “attenuating an inflammatory response” refers to a diminishing effect on the inflammatory response post-administration with a halogenated salicylanilide selected from niclosamide and oxyclozanide (particularly niclosamide) (or a pharmaceutically acceptable salt or hydrate thereof) compared to pre-administration (i.e. baseline). The treatment period may be at least once daily for more than 5 days, such as 22 days. The diminishing effect of the inflammatory response can be measured by analysing the change in expression of one or more proinflammatory mediators in the sample analysed (e.g. in a skin biopsy analysed by qRT-PCT, microarray or immunohistochemistry as disclosed herein) post-administration with a halogenated salicylanilide selected from niclosamide and oxyclozanide (particularly niclosamide) (or a pharmaceutically acceptable salt or hydrate thereof) compared to pre-administration (i.e. baseline).

As used herein the term “proinflammatory” refers to an inflammation-promoting effect.

As used herein the term “proinflammatory mediator” refers to molecules and cells having an inflammation-promoting effect and being functionally involved in the signalling pathways and the immune responses of the immune system.

As used herein “immune effectors” refers to cells and molecules having a function in the inflammatory response of the immune system, such as immune cells, proinflammatory cytokines, proinflammatory enzymes and skin barrier molecules.

As used herein “innate immune effectors” refers to cells and molecules having a function in the inflammatory response of the innate immune system, such as innate immune cells, innate proinflammatory cytokines, innate proinflammatory enzymes and innate antibacterial peptides and proteins.

As used herein “immune cells” refers to cells involved in the innate and adaptive immune systems.

Examples of innate immune cells in the skin barrier are Langerhans cells (which e.g. includes langerin, also called CD207), dendritic cells (which e.g. includes CD11c, FceR1), keratinocytes (KCs), natural killer (NK) cells, mast cells, and polymorphonuclear neutrophils (PMNs).

As used herein “skin barrier molecules” refers to skin barrier proteins, skin barrier peptides and skin barrier lipids present in the stratum corneum of the epidermis.

Examples of skin barrier lipids are ACOX2, ELOVL3, FA2H, FAR2, KRT79, and PNPLA3. Examples of skin barrier peptides and proteins are defensins (e.g. defensin beta 4B/4A (DEFB4B/DEFB4A)), S100 proteins (e.g. S100A7, S100A8, S100A9, S100A12), filaggrin (FLG), keratins (e.g. keratin-16 (K16 or KRT16)), loricrin (LOR), cathelicidins (cathelicidin antimicrobial peptide, CAMP), and PI3 (peptidase inhibitor 3, also called elafin) (Scahuber et al, J Allergy Clin Immunol, 12(2):261-266, 2008).

Some skin barrier peptides and proteins are secretory peptides and proteins with antimicrobial activity against pathogenic microbes. These secretory skin barrier peptides and proteins are referred to as antimicrobial peptides and proteins (AMPs). Examples of antimicrobial peptides and proteins (AMPs) are defensins, cathelicidins, S100 proteins and PI3. Most antimicrobial peptides and proteins (e.g. S100A8, S100A9 and S100A12) have a proinflammatory function in addition to their antimicrobial activities and are believed to trigger and coordinate multiple components of the innate and adaptive immune system. These secretory skin barrier peptides and proteins may therefore also be referred to as proinflammatory mediators.

Other skin barrier proteins, such as filaggrin (FLG), periplakin (PPL), keratins and loricrin (LOR), are structural proteins, such as structural proteins of the stratum corneum.

As used herein the terms “cytokine” are signalling molecules of the immune system and refers to small proteins released by cells (e.g. lymphocytes, monocytes, macrophages and leukocytes) that mediate communication and interaction between cells. Examples of cytokines are interleukins (e.g. IL1B, IL2, IL5, IL6, IL8, IL9, IL10, IL12A, IL12B, IL13, IL15, IL15RA, IL17A, IL17C, IL17F, IL19, IL22, IL23A, IL31, IL33), chemokines (e.g. CCL5, CCL17, CCL18, CCL20, CCL22, CCL26, CXCL1, CXCL2, CXCL9, CXCL10), tumour necrosis factors (TNFs) and interferons (IFNs) (e.g. IFN gamma, IFNg). Proinflammatory cytokines are involved in the innate immune system as well as in the adaptive immune system.

Examples of proinflammatory enzymes are matrix metalloproteinase (MMP). MMPs are proteolytic enzymes which degrade extracellular matrix (ECM) proteins and cytokines. An example of a matrix metalloproteinase is matrix metalloproteinase 12 (MMP12). Proinflammatory enzymes may be involved in the innate immune system as well as in the adaptive immune system.

The methods disclosed herein are directed to the treatment of an inflammatory skin condition, such as dermatitis, in a subject. Reference to a “subject” herein mean a human or animal subject. In embodiments, the subject is a human.

A halogenated salicylanilide selected from niclosamide and oxyclozanide (particularly niclosamide) may be administered to the subject in the form of a prodrug of the halogenated salixylanilide. As used herein, the term “prodrug” refers to covalently bonded moiety on the halogenated salixylanilide which modifies the biological and/or physical properties of the compound. The halogenated salixylanilide is released following administration (for example topical administration) of the prodrug compound. Prodrugs may be formed by, for example, modification of a suitable functional group in the parent compound, for example a carboxylic or hydroxy group may be modified to form an ester which is cleaved following topical application of the prodrug. Various prodrug strategies are known and are described in, for example, the following documents:

a) Methods in Enzymology, Vol. 42, p. 309-396, edited by K. Widder, et al. (Academic Press, 1985);

b) Design of Pro-drugs, edited by H. Bundgaard, (Elsevier, 1985);

c) A Textbook of Drug Design and Development, edited by Krogsgaard-Larsen

d) H. Bundgaard, Chapter 5 “Design and Application of Pro-drugs”, by H. Bundgaard p. 113-191 (1991); and

e) H. Bundgaard, Advanced Drug Delivery Reviews, 8, 1-38 (1992).

Unless stated otherwise, reference herein to a “% by weight of a halogenated salicylanilide selected from niclosamide and oxyclozanide (particularly niclosamide), or a pharmaceutically acceptable salt or hydrate thereof” is intended to refer to the amount of the free acid (i.e. non-salt form) of the halogenated salixylanilide. For example, reference to a composition comprising “5% by weight of a halogenated salicylanilide selected from niclosamide and oxyclozanide (particularly niclosamide) or a pharmaceutically acceptable salt thereof” refers to a composition comprising 5% by weight of the halogenated salixylanilide as the free acid. Accordingly, where such a composition comprises a salt of a halogenated salicylanilide selected from niclosamide and oxyclozanide (particularly niclosamide), the absolute amount of the salt of the halogenated salixylanilide in the composition will be higher than 5% by weight in view of the salt counter ion that will be also be present in the composition.

The term “gel” is used herein refers to a semi-solid, apparently homogeneous substance that may be elastic and jelly-like (as in gelatin). The gel comprises a three-dimensional polymeric or inorganic matrix within which is dispersed a liquid phase. The matrix of the gel comprises a network of physically or chemical cross-linked polymers or copolymers that swell but do not dissolve in the presence of a solvent (for example the low molecular weight PEG). The cross-linking within the gel matrix may be physical cross linking (for example by hydrogen bonding or ionic cross-linking) or may be covalently cross-linked. In some embodiments the gel composition is a non-aqueous gel compositions wherein the halogenated salicylanilide selected from niclosamide and oxyclozanide (particularly niclosamide), or a pharmaceutically acceptable salt or hydrate thereof, is dissolved or dispersed in a suitable non-aqueous medium (e.g. PEG). The non-aqueous medium/halogenated salixylanilide solution or dispersion is then dispersed within the polymeric cross-linked network of the gel. Alternatively, the halogenated salicylanilide selected from niclosamide and oxyclozanide (particularly niclosamide), or a pharmaceutically acceptable salt or hydrate thereof, may be dissolved or dispersed within the polymeric cross-linked network of the gel. The gels are preferably clear in appearance; however, turbid gels are also contemplated. Generally, the gel-forming agent, for example gel-forming polymer is present in the gel in an amount of from about 0.5-15% by weight, typically 0.5-2% by weight. The U.S.P. defines gels as a semi-solid system consisting of dispersion made up of either small inorganic particles or large organic molecule enclosing and interpenetrated by liquid.

Reference to a “non-aqueous” composition (e.g. a non-aqueous topical composition), means that the composition is anhydrous and therefore substantially water free. For example, the compositions disclosed herein including the gel, cream and foam compositions contain less than 5%, less than 1% or suitably less than 0.01%, preferably less than 0.001% by weight water. Preferred non-aqueous compositions are those which are anhydrous and contain no detectable water.

Protic organic solvents are those that are capable of hydrogen bonding. The most common examples of protic organic solvents include but are not limited to alcohols and carboxylic acids.

Aprotic organic solvents are those that are not capable of hydrogen bonding. Common aprotic organic solvents include but are not limited to ethers, dimethylformamide (DMF), dimethylsulfoxide (DMSO) and acetonitrile.

Reference to “about” in the context of a numerical is intended to encompass the value+/−10%. For example, about 20% includes the range of from 18% to 22%. Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

The structure of oxyclozanide is shown below.

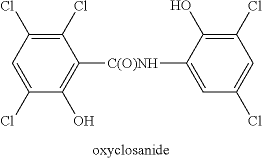

In some embodiments, the halogenated salicylanilide is oxyclozanide or a pharmaceutically acceptable salt or hydrate thereof is used.

In some embodiments, the halogenated salicylanilide is oxyclozanide or a pharmaceutically acceptable salt thereof is used.

In certain embodiments, the halogenated salicylanilide is oxyclozanide.

The structure of niclosamide is shown below.

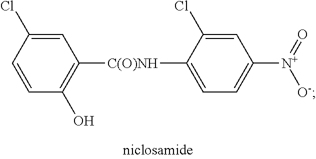

In some embodiments, the halogenated salicylanilide is niclosamide or a pharmaceutically acceptable salt or hydrate thereof.

In some embodiments, the halogenated salicylanilide is niclosamide or a pharmaceutically acceptable salt thereof.

In certain embodiments, the halogenated salicylanilide is niclosamide, i.e. niclosamide in the free acid form.

In certain embodiments the niclosamide is a pharmaceutically acceptable salt of niclosamide, for example an ethanolamine salt, or piperazine salt.

In some embodiments, a hydrate of niclosamide or pharmaceutically acceptable salt thereof is used. However, generally it is preferred that the niclosamide is not administered to the subject in the form of a hydrate.

In certain embodiments the niclosamide is anhydrous niclosamide, or a pharmaceutically acceptable salt thereof.

In a particular embodiment, the niclosamide is anhydrous niclosamide.

A particular problem associated with dermatitis, particularly AD, is pruritus (itching). This symptom of the disease is unpleasant for patients and often results in one or more of stress, anxiety, disturbed sleep, sleep deprivation and psychiatric effects including depression and anxiety, leading to impaired quality of life. Patients are also prone to scratching lesions in an attempt to relieve the pruritus, however, this further damages the already compromised skin of the lesion leading to excoriation, increased erythema, induration and/or swelling. The additional damage to the barrier function of the skin associated with scratching the lesions also enhances exposure to allergens and irritants that can trigger an exacerbation of the dermatitis. Scratching of the lesions also increased the risk of infection of the dermatitis. Accordingly, in embodiments of the invention a halogenated salicylanilide selected from niclosamide and oxyclozanide (particularly niclosamide), or a pharmaceutically acceptable salt or hydrate thereof, is for use in reducing or eliminating pruritus associated with dermatitis (e.g. AD).

The pruritus in a subject may be assessed using a suitable scoring system for the pruritus associated with the dermatitis. For example, a visual analogue scale (VAS) wherein a scale of 0 to 10 is used in which 0=no pruritus, >0-<4 points=mild pruritus, 4-<7 points=moderate pruritus, 7-<9 points=severe pruritus, and 9 points=very severe pruritus (Reich et al Acta Derm. Venereol 2012; 92).

It may be that the treatment of the dermatitis using the a halogenated salicylanilide selected from niclosamide and oxyclozanide (particularly niclosamide), or a pharmaceutically acceptable salt or hydrate thereof, results in a reduction in the pruritus VAS score of 1 point, 2 points, 3 points, 4 points, 5 points, 6 points, 7 points, 8 points or 9 points compared to the VAS score immediately prior to treatment of the subject.

A halogenated salicylanilide selected from niclosamide and oxyclozanide (particularly niclosamide), or a pharmaceutically acceptable salt or hydrate thereof, may be for use in the treatment of mild dermatitis (e.g. mild AD).

A halogenated salicylanilide selected from niclosamide and oxyclozanide (particularly niclosamide), or a pharmaceutically acceptable salt or hydrate thereof, may be for use in the treatment of moderate dermatitis (e.g. moderate AD).

A halogenated salicylanilide selected from niclosamide and oxyclozanide (particularly niclosamide), or a pharmaceutically acceptable salt or hydrate thereof, may be for use in the treatment of severe dermatitis (e.g. severe AD).

A halogenated salicylanilide selected from niclosamide and oxyclozanide (particularly niclosamide), or a pharmaceutically acceptable salt or hydrate thereof, may be for use in the treatment of moderate to severe dermatitis (e.g. moderate to severe AD).

A halogenated salicylanilide selected from niclosamide and oxyclozanide (particularly niclosamide), or a pharmaceutically acceptable salt or hydrate thereof, may be for use in the treatment of mild to moderate dermatitis (e.g. mild to moderate AD).

The severity of the dermatitis may be assessed using known methods. For example, a suitable scoring system that assesses the clinical signs of the dermatitis on the subject. One such scoring method suitable for determining the severity of AD in a human subject is the Total Sign Score (TSS), which includes 6 signs of AD: erythema, edema/papulation, oozing/crusting, excoriation, lichenification and dryness (xerosis). Each sign of the disease is graded using a 4-point scale:
0=absent;
1=mild;
2=moderate; and
3=severe (half point scores are not allowed)

The area of the subject chosen for grading should be representative (i.e. of an average intensity) for each item scored. The individual intensity ratings for each item are then summed together to provide a lesional TSS, which can vary from 0 to 18, wherein the severity of the AD correlates with the magnitude of the TSS.

It may be that the TSS prior to administration of the halogenated salicylanilide selected from niclosamide and oxyclozanide (particularly niclosamide), or a pharmaceutically acceptable salt or hydrate thereof, is greater than or equal to 4, greater than or equal to 5, greater than or equal to 6, greater than or equal to 7, greater than or equal to 8, greater than or equal to 9, greater than or equal to 10, greater than or equal to 12, greater than or equal to 14 or greater than or equal to 16.

It may be that the treatment with the halogenated salicylanilide selected from niclosamide and oxyclozanide (particularly niclosamide), or a pharmaceutically acceptable salt or hydrate thereof, provides a reduction in the TSS of a subject by: 1 point, 2 points, 3 points, 4 points, 5 points, 6 points, 7 points, 8 points, 9 points, 10 points or 15 points compared to the baseline TSS immediately prior to treatment with the halogenated salicylanilide selected from niclosamide and oxyclozanide (particularly niclosamide), or a pharmaceutically acceptable salt or hydrate thereof. The reduction in the size of the TSS is suitably determined by measuring the pre-treatment TSS prior to administration of the halogenated salicylanilide selected from niclosamide and oxyclozanide (particularly niclosamide), or a pharmaceutically acceptable salt or hydrate thereof, and TSS shortly after completion of the treatment with the compound. For example, the TSS is measured within a period of 1 hour to 2 weeks (preferably within a period of 1 hour to 1 week) following completion of the treatment.

The severity of the dermatitis (e.g. AD), may also be assessed using a Target Area Assessment (TAA) which provides a severity grade for a particular lesion to be treated on the subject using a 6 points assessment of:
0=Clear: (no inflammatory signs of atopic dermatitis);
1=Almost clear: (just perceptible erythema, and just perceptible papulation/infiltration);
2=Mild (mild erythema, and mild papulation/infiltration);
3=Moderate: (moderate erythema, and moderate papulation/infiltration);
4=Severe: (severe erythema, and severe papulation/infiltration); and
5=Very severe: (severe erythema, and severe papulation/infiltration with oozing/crusting).

It may be that the base-line TAA of a subject prior to treatment with a halogenated salicylanilide selected from niclosamide and oxyclozanide (particularly niclosamide), or a pharmaceutically acceptable salt or hydrate thereof, is greater than or equal to 1; greater than or equal to 2; greater than or equal to 3; greater than or equal to 4; or is 5.

It may be that the treatment with a halogenated salicylanilide selected from niclosamide and oxyclozanide (particularly niclosamide), or a pharmaceutically acceptable salt or hydrate thereof, provides a reduction in the TAA of a subject by 1 point, 2 points, 3 points, 4 points or 5 points compared to the baseline TAA immediately prior to treatment with a halogenated salicylanilide selected from niclosamide and oxyclozanide (particularly niclosamide), or a pharmaceutically acceptable salt or hydrate thereof.

Other scoring systems may also be used to assess the efficacy of the treatment on the dermatitis (e.g. AD). These include the SCORAD index, the Eczema Area and Severity Index (EASI), Investigator's Global Assessment (IGA) and the Patient-Oriented Eczema Measure (POEM) severity scale (Eichenfield et al. Guidelines of care for the management of atopic dermatitis: section 1. Diagnosis and assessment of atopic dermatitis. J Am Acad. Dermatol. 2014 February; 70(2):338-51).

In embodiments the severity of the dermatitis is assessed using the IGA score. This is a five point scale:
0: Clear (no inflammatory signs)
1: Almost clear (just perceptible erythema, just perceptible papulation/infiltration)
2: Mild disease (mild erythema and mild papulation/infiltration)
3: Moderate disease (moderate erythema, moderate papulation/infiltration)
4: Severe disease (severe erythema, severe papulation/infiltration)

It may be that the baseline IGA of a subject prior to treatment with a halogenated salicylanilide selected from niclosamide and oxyclozanide (particularly niclosamide), or a pharmaceutically acceptable salt or hydrate thereof, is greater than or equal to 1; greater than or equal to 2; greater than or equal to 3; or is 4.

It may be that the treatment with a halogenated salicylanilide selected from niclosamide and oxyclozanide (particularly niclosamide), or a pharmaceutically acceptable salt or hydrate thereof, provides a reduction in the IGA score of a subject by 1 point, 2 points, 3 points or 4 points compared to the baseline IGA score immediately prior to treatment with the halogenated salicylanilide selected from niclosamide and oxyclozanide (particularly niclosamide), or a pharmaceutically acceptable salt or hydrate thereof.

In embodiments the severity of the dermatitis (e.g. AD) is assessed using the Eczema Area and Severity Index (EASI). The EASI provides a composite score ranging from 0 to 72 that takes into account the degree of erythema, induration/infiltration (papules), excoriation, and lichenification for each of four body regions, with adjustment for the percentage of body surface area (BSA) involved for each body region and for the proportion of the body region to the whole body.

In the EASI scoring system four anatomic sites (head, upper extremities, trunk, and lower extremities) are assessed for erythema, induration/infiltration (papules), excoriation, and lichenification as seen on the day of the examination. The severity of each sign is assessed using a 4-point scale (half steps are allowed in the scoring):
0=None
1=Mild
2=Moderate
3=Severe

The area affected by dermatitis within a given anatomic site is estimated as a percentage of the total area of that anatomic site and assigned a numerical value according to the degree of atopic dermatitis involvement as follows:
0=No involvement
1=<10%
2=10% to <30%
3=30% to <50%
4=50% to <70%
5=70% to <90%
6=90% to 100%

The EASI score is obtained by using the formula:

EASI=0.1(Eₕ+Iₕ+Exₕ+Lₕ)Aₕ+0.2(Eᵤ+Iᵤ+Exᵤ+Lᵤ)Aᵤ+0.3(Eₜ+Iₜ+Exₜ+Lₜ)Aₜ+0.4(Eₗ+Iₗ+Exₗ+Lₗ)Aₗ

wherein E, I, Ex, L, and A denote erythema, induration, excoriation, lichenification and area, respectively, and h, u, t, and l denote head, upper extremities, trunk, and lower extremities, respectively (see Tofte et al. J. Eur. Acad. Dermatol. Venereol. 1998; 11 (suppl. 2):S197)

It may be that the Baseline EASI score of the subject prior to treatment with a halogenated salicylanilide selected from niclosamide and oxyclozanide (particularly niclosamide), or a pharmaceutically acceptable salt or hydrate thereof, is greater than or equal to 5, greater than or equal to 10, greater than or equal to 15, greater than or equal to 20, greater than or equal to 25, greater than or equal to 30, greater than or equal to 35, greater than or equal to 40, greater than or equal to 45, greater than or equal to 50, greater than or equal to 55, greater than or equal to 60 or greater than or equal to 65.

It may be that the treatment a halogenated salicylanilide selected from niclosamide and oxyclozanide (particularly niclosamide), or a pharmaceutically acceptable salt or hydrate thereof, provides a reduction in the EASI Score of a subject by at least: 1 point, 2 points, 3 points, 4 points, 5 points, 6 points, 7 points, 8 points, 9 points, 10 points, 15 points, 20 points, 25 points, 30 points, 35 points, 40 points, 45 points, 50 points, 55 points or 60 points compared to the baseline EASI Score prior to treatment with a halogenated salicylanilide selected from niclosamide and oxyclozanide (particularly niclosamide), or a pharmaceutically acceptable salt or hydrate thereof.

The type of dermatitis affecting the subject may be readily determined by a physician using well-known diagnostic methods. In the case of AD subjects may, for example, be diagnosed using the Hanifin & Rajka criteria (Hanifin & Rajka “Diagnostic feature of atopic dermatitis”, Acta Derm. Ven. vol 92, (suppl): 44-47, 1980). The criteria for AD are summarised below.

Major Criteria (patient must have at least three)
Pruritus
Typical morphology and distribution:
   Adults: flexural lichenification or linearity
   Children and infants: involvement of facial and extensor surfaces
Chronic or chronically relapsing dermatitis
Personal or family history of atopy (asthma, allergic rhinitis, atopic dermatitis)
   Minor Criteria (patient must have at least three)
Xerosis
Ichthyosis/keratosis pilaris/palmar hyperlinearity
Immediate (Type 1) skin test reactivity
Elevated serum IgE
Early age at onset
Tendency to skin infections (Staphylococcus aureus, herpes simplex)/impaired cellular immunity
Tendency to nonspecific hand/foot dermatitis
Nipple eczema
Cheilitis
Recurrent conjunctivitis
Dennie-Morgan infraorbital fold
Keratoconus
Anterior subcapsular cataracts
Orbital darkening
Facial pallor/erythema
Pityriasis alba
Anterior neck folds
Itch when sweating
Intolerance to wool and lipid solvents
Perifollicular accentuation
Food intolerance
Course influenced by environmental/emotional factors
White demographic/delayed blanch

It may be that a halogenated salicylanilide selected from niclosamide and oxyclozanide (particularly niclosamide), or a pharmaceutically acceptable salt or hydrate thereof, improves, eliminates or prevents one or more of the major and/or minor dermatitis criteria above.

It may be that a halogenated salicylanilide selected from niclosamide and oxyclozanide (particularly niclosamide), or a pharmaceutically acceptable salt or hydrate thereof, is used for treatment of atopic dermatitis in a subject and the subject has a condition selected from asthma, rhinitis and a food allergy in addition to atopic dermatitis.

In animals, a scoring method suitable for determining the severity of AD is the Canine Atopic Dermatitis Extent and Severity Index (CADESI), for example CADESI-01, CADESI-02 or CADESI-03 (Olivry et al. Validation of CADESI-03, a severity scale for clinical trials enrolling dogs with atopic dermatitis. Veterinary Dermatology, 18: 78-86), or CADESI-04 (Olivry T et al, Vet Dermatol. 2014 April; 25(2):77-85). CADESI-4 is currently recommended by ICADA (International Committee on Allergic Diseases of Animals) and is a preferred scoring system for AD in animals. These scoring systems may also be used to grade other, similar forms of dermatitis in animals.

The CADESI scores quantitatively describe the dog's skin condition, separately scoring areas of a dog's body for erythema, lichenification, and/or excoriation as ‘Normal or absent’ (0), ‘Mild’ (1), ‘Moderate’ (2), or ‘Severe’ (3). CADESI-03 differs from CADESI-02 in that it has an increased number of body sites assessed from 40 to 62, and includes another clinical sign (self-induced alopecia) and each sign is graded in a wider scale (scale of 0 to 5). CADESI-03. CADESI-04 requires only 20 defined body sites and takes approximately 33% of the time to conduct as compared to CADESI-03. Accordingly, a preferred dermatitis scoring system for animals is CADESI-04.

It may be that the CADESI score (e.g. CADESI-03 or preferably CADESI-04 score) is reduced by 2, 4, 6 or 8 points compared to the score immediately before commencing treatment (the baseline score) of the animal.

AD is characterised by an acute phase and a chronic phase. Acute AD is thought to be predominantly driven by Th2, whereas there is a switch to Th1 in the chronic stages of the disease (Gittler et al. J Allergy Clin Immunol. 2012 December; 130(6): 1344-1354) Acute AD lesions are typically bright red, “wet” and flat, becoming dull red, dry and thick with chronicity.

A halogenated salicylanilide selected from niclosamide and oxyclozanide (particularly niclosamide), or a pharmaceutically acceptable salt or hydrate thereof, may be for use in the treatment of acute form of dermititis (e.g. acute AD). For example, a halogenated salicylanilide selected from niclosamide and oxyclozanide (particularly niclosamide), or a pharmaceutically acceptable salt or hydrate thereof, may be for use in the treatment or prevention of lesion redness (erythema, inflammation), induration, papulation, pruritus or excoriation in a patient with acute AD. The acute AD may be mild, moderate or severe acute AD, for example moderate to severe acute AD or mild to moderate AD.

A halogenated salicylanilide selected from niclosamide and oxyclozanide (particularly niclosamide), or a pharmaceutically acceptable salt or hydrate thereof, may be for use in the treatment of a chronic form of dermatitis (e.g. chronic AD). For example, a halogenated salicylanilide selected from niclosamide and oxyclozanide (particularly niclosamide), or a pharmaceutically acceptable salt or hydrate thereof, may be for use in the treatment or prevention of lichenification (for example, lined skin or prurigo nodules), pruritus or excoriation in a subject with chronic AD. The chronic AD may be mild, moderate or severe chronic AD, for example moderate or severe chronic AD.

The inflammation and scratching of dermatitis lesions can result in hyperpigmentation or hypopigmentation of the skin. This can be present even after the inflammation has resolved and the dermatitis is in remission. It may be that the niclosamide, or a pharmaceutically acceptable salt or hydrate thereof, is for use in the treatment or prevention of skin hyperpigmentation associated with dermatitis (e.g. AD). In other embodiments a halogenated salicylanilide selected from niclosamide and oxyclozanide (particularly niclosamide), or a pharmaceutically acceptable salt or hydrate thereof, is for use in the treatment or prevention of hypopigmentation associated with dermatitis (e.g. AD).

It may be that the dermatitis lesions are colonized by bacteria, for example the lesion may be colonized by Gram-positive bacteria. In certain embodiments a halogenated salicylanilide selected from niclosamide and oxyclozanide (particularly niclosamide), or a pharmaceutically acceptable salt or hydrate thereof, is for use in the treatment of a dermatitis lesion (e.g. an AD lesion) that is colonized by Gram-positive bacteria. The Gram-positive bacteria that may colonize the lesion include, but are not limited to Staphylococcus spp., Streptococcus spp. or Propionibacterium spp. The Gram-positive bacteria may be a Staphylococcus spp. or Streptococcus spp. The Gram-positive bacteria may be selected from Staphylococcus aureus or Streptococcus pyogenes. The bacteria may be resistant to conventional antibiotic agents. For example, the bacteria may be a MRSA strain.

In other embodiments the dermatitis lesion is not colonized by bacteria. Reference to “not colonized” means that the lesion is substantially free from bacteria, for example the lesion to be treated in the subject carries less than 1000 CFU/cm². The CFU in a sample taken from the lesion may be determined using conventional cell culturing methods. The sample could be, for example, a swab or skin biopsy obtained from the lesion.

In embodiments, the halogenated salicylanilide selected from niclosamide and oxyclozanide (particularly niclosamide), or a pharmaceutically acceptable salt or hydrate thereof, is for use in the treatment of dermatitis (e.g. AD) that is not colonized or infected by bacteria, for example the AD lesion is not colonized or infected with a Gram-positive bacteria.

In embodiments, the inflammatory skin condition is a clinically uninfected inflammatory skin condition, such as a clinically uninfected atopic dermatitis.

Subjects with certain forms of dermatitis, including AD, are prone to exacerbation (flares) in their dermatitis. In the case of AD a flare could result from, for example, exposure to an irritant or allergen or a change in ambient conditions such as elevated temperature or humidity. Accordingly, a halogenated salicylanilide selected from niclosamide and oxyclozanide (particularly niclosamide), or a pharmaceutically acceptable salt or hydrate thereof, may be useful in the prevention or treatment of exacerbations of dermatitis (e.g. AD) in a subject. It may be that a halogenated salicylanilide selected from niclosamide and oxyclozanide (particularly niclosamide), or a pharmaceutically acceptable salt or hydrate thereof, is for use in reducing the frequency of exacerbations of dermatitis (e.g. AD) in a subject. It may be that a halogenated salicylanilide selected from niclosamide and oxyclozanide (particularly niclosamide), or a pharmaceutically acceptable salt or hydrate thereof, is for use in reducing the severity of an exacerbation of dermatitis (e.g. AD) in a subject. It may be that a halogenated salicylanilide selected from niclosamide and oxyclozanide (particularly niclosamide), or a pharmaceutically acceptable salt or hydrate thereof, is for use in reducing the duration of an exacerbation of dermatitis (e.g. AD) in a subject.

Accordingly, in embodiments a halogenated salicylanilide selected from niclosamide and oxyclozanide (particularly niclosamide), or a pharmaceutically acceptable salt or hydrate thereof, is for use in the treatment of an exacerbation of dermatitis (e.g. AD). In embodiments a halogenated salicylanilide selected from niclosamide and oxyclozanide (particularly niclosamide), or a pharmaceutically acceptable salt or hydrate thereof, is for use in preventing or reducing the frequency of dermatitis (e.g. AD) exacerbations in a subject. In embodiments a halogenated salicylanilide selected from niclosamide and oxyclozanide (particularly niclosamide), or a pharmaceutically acceptable salt or hydrate thereof, is for use in reducing the severity of exacerbations of dermatitis (e.g. AD) in a subject.

In the embodiments described herein that refer to exacerbations of the dermatitis, the exacerbation may be an exacerbation of one or more of the symptoms of the dermatitis described herein (e.g. an exacerbation of one or more of pruritus, erythema, induration or excoriation).

In some embodiments the subject is a paediatric human patient, for example a patient less than 18 years old. The patient may be less than 17, less than 16, less than 15, less than 14, less than 13, less than 10 or less than 5 years old. For example, the patient may be from 6 months to 18 years old, from 1 to 18 years old, from 2 to 18 years old, from 2 to 16 years old, from 3 to 18 years old, from 4 to 18 years old, from 5 to 18 years old or from 5 to 16 years old.

In some embodiments the subject is an adult human, for example a human aged 18 or older.

### Pharmaceutical Compositions

A halogenated salicylanilide selected from niclosamide and oxyclozanide (particularly niclosamide), or a pharmaceutically acceptable salt or hydrate thereof, is suitably administered to the subject in the form of a pharmaceutical composition comprising the halogenated salicylanilide selected from niclosamide and oxyclozanide (particularly niclosamide), or a pharmaceutically acceptable salt or hydrate thereof, and a pharmaceutically acceptable excipient.

Conventional procedures for the selection and preparation of suitable pharmaceutical compositions are described in, for example, “Pharmaceuticals—The Science of Dosage Form Designs”, M. E. Aulton, Churchill Livingstone, 1988.

A halogenated salicylanilide selected from niclosamide and oxyclozanide (particularly niclosamide), or a pharmaceutically acceptable salt or hydrate thereof, is suitably compounded with an appropriate and convenient amount of excipients which may vary from about 5 to about 99 percent by weight of the total composition. The compositions may be prepared using conventional procedures well known in the art.

A halogenated salicylanilide selected from niclosamide and oxyclozanide (particularly niclosamide), or a pharmaceutically acceptable salt or hydrate thereof, is topically administered to the subject.

In embodiments, a halogenated salicylanilide selected from niclosamide and oxyclozanide (particularly niclosamide), or a pharmaceutically acceptable salt or hydrate thereof, is topically administered to a subject for the treatment of dermatitis (e.g. AD). Suitably a halogenated salicylanilide selected from niclosamide and oxyclozanide (particularly niclosamide), or a pharmaceutically acceptable salt or hydrate thereof, is topically administered directly to an AD lesion on the subject.

When the halogenated salicylanilide selected from niclosamide and oxyclozanide (particularly niclosamide), or a pharmaceutically acceptable salt or hydrate thereof, is topically administered it is suitably administered in the form of a pharmaceutical composition in a dosage form suitable for topical administration, for example as a topical cream, ointment, gel, paste, lotion, foam, or aqueous, non-aqueous or oily solution or suspension.

In particular embodiments the halogenated salicylanilide selected from niclosamide and oxyclozanide (particularly niclosamide), or a pharmaceutically acceptable salt or hydrate thereof, is formulated as a non-aqueous pharmaceutical composition suitable for topical administration, for example a non-aqueous cream, ointment, gel, paste, lotion, or foam comprising the halogenated salicylanilide selected from niclosamide and oxyclozanide (particularly niclosamide), or a pharmaceutically acceptable salt or hydrate thereof.

In some embodiments the halogenated salicylanilide selected from niclosamide and oxyclozanide (particularly niclosamide), or a pharmaceutically acceptable salt or hydrate thereof, is formulated as an aqueous pharmaceutical composition suitable for topical administration, for example an aqueous cream, ointment, gel, paste, lotion, or foam comprising the halogenated salicylanilide selected from niclosamide and oxyclozanide (particularly niclosamide) or a pharmaceutically acceptable salt or hydrate thereof.

In certain embodiments the halogenated salicylanilide selected from niclosamide and oxyclozanide (particularly niclosamide), or a pharmaceutically acceptable salt or hydrate thereof, is formulated as a topical composition comprising the halogenated salicylanilide selected from niclosamide and oxyclozanide (particularly niclosamide), or a pharmaceutically acceptable salt thereof of hydrate thereof; and polyethylene glycol (PEG).

In some embodiments the topical composition is an aqueous topical composition comprising the halogenated salicylanilide selected from niclosamide and oxyclozanide (particularly niclosamide) or pharmaceutically acceptable salt or hydrate thereof. The aqueous topical composition suitably comprises at least 5% by weight of water and one or more pharmaceutically acceptable excipients.

In other embodiments the topical composition is a non-aqueous topical composition comprising the halogenated salicylanilide selected from niclosamide and oxyclozanide (particularly niclosamide) or a pharmaceutically acceptable salt or hydrate thereof.

The topical composition may be in any form suitable for topical administration, for example a cream, ointment, gel, paste, lotion, foam, or aqueous, non-aqueous or oily solution or suspension comprising the halogenated salicylanilide selected from niclosamide and oxyclozanide (particularly niclosamide).

In some embodiments the topical composition may be in the form of an aqueous or non-aqueous gel comprising the halogenated salicylanilide selected from niclosamide and oxyclozanide (particularly niclosamide), or a pharmaceutically acceptable salt or hydrate thereof, and a gel forming agent. In some embodiments the topical composition may be in the form of a non-aqueous gel comprising the halogenated salicylanilide selected from niclosamide and oxyclozanide (particularly niclosamide), or a pharmaceutically acceptable salt or hydrate thereof, and a gel forming agent. The gel forming agent may be any suitable gel-forming agent, including, but not limited to any of the gel forming agents described herein. In some embodiments the topical composition may be in the form of an aqueous cream or ointment comprising the halogenated salicylanilide selected from niclosamide and oxyclozanide (particularly niclosamide), or a pharmaceutically acceptable salt or hydrate thereof, and a suitable aqueous cream or non-aqueous ointment base.

In some embodiments the topical composition may be in the form of a non-aqueous cream or ointment comprising the halogenated salicylanilide selected from niclosamide and oxyclozanide (particularly niclosamide), or a pharmaceutically acceptable salt or hydrate thereof, and a suitable non-aqueous cream or non-aqueous ointment base.

The topical composition may be prepared using known carriers or “bases” in which the halogenated salicylanilide selected from niclosamide and oxyclozanide (particularly niclosamide), or a pharmaceutically acceptable salt or hydrate thereof, is dissolved or dispersed. In some embodiments, the halogenated salicylanilide selected from niclosamide and oxyclozanide (particularly niclosamide), or a pharmaceutically acceptable salt or hydrate thereof, is dissolved or partially dissolved in the topical composition. For example, the topical composition may comprise the halogenated salicylanilide selected from niclosamide and oxyclozanide (particularly niclosamide), or a pharmaceutically acceptable salt or hydrate thereof, dissolved or dispersed in a suitable base formulation selected from an oleaginous base (e.g. petrolatum, white petrolatum, yellow ointment or white ointment), an absorption base (e.g. hydrophilic petrolatum or lanolin), a water-removable base (oil in water emulsion); a water-soluble base (e.g. a polyethylene glycol).

In certain embodiments the halogenated salicylanilide selected from niclosamide and oxyclozanide (particularly niclosamide), or a pharmaceutically acceptable salt or hydrate thereof, is formulated as a topical composition comprising the halogenated salicylanilide selected from niclosamide and oxyclozanide (particularly niclosamide), or a pharmaceutically acceptable salt or hydrate thereof, and a non-polymeric glycol (for example an alkylene glycol, e.g. a C₂₋₈ alkylene glycol such as propylene glycol).

In certain embodiments the halogenated salicylanilide selected from niclosamide and oxyclozanide (particularly niclosamide), or a pharmaceutically acceptable salt or hydrate thereof, is formulated as a topical composition comprising the halogenated salicylanilide selected from niclosamide and oxyclozanide (particularly niclosamide), or a pharmaceutically acceptable salt or hydrate thereof, and a glycol ether, for example 2-(2-ethoxyethoxy)ethanol (Transcutol).

In certain embodiments the halogenated salicylanilide selected from niclosamide and oxyclozanide (particularly niclosamide), or a pharmaceutically acceptable salt or hydrate thereof, is formulated as a non-aqueous topical composition comprising:
(i) the halogenated salicylanilide selected from niclosamide and oxyclozanide (particularly niclosamide), or a pharmaceutically acceptable salt or hydrate thereof; and
(ii) polyethylene glycol (PEG).

In certain embodiments the halogenated salicylanilide selected from niclosamide and oxyclozanide (particularly niclosamide), or a pharmaceutically acceptable salt or hydrate thereof, is formulated as a non-aqueous topical gel composition comprising the halogenated salicylanilide selected from niclosamide and oxyclozanide (particularly niclosamide), or a pharmaceutically acceptable salt or hydrate thereof, and a gel forming agent. The gel-forming agent may be any of the gel-forming agents disclosed herein. Suitably the topical gel composition further comprises a PEG.

Suitably the PEG in the composition is selected such that the composition together with any other components of the composition (e.g. in the form of a liquid, semi-solid or gel composition) can easily be applied to, spread over and/or rubbed into the skin. It may be that the PEG has a melting point that is less than 40° C. or less than 35° C. In certain embodiments the PEG is selected such that it is soft or, suitably molten at body temperature. For example, the PEG may have a melting point of 32° C. or less, or less than 30° C., or less than 25° C.

It may be that the halogenated salicylanilide selected from niclosamide and oxyclozanide (particularly niclosamide), or a pharmaceutically acceptable salt or hydrate thereof, is present in an amount of up to 10% by weight of the composition, for example from 0.01% to 4.5% by weight of the composition, from 0.05% to 4.5% by weight, 0.1% to 3% by weight, from 1% to 3% by weight, from 1.5% to 4.5% by weight. For example, at about 2% by weight of the composition or at about 4% by weight of the composition.

It may be that the topical composition comprising the halogenated salicylanilide selected from niclosamide and oxyclozanide (particularly niclosamide), or a pharmaceutically acceptable salt or hydrate thereof, provides a local pH of greater than 4.5 at the site of application of the composition (for example an AD lesion). It may be that the composition provides a local pH of less than 6 at the site of application following topical application of the composition. Suitably the composition provides a local pH in the range of from about 4.5 to about 6 at the site of topical application of the composition.

### Non-Aqueous Topical Compositions

In particular embodiments the halogenated salicylanilide selected from niclosamide and oxyclozanide (particularly niclosamide), or a pharmaceutically acceptable salt or hydrate thereof, is formulated as a non-aqueous pharmaceutical composition suitable for topical administration. For example, a non-aqueous cream, ointment, gel or foam comprising the halogenated salicylanilide selected from niclosamide and oxyclozanide (particularly niclosamide) or a pharmaceutically acceptable salt or hydrate thereof.

In certain embodiments the non-aqueous topical composition comprises:
(i) the halogenated salicylanilide selected from niclosamide and oxyclozanide (particularly niclosamide), or a pharmaceutically acceptable salt or hydrate thereof; and
(ii) polyethylene glycol (PEG), preferably a PEG with a melting point of less than 40° C.

In certain embodiments the non-aqueous composition comprises:
(i) the halogenated salicylanilide selected from niclosamide and oxyclozanide (particularly niclosamide), or a pharmaceutically acceptable salt or hydrate thereof; and
(ii) greater than 60% by weight of a PEG, preferably wherein the average molecular weight of the PEG is 800 or less and particularly 600 or less. For example, the average molecular weight of the PEG is less than 800. It may be that the average molecular weight of the PEG is less than 400.

In certain embodiments, the composition further comprises a non-polymeric glycol (for example an alkylene glycol, e.g. a C₂₋₈ alkylene glycol, preferably a 02-6 alkylene glycol and especially propylene glycol).

In certain embodiments the non-aqueous topical composition comprises propylene glycol. Accordingly the composition may comprise:
(i) the halogenated salicylanilide selected from niclosamide and oxyclozanide (particularly niclosamide), or a pharmaceutically acceptable salt or hydrate thereof;
(ii) polyethylene glycol (PEG), (preferably a PEG with a melting point of less than 40° C.); and
(iii) a C₂₋₈ alkylene glycol (preferably propylene glycol).

In certain embodiments the non-aqueous topical composition comprises:
(i) 0.1 to 5% by weight of the halogenated salicylanilide selected from niclosamide and oxyclozanide (particularly niclosamide), or a pharmaceutically acceptable salt or hydrate thereof;
(ii) polyethylene glycol (PEG) with a melting point of less than 40° C.; and
(iii) 0.5 to 30% (for example 5 to 25%) by weight of a non-polymeric glycol (preferably propylene glycol).

Examples of PEG, preferably with an average molecular weight of less than 600, which may be used in the non-aqueous composition are described in more detail below under the section “Polyethylene Glycol (PEG)”

It may be that the non-aqueous composition comprises up to 10%, up to 20%, up to 30%, up to 35%, up to 40%, up to 45%, up to 50% or up to 55% by weight of PEG. For example, wherein the lower limit of PEG is 1% by weight and the upper limit is any of the values set out in this paragraph. For example, wherein the lower limit of PEG is 5% by weight and the upper limit is any of the values set out in this paragraph (e.g. a range of 5% to 20, 30, 40, 50, 60, 70, 80, 90 or 95% by weight PEG).

In some embodiments it has been found that a high concentration of PEG in the composition provides a non-aqueous topical composition with advantageous properties, for example one or more of improved dermal penetration and/or good tolerability when topically applied to the skin. Certain compositions described herein provide high concentration of the halogenated salicylanilide selected from niclosamide and oxyclozanide (particularly niclosamide) in skin tissues (e.g. the dermis and epidermis) and very low levels of systemic exposure (e.g. in the plasma) to the halogenated salicylanilide selected from niclosamide and oxyclozanide (particularly niclosamide). The compositions are therefore expected to provide an effective local topical treatment of, for example, a dermal condition, with little or no systemic side-effects, because the systemic exposure is low. Such compositions are expected to provide a wide therapeutic window between the beneficial therapeutic effects and the onset of undesirable systemic side effects that may be associated with the halogenated salicylanilide selected from niclosamide and oxyclozanide (particularly niclosamide). Such side effects could be systemic toxicity.

It may be that the non-aqueous composition comprises more than 65%, more than 70%, more than 75%, more than 80%, more than 85%, more than 90%, more than 95%, more than 96%, more than 97%, more than 98% or more than 99% PEG (preferably with an average molecular weight of 600 or less, for example a PEG with an average molecular weight of 400 or less); and wherein the % is by weight of the composition. Further amount of the PEG which may be present in the composition are described under the section “Polyethylene Glycol (PEG)”.

It may be that the halogenated salicylanilide selected from niclosamide and oxyclozanide (particularly niclosamide), or a pharmaceutically acceptable salt or hydrate thereof, is present in the non-aqueous composition in an amount of 0.01% to 10%, for example from 0.01% to 5%, from 0.01% to 4.5%, from 0.01% to 4%, from 0.01% to 3.5%, from 0.01% to 3%, from 0.1% to 5%, from 0.1% to 4.5%, from 0.1% to 4%, from 0.1% to 3.5%, from 0.1 to 3%, from 0.1 to 2.5%, from 0.1 to 2%, from 0.1 to 1.5%, from 0.1 to 1%, or from 0.5 to 3%, for example about 1%, about 2% about 2.5% about 3%, about 4%, about 4.5% or about 5%, wherein the % are by weight based upon the weight of the composition. The halogenated salicylanilide selected from niclosamide and oxyclozanide (particularly niclosamide) may be in the form of a hydrate, however, this is less preferred in the non-aqueous compositions described herein. Accordingly, it is preferred that the halogenated salicylanilide selected from niclosamide and oxyclozanide (particularly niclosamide) is in a substantially anhydrous form.

It may be that the non-aqueous composition of the invention comprises:
(i) 0.01 to 4.5% (e.g. 0.1 to 4%, or 0.1 to 3.5, or 0.1 to 3% or about 2%, or about 4%) by weight of the halogenated salicylanilide selected from niclosamide and oxyclozanide (particularly niclosamide), or a pharmaceutically acceptable salt or hydrate thereof; and
(ii) at least 70% (for example at least 90%) by weight of a PEG, wherein the average molecular weight of the PEG is 600 or less (for example less than 600 or from about 200 to about 600 or about 400).

It may be that the non-aqueous compositions described herein further comprise a polar organic solvent for example a polar organic solvent selected from an alkylene glycol (e.g. propylene glycol), 2-(2-ethoxyethoxy)ethanol, glycerol, a macrogol stearyl ether (e.g. macrogol 15 stearyl ether) or a macrogol isostearate or a fatty alcohol, for example a C₁₂-C₁₈-alcohol such as cetostearyl alcohol or a mixture two or more thereof. It may be that the polar organic is present in the composition in an amount of from about 5% to about 65%, about 10% to about 55% or about 25% to about 50% by weight of the composition.

It may be that the non-aqueous compositions described herein further comprise a glycol, for example an alkylene glycol (e.g. propylene glycol). It may be that the composition comprises from about 5% to about 30%, about 10% to about 30%, or about 14% to about 28% by weight of a glycol, particularly propylene glycol.

It may be that the non-aqueous compositions described herein further comprise 2-(2-ethoxyethoxy)ethanol. It may be that the composition comprises from about 1% to about 25%, about 5% to about 20% or about 10% to about 20% by weight of 2-(2-ethoxyethoxy)ethanol.

It may be that the non-aqueous compositions described herein further comprise glycerol. It may be that the composition comprises from about 5% to about 30%, about 10% to about 30%, or about 15% to 25% by weight of glycerol.

It may be that the composition comprises one or more non-polar excipients, for example one or more non-polar oils, hydrocarbon solvents or waxes. It may be that the composition comprises one or more non-polar excipients selected from aromatic or aliphatic esters, a mineral oil, a vegetable oil and long-chain or medium chain triglycerides. For example, the non-polar excipients may be selected from one or more of a mineral oil, (e.g. liquid paraffin or a paraffin wax) and medium chain triglycerides. It may be that the non-polar excipients are present in the composition in an amount of from about 2% to about 50%, about 5% to about 40%, about 5% to about 30%, or about 5% to 25% by weight of the composition.

It may be that the non-aqueous compositions described herein further comprise one or more surfactant or emulsifiers, for example an ionic or non-ionic surfactant or emulsifiers. Representative examples of surfactants or emulsifiers include any of those described herein, for example a PEGylated fatty acid glyceride (labrasol), polyoxyethylene glycol sorbitan alkyl ester (polysorbate), a polyoxyethylene glycol alkyl ether (Brij), polyoxyethylene ethers of fatty alcohols (ceteareth), or a fatty acid ester of glycerol (e.g. glyceryl stearate). It may be that the surfactant or emulsifiers are present in the composition in an amount of from about 0.1% to about 15%, about 0.2% to about 10%, or about 0.2% to about 5% by weight of the composition.

In certain embodiments the non-aqueous composition comprises a non-aqueous emulsion or microemulsion. Non-aqueous emulsion or microemulsion compositions are particularly suitable for providing compositions in the form of a non-aqueous topical cream composition. The non-aqueous emulsion comprises a non-aqueous hydrophilic phase (suitably comprising polar excipients) and a non-aqueous hydrophobic phase which is immiscible with the hydrophilic phase (suitably comprising non-polar excipients such as an oil). It may be that the hydrophilic phase comprises the continuous phase of the emulsion and the hydrophobic phase is dispersed within the hydrophilic phase as the discontinuous phase of the emulsion. In certain embodiments the non-aqueous hydrophobic phase comprises the continuous phase of the emulsion and the non-aqueous phase is dispersed within the non-aqueous hydrophobic phase as the discontinuous phase of the emulsion.

In certain embodiments the non-aqueous hydrophilic phase comprises the halogenated salicylanilide selected from niclosamide and oxyclozanide (particularly niclosamide), the PEG and optionally one or more of the polar solvents described herein. Accordingly it may be that the non-aqueous hydrophilic phase comprises the halogenated salicylanilide selected from niclosamide and oxyclozanide (particularly niclosamide), PEG and optionally one or more polar solvents selected from propylene glycol, 2-(2-ethoxyethoxy)ethanol, glycerol, a macrogol stearyl ether (e.g. macrogol 15 stearyl ether) and a fatty alcohol, for example a C₁₂-C₁₈-alcohol such as cetostearyl alcohol.

It may be that the non-aqueous hydrophobic phase of the emulsion or microemulsion comprises one or more of the non-polar excipients described herein, for example, a mineral oil, a vegetable oil and long-chain or medium chain triglycerides.

In those embodiments where the composition is in the form of a non-aqueous emulsion or microemulsion the composition suitably comprises a surfactant or emulsifier, for example one or more of the surfactants or emulsifiers described herein.

Suitably the non-aqueous composition comprises a solution of the halogenated salicylanilide selected from niclosamide and oxyclozanide (particularly niclosamide). Accordingly, it is preferred that the halogenated salicylanilide selected from niclosamide and oxyclozanide (particularly niclosamide) is completely dissolved in the non-aqueous composition. However, it is contemplated that the halogenated salicylanilide selected from niclosamide and oxyclozanide (particularly niclosamide), or a pharmaceutically acceptable salt or hydrate thereof, may present as a dispersion in the composition. Alternatively, in some embodiments at least a proportion of the halogenated salicylanilide selected from niclosamide and oxyclozanide (particularly niclosamide), or a pharmaceutically acceptable salt or hydrate thereof, is dissolved in the composition. In this embodiment it is preferred that at least 80%, preferably at least 90%, more preferably at least 95% by weight of the halogenated salicylanilide selected from niclosamide and oxyclozanide (particularly niclosamide) is dissolved in the composition.

### Non-Aqueous Gel Compositions

In certain embodiments the non-aqueous topical composition of the invention is in the form of a non-aqueous topical gel composition

In certain embodiments there is provided a non-aqueous topical gel composition comprising:
(i) the halogenated salicylanilide selected from niclosamide and oxyclozanide (particularly niclosamide), or a pharmaceutically acceptable salt or hydrate thereof; and
(ii) PEG with a melting point of less than 40° C.; and
(iii) a gel forming agent.

In certain embodiments there is provided a non-aqueous topical gel composition comprising:
(i) the halogenated salicylanilide selected from niclosamide and oxyclozanide (particularly niclosamide), or a pharmaceutically acceptable salt or hydrate thereof;
(ii) greater than 60% by weight of a PEG, preferably wherein the average molecular weight of the PEG is less than 600; and
(iii) a gel-forming agent.

Particular aspects of the non-aqueous gel compositions are described below.

### Gel-Forming Agent

It may be that the gel-forming agent present in the compositions disclosed herein is an inorganic gel-forming agent. It may be that the gel-forming agent is a gel-forming polymer.

### Inorganic Gel Forming Agents

It may be that the gel-forming agent is an inorganic gel-forming agent, for example a bentonite or a silica. It may be that the gel-forming agent is magnesium aluminium silicate (Veegum®).

### Gel-Forming Polymers

The gel-forming agent may be a gel-forming polymer. The gel-forming polymer may be a hydrophilic gel-forming polymer. The gel-forming polymer may be selected from the group consisting of: gelatin; agar; agarose; pectin; carrageenan; chitosan; alginate; starch; starch components (e.g. amylose or amylopectin); tragacanth gum; xanthan gum; gum Arabic (acacia gum); guar gum; gellan gum; locust bean gum; polyurethane; polyether polyurethane; cellulose; cellulose ethers (for example methylcellulose, carboxymethyl cellulose, ethylcellulose, hydroxyethyl cellulose or hydroxypropyl cellulose), cellulose esters, cellulose acetates, cellulose triacetates; cross-bonded polyvinyl alcohol; polymers and copolymers of acrylic acid, hydroxyalkyl acrylates, hydroxyethyl acrylate, diethylene glycol monoacrylate, 2-hydroxypropylacrylate or 3-hydroxypropyl acrylate; carbomers (cross-linked poly(acrylic acids), for example carbomer 910, 934P, 940GE, 941GE, 971P, 974P; polymers and copolymers of methacrylic acid, hydroxyethyl methacrylate, diethyleneglycol monomethacrylate, 2-hydroxypropyl methacrylate, 3-hydroxypropyl methacrylate or dipropylene glycol monomethylacrylate; vinylpyrrolidone polymers; polymers and copolymers or acrylamide, N-methylacrylamide, N-propylacrylamide; methacrylamide, N-isopropylmethacrylamide, or N-2-hydroxyethylmethacrylamide; poloxamers (triblock copolymers comprising a central polyoxypropylene block flanked by two polyoxyethylene blocks, for example a Pluronic®); and gels comprising cross-linked polyalkylene glycols, for example gels comprising cross-linked polyethylene glycol or cross-linked polypropylene glycol. In specific embodiments binary or tertiary etc combinations of any of the above gel-forming agents are foreseen. When the gel forming agent comprises a PEG, the PEG is suitably a higher molecular weight than the PEG used as a solvent to dissolve or disperse the halogenated salicylanilide selected from niclosamide and oxyclozanide (particularly niclosamide), or a pharmaceutically acceptable salt or hydrate thereof, in the gel composition. Accordingly, it is to be understood that when the gel-forming agent is a PEG, the PEG of the gel-forming agent is different to the PEG present in component (ii) of the compositions of the invention. For example, where the gel forming agent comprises a PEG, the PEG suitably has a molecular weight greater than 600, for example greater than 1000, greater than 10000 or greater than 20000. Suitably, when the gel forming agent comprises a PEG it has an average molecular weight of from about 600 to about 35,000, for example from about 800 to about 25,000, or from about 1000 to about 20,000. Other gel-forming agents are also contemplated, for example as disclosed in Gels handbook Vols 1-4, Osada et al. 2001 Elsevier.

The gel-forming polymer may be a gum, for example a gum selected from tragacanth gum, xanthan gum; gum arabic (acacia gum); guar gum; gellan gum locust bean gum.

The gel-forming polymer may be a cellulose ether, for example methylcellulose, carboxymethyl cellulose, ethylcellulose, hydroxyethyl cellulose, hydroxy propyl methyl cellulose or hydroxypropyl cellulose.

### Carbomer Gel-Forming Polymers

In a particular embodiment the gel-forming agent is a carbomer. Carbomers are high molecular weight cross-linked poly(acrylic acid) polymers. The polymers may be cross-linked by polyalcohol allyl ethers, for example, allyl sucrose or allyl pentaerythritol The carbomer may be a homopolymer, for example 910, 934P, 940GE, 941GE, 971P, 974P, wherein “GE” refers to medical grade and “P” oral grade. Derivatives of Carbomer polymers may also be used, for example Carbopol interpolymers comprising a carbomer polymer comprising a block copolymer of polyethylene glycol and a long chain alkyl acid ester, such derivatives are commercially available as ETD 2020 NF and Ultrez 10 NF from Lubrizol.

Carbomers (also known as Carbopols) are well known and are characterised in the United States Pharmacopeia/National Formulary (USP/NF) monograph for Carbomers and the European Pharmacopeia (Ph. Eur.) monograph for Carbomers, reference to which is incorporated herein.

The carbomer may have a viscosity of from about 4,000 to about 70,000, for example about 10,000 to about 60,000, for about 20,000 to about 50,000, about 25,000 to about 45,000 or about 29,400 to about 39,400 cP, wherein the viscosity is that of a 0.5 wt % solution of the carbomer in water, neutralised to pH 7.3-7.8 at 25° C., measured using a Brookfield RVT, 20 rpm, spindle #6.

Suitably the carbomer comprises from about 56% to about 68.0% by weight carboxylic acid (—COOH) groups. The proportion of carboxy groups present in the carbomer may be determined using known methods, for example by titrating an aqueous solution or dispersion of the polymer against NaOH.

Suitably the carbomer is substantially free of residual benzene (for example containing less than 0.5 parts per million). Accordingly, it is preferred that the carbomer is prepared without using benzene as a solvent during the polymerisation process. Preferred carbomers are those are prepared using ethyl acetate and optionally cyclohexane as the solvent during polymerisation.

A particular carbomer for use as a gelling agent in the present invention is Carbomer 974P. This carbomer suitably has a viscosity of 29400 to 39400 cP (0.5% solution in water neutralized to pH 7.3-7.8 and measured at 25° C. using a Brookfield RVT, 20 rpm with spindle #6). The carbomer typically has a carboxylic acid content of from 56 to 68%.

Conventionally carbomer gels are formed by dispersing the carbomer in water, which results in ionisation of the carboxy groups present in the polymer. The resulting solution or dispersion is then neutralised using a base, resulting in an increase in viscosity and gel formation. However, in the present invention the gel is a non-aqueous gel and gel formation may be achieved by dissolving or dispersing the carbopol in the organic solvent together with the halogenated salicylanilide selected from niclosamide and oxyclozanide (particularly niclosamide), or a pharmaceutically acceptable salt or hydrate thereof, and heating the mixture to about 70° C.

The gel-forming polymer may also be referred to as a colloid i.e. a colloid system wherein the colloid particles are dispersed in the organic solvent and the quantity of solvent available allows for the formation of a gel. In embodiments it is preferred to use reversible colloids preferably thermo-reversible colloids (e.g. agar, agarose and gelatin etc.) as opposed to irreversible (single-state) colloids. Thermo-reversible colloids can exist in a gel and sol state, and alternate between states with the addition or elimination of heat. Thermoreversible colloids which may be used according to the invention, whether individually or in combination, include for example, gelatin, carrageenan, gelatin, agar, agarose (a polysaccharide obtained from agar), pectin and cellulose derivatives for example methylcellulose, carboxymethyl cellulose, ethylcellulose, hydroxyethyl cellulose, hydroxy propyl methyl cellulose or hydroxypropyl cellulose. Another term which may be applied to gel forming polymers is “thermotropic”: a thermotropic gelling agent is one caused to gel by a change in temperature. In embodiments of the invention, therefore, the gel former is a thermotropic gel-forming polymer or a combination of such polymers.

The gel-forming polymer may be or comprise an ionotropic gel-forming polymer whose gelling is induced by ions. Suitable ionotrophic gel-forming agents are anionic or cationic polymers which can be cross-linked by multivalent counter ions to form a gel. The ionotropic gel-forming polymers may be, for example chitosan, an alginate, carrageenan or pectin.

The gel-forming polymer may comprise or be a single gel-forming polymer or a mixture of two or more gel-forming polymers. For example, the gel-forming polymer may comprise a combination of two or more of the gel-forming polymers listed herein.

The amount of gel forming agent present in the composition should be selected so as to provide a gel composition having the required rheological properties, for example a viscosity suitable for topical application. Generally, the gel composition will be of a viscosity such that it can be readily dispensed and spread over and rubbed in the area of, for example, skin that is infected. The rheology of the gel composition will depend upon the particular gelling agent used, the molecular weight of the PEG, and the amount of the halogenated salicylanilide selected from niclosamide and oxyclozanide (particularly niclosamide), or a pharmaceutically acceptable salt or hydrate thereof, in the composition. Generally, the gelling agent, for example a carbomer, will be present in the gel composition is an amount of up to about 10% by weight, for example up to about 1%, 2%, 3%, 4%, 5%, 5.5%, 6%, 6.5%, 7%, 7.5%, 8%, 8.5%. 9% or 9.5% by weight of the gel composition. Suitably the gelling agent, for example a carbomer, may be present in an amount of from about 0.01% to about 10% by weight of the gel composition, for example about 0.01% to about 8%, about 0.05% to about 7%, about 0.05% to about 6%, about 0.05% to about 5%, about 0.05% to about 4%, about 1% to about 6%, about 1% to about 5% or about 1% to about 4%, about 2% to about 5%, about 2% to about 4% or about 2% to about 3%, wherein the % is by weight based on the weight of the gel composition.

### Polyethylene Glycol (PEG)

In embodiments where PEG is present in the compositions comprising the halogenated salicylanilide selected from niclosamide and oxyclozanide (particularly niclosamide), or a pharmaceutically acceptable salt or hydrate thereof, as described herein, the PEG suitably has one or more of the characteristics described in this section.

Suitably the PEG is liquid at ambient temperature (for example 20 to 25° C.), accordingly the solvent may be a low molecular weight PEG. Particularly, the PEG has an average molecular weight of 600 or less, suitably less than about 600. For example, the PEG may have an average molecular weight of from about 200 to about 600, about 200 to about 500 or about 200 to about 400. A particular PEG is selected from PEG 200, PEG 300 and PEG 400. In one particular embodiment the PEG is PEG 400. Alternatively, the PEG may comprise a mixture of PEGs which together with the other components of the composition provide a composition which is suitable for e.g. topical application to the subject. Accordingly, the PEG may be a mixture of one or more low molecular weight PEGs with one or more higher molecular weight PEG, wherein the mixture of PEGs has a melting point below 40, or preferably below about 37° C.

Suitably the PEG is present in an amount at least sufficient to provide a solution of the halogenated salicylanilide selected from niclosamide and oxyclozanide (particularly niclosamide) in the composition. As will be realised the amount of PEG required to dissolve the halogenated salicylanilide selected from niclosamide and oxyclozanide (particularly niclosamide) will depend also on the other components of the composition. In certain embodiments the PEG is present in the composition of the invention an amount of at least 60%, suitably greater than 60% by weight of the composition. Non-aqueous compositions containing high amounts of PEG provide topical compositions which give high levels of the halogenated salicylanilide selected from niclosamide and oxyclozanide (particularly niclosamide) in skin tissues and only minimal systemic exposure to the halogenated salicylanilide selected from niclosamide and oxyclozanide (particularly niclosamide). Such compositions have also been found to be well tolerated, despite containing high PEG concentrations. Suitably the PEG is present in an amount of greater than 60%, 65%, 70%, 75%, 80%, 85%, 90%, 91%, 92%, 93%, 94%, 95%, 96%, 97% 98% or 99% wherein the % is by weight based upon the weight of the composition. It may be that the PEG, preferably a PEG with an average molecular weight of 600 or less (particularly less than 600) is present in the non-aqueous composition of the invention in an amount of for example 65 to 98%, for example from 65% to 95%, 65% to 90%, 65% to 80%, 70% to 98%, 70% to 95%, 70% to 85%, 70% to 80%, 80% to 98%, 80% to 95%, 80% to 90%, 85% to 98% or 85% to 95%, wherein the % is by weight based upon the weight of the non-aqueous composition of the invention.

In certain embodiments the composition (e.g. a non-aqueous composition) comprise lower concentrations of PEG, for example 50% or less, 45% or less, 40% or less, 35% or less 30% or less, 25% or less, 20% or less, 15% or less, wherein the % is % by weight of the composition. It may be that the PEG is present from about 1% to about 50%, from about 5% to about 40%, from about 5% to about 35%, or from about 5 to about 30% by weight of the composition.

### Topical Foam Compositions

In certain embodiments the halogenated salicylanilide selected from niclosamide and oxyclozanide (particularly niclosamide), or a pharmaceutically acceptable salt or hydrate thereof, is formulated as a foam composition. The foam composition may be an aqueous foam composition such as an emulsion or nano-emulsion foams or a water-alcohol based foam (e.g. a water-ethanolic foam). Alternatively, the foam may be a non-aqueous (i.e. water-free) foam composition, including but not limited to oil-based foams, petrolatum-based foams, ointment foams; emollient foams and foams formed using non-aqueous hydrophilic excipients. When the foam is a foam formed from an emulsion, the emulsion may be a water-in-oil emulsion or an oil-in-water emulsion comprising the halogenated salicylanilide selected from niclosamide and oxyclozanide (particularly niclosamide), or a pharmaceutically acceptable salt or hydrate thereof. Foams suitable for the delivery of pharmaceuticals are well-known and are described in for example Arzhavitina et al, “Foams for pharmaceutical and cosmetic application” Int. J. Pharm., 394, 1-17 (2010).

Suitably the foam is a breakable foam, i.e. a thermally stable foam which collapses (breaks) upon application of shear stress to the foam. Such breakable foams can be applied to the skin as a foam and then collapse when the foam is rubbed into the skin, thereby enabling the active to be applied to the skin in the area required.

In certain embodiments the foam is an emollient foam formed from an oil-in-water emulsion comprising the halogenated salicylanilide selected from niclosamide and oxyclozanide (particularly niclosamide), or a pharmaceutically acceptable salt or hydrate thereof. The oil may be, for example. a mineral oil, a plant derived oil (e.g. olive oil, soybean oil, coconut oil, or castor oil), medium or long-chain triglycerides and esters thereof, fatty acids, fatty acid esters, fatty acid alcohols and a wax. For example, the oil may comprise an alcohol selected from lauryl alcohol, myristyl alcohol, cetyl alcohol, stearyl alcohol, arachidyl alcohol, behenyl alcohol, tetracosanol, hexacosanol, octacosanol, triacontanol, and tetratriacontanol. The oil may comprise a fatty acid selected from dodecanoic acid, tetradecanoic acid, hexadecanoic acid, heptadecanoic acid, octadecanoic acid, eicosanoic acid, docosanoic acid, tetracosanoic acid, hexacosanoic acid, heptacosanoic acid, octacosanoic acid, triacontanoic acid, dotriacontanoic acid, tritriacontanoic acid, tetratriacontanoic acid and pentatriacontanoic acid. The oil may comprise a hydroxy fatty acid such a 12-hydroxy stearic acid. The oil may comprise a wax, for example carnauba wax, candelilla wax, ouricury wax, sugarcane wax, retamo wax, jojoba oil, an animal wax (e.g. beeswax) or a petroleum derived wax (e.g. paraffin wax).

The emulsion may include emulsifiers or surfactants to stabilise the emulsion, for example one or more non-ionic surfactant (including any of the surfactants described herein, particularly those in relation to the non-aqueous topical compositions described above). The foam may comprise further excipients, for example, solvents, gelling agents, humectants, preservatives, and absorption enhancers, including but not limited to those described herein.

In a particular embodiment the foam is a non-aqueous foam. Such foams can be prepared by forming one of the non-aqueous formulations described above, for example a non-aqueous gel composition, into a foam composition. Examples of non-aqueous foam compositions which may be suitable for the delivery of a halogenated salicylanilide such as the halogenated salicylanilide selected from niclosamide and oxyclozanide (particularly niclosamide) are described in, for example WO2010/041141, WO2009/098595 and WO2008/152444.

In certain embodiments the foam is a non-aqueous oil-based foam prepared using a suitable pharmaceutically acceptable oil, for example as discussed above in relation to emollient foams in which the halogenated salicylanilide selected from niclosamide and oxyclozanide (particularly niclosamide), or a pharmaceutically acceptable salt or hydrate thereof, is dispersed or dissolved. It may be that surfactants are used to stabilise the foams. It is also contemplated that non-aqueous oil-based foams may be prepared which do not require a surfactant. Such foams include but are not limited to those described in WO2011/013008, WO2011/013009, WO2011/064631 and WO2011/039637.

Other examples of foam compositions that may be used to formulate the halogenated salicylanilide selected from niclosamide and oxyclozanide (particularly niclosamide), or a pharmaceutically acceptable salt or hydrate thereof, include analogous compositions to those described in, for example, WO2011/138678, WO2011/039638, WO/2010/125470, WO/2009/090558, WO2009/090495, WO2009/007785, WO2008/038140, WO2007/085902, WO2007/054818, WO2007/039825, WO2006/003481, WO2005/018530, WO2005/011567 and WO2004/037225.

Foam compositions comprising the halogenated salicylanilide selected from niclosamide and oxyclozanide (particularly niclosamide), or a pharmaceutically acceptable salt or hydrate thereof, are suitably formulated as a semi-solid or liquid composition packaged in a suitable aerosol pressurised container with a propellant. The foam is formed upon release of the composition from the pressurised container via a suitable aerosol nozzle in the outlet of the container. Suitable propellants include a hydrocarbon propellant such as propane or butane, or a halogenated fluorocarbon such as tetrafluoroethane. Suitable aerosol containers and nozzles are well-known.

### Optional Components for Topical Compositions

The following components and features may optionally be present in the the halogenated salicylanilide selected from niclosamide and oxyclozanide (particularly niclosamide) compositions described herein, for example the non-aqueous topical compositions described herein.

### Solvents

The topical composition may comprise one or more solvent(s). The presence of a further solvent may enhance the solubility of the halogenated salicylanilide selected from niclosamide and oxyclozanide (particularly niclosamide) and or help maintain niclosamide in solution during the preparation, storage and topical use of the non-aqueous composition. The additional solvent may be, for example, a polar organic solvent in which the halogenated salicylanilide selected from niclosamide and oxyclozanide (particularly niclosamide) is soluble, for example a polar organic solvent wherein the halogenated salicylanilide selected from niclosamide and oxyclozanide (particularly niclosamide) has a solubility of greater than 2% by weight in the additional solvent.

The polar organic solvent may be a protic polar organic solvent. In one embodiment the solvent is a protic polar organic solvent having a dielectric constant of from about 10 to about 45, for example a dielectric constant of from about 10 to about 25. Particular polar protic organic solvents are those which have a dielectric constant of from about 10 to about 20, wherein in each case the dielectric constant is measured at 20-25° C. The dielectric constant of organic solvents is well known or can be measured using well-known techniques

Representative protic polar organic solvents with a dielectric constant in the range of 10 to 45 include those set out in Table 1:

TABLE 1

| Solvent | Dielectric Constant at 20-25° C. |
|---|---|
| 2-methylpentane-2,4-diol (pinakon) | 7.4 |
| PEG 300 | 18.0 |
| PEG 400 | 14.1-12.4 |
| PEG 600 | 12.7 |
| N-octanol | 10.3 |
| Propylene glycol | 32 |
| Glycerol | 42.5 |
| Methanol | 33 |
| Ethanol | 24.3 |
| Propanol | 22 |

Further polar organic solvents with a dielectric constant in the range are well known (see for example “Solubility and Solubilization in Aqueous Media” By Samuel H. Yalkowsky (University of Arizona). Oxford University Press: New York. 1999). For example, the polar organic solvent may be selected from ethyl acetate, dimethylformamide, dichloromethane, glycerol, propylene glycol, or 2-(2-ethoxyethoxy)ethanol (Transcutol), propylene glycol stearyl ether and propylene glycol isostearate.

In embodiments the polar organic solvent is an aprotic polar organic solvent having a dielectric constant of from about 10 to about 45, for example a dielectric constant of from about 10 to about 25 at 25° C.

When present the additional solvent(s) is suitably present in an amount of up to 35% by weight of the composition. For example, up to 30%, 25%, 20% 15% or 10% by weight of the composition. In particular embodiments the additional solvent(s) is present in an amount of less than 10%, for example less than 8%, less than 6%, less than 5% or less than 3%, wherein the % is by weight based upon the weight of the non-aqueous composition. It may be that the additional solvent is present in an amount of 1% to 30%, from 1% to 25%, from 1% to 20%, from 1 to 10%, from 3 to 30%, from 3 to 20%, from 3 to 15%, from 5 to 30%, from, 5 to 20% or from 5 to 10%, wherein the % is by weight based upon the weight of the composition.

### Non-Ethanolic Compositions

The presence of ethanol in topical compositions can cause dryness and/or peeling of the skin, particularly in patients with sensitive skin. This can be a particular problem in patients with dermal conditions such as dermatitis (e.g. AD). Accordingly, in certain embodiments the topical composition comprising the halogenated salicylanilide selected from niclosamide and oxyclozanide (particularly niclosamide), or a pharmaceutically acceptable salt or hydrate thereof, is ethanol free. Thus, in a preferred embodiment the topical halogenated salicylanilide composition comprises a non-aqueous, non-ethanol (ethanol free) composition, for example a non-aqueous, non-ethanol gel composition.

### Absorption Enhancers

The topical composition may optionally comprise an absorption enhancer. The absorption may be any substance which acts to enhance the permeation of the halogenated salicylanilide selected from niclosamide and oxyclozanide (particularly niclosamide) into the epidermis and epidermis. Suitable absorption enhancers include the transdermal absorption enhancers disclosed in for example Smith and Maibach (2005) Percutaneous Penetration Enhancers, Second Edition ISBN 9780849321528, incorporated herein by reference.

It may be that the absorption enhancer, when present in the topical composition is selected from, for example, a sulfoxide (for example dimethylsulfoxide); dimethylacetamide; dimethylformamide; a urea; a fatty alcohol, for example a C₈-C₁₈ fatty alcohol, which may be saturated or unsaturated (for example caprylic alcohol or cetostearyl alcohol); a polyol (for example glycerol; a glycol (for example propylene glycol or hexylene glycol); Azone ((1-dodecylazacycloheptan-2-one); an essential oil (for example a terpene or terpenoid); a pyrrolidone (for example N-methyl-2-pyrrolidone); an oxazolidinone (for example 4-decyloxazolidin-2-one) a surfactant (for example a non-ionic, anionic or cationic surfactant, particularly a non-ionic surfactant for example a polyoxyethylene glycol sorbitan alkyl ester (for example polysorbates such as Polysorbate 80 ((polyoxyethylene (20) sorbitan monooleate), Polysorbate 60 (polyoxyethylene (20) sorbitan monostearate), Polysorbate 40 (polyoxyethylene (20) sorbitan monopalmitate) or Polysorbate 20 (polyoxyethylene (20) sorbitan monolaurate)), a polyoxyethylene glycol alkyl ether (Brij surfactants e.g. polyethoxylated stearyl ethers such as Brij S721 (a polyoxyethylene fatty ether derived from stearyl alcohols) or Brij S2 (Polyoxyethylene (2) stearyl ether)), a poloxamer or a PEGylated fatty acid glyceride such as caprylocaproyl polyoxyl-8 glycerides (e.g. Labrasol), a fatty acid ester of glycerol, for example glyceryl stearate, or polyoxyethylene ethers of fatty alcohols (for example cetyl alcohol and/or stearyl alcohol, particular examples include ceteareth-15, -16, -17, -18, -19, -20, -21, -22, 23-, -24, or -25 and particularly ceteareth-20), a polyethoxylated sorbitan fatty acid ester, for example. The absorption enhancer may also be 2-(2-ethoxyethoxy)ethanol (Transcutol). Preferred absorption enhancers are those which have a minimal impact on the structure of the skin so as to minimise undesirable tolerability effects associated with the absorption enhancer, for example irritation, which could exacerbate the dermatitis (e.g. AD) in the subject. Particular absorption enhancers include polyols, for example propylene glycol or glycerol. Accordingly the absorption enhancer may be propylene glycol. The absorption enhancer may be glycerol. It is to be understood that where the absorption enhancer may also act as an additional solvent in the composition, particularly when the halogenated salicylanilide selected from niclosamide and oxyclozanide (particularly niclosamide) is soluble in the absorption enhancer.

When present the absorption enhancer may be in an amount of up to 35% by weight of the topical composition (e.g. a gel composition), for example from 0.5% to 35%, from 1% to 35%, from 5% to 30%, from 10% to 30%, from 5% to 35%, from 5% to 30% or from 10% to 30%, wherein the % is by weight of the composition.

### Other Ingredients

The halogenated salicylanilide compositions described herein (e.g. a topical composition) may comprise one or more additional excipients in addition to the halogenated salicylanilide selected from niclosamide and oxyclozanide (particularly niclosamide), or a pharmaceutically acceptable salt or hydrate thereof, and the other excipients described above (e.g. PEG in a non-aqueous topical composition). Additional excipients may be selected to provide compositions of the required form for topical administration. The additional excipients may be, for example one or more excipients selected from viscosity modifying agents, emulsifiers, surfactants, humectants, oils, waxes, solvents, preservatives, pH modifying agents (for example a suitable acid or base, for example an organic acid or organic amine base), buffers, antioxidants (for example butylated hydroxyanisol or butylated hydroxytoluene), crystallisation inhibitors (for example a cellulose derivative such as hydroxypropylmethyl cellulose), colorants, fragrances. Representative examples of such additional excipients are well known, for example as listed in the Handbook of Pharmaceutical Excipients, r Edition, Rowe et al. Furthermore specific excipients are set out in any of the non-aqueous compositions described in the Examples herein.

### Manufacture of Topical Compositions

The topical compositions described herein may be manufactured using well-known methods. For example, the non-aqueous gel compositions comprising PEG may be prepared by a process comprising the steps:

(i) dissolving the halogenated salicylanilide selected from niclosamide and oxyclozanide (particularly niclosamide), or a pharmaceutically acceptable salt or hydrate thereof, in the PEG;
(ii) combining the solution from step (i) with the gel-forming agent to form a mixture; and
(iii) causing the mixture to gel.

Suitably the halogenated salicylanilide selected from niclosamide and oxyclozanide (particularly niclosamide) is completely dissolved in the PEG in step (i) to form a solution. Dissolution may be aided by agitation of the mixture by stirring or by the application ultrasound. Optionally the mixture may be heated to facilitate dissolution. However, preferably the solution is prepared at ambient temperature. Optionally any halogenated salicylanilide selected from niclosamide and oxyclozanide (particularly niclosamide) that remains undissolved may be removed by a suitable filtration or other separation method prior to combining the solution with the gel-forming agent in step (ii) of the process.

The solution from step (i) may be added to the gel-forming agent or, alternatively, the gel-forming agent may be added to the solution. Optionally the gel-forming agent may be dissolved in some of the PEG to form a solution or dispersion prior to combining it with the solution from step (i). Suitably any additional optional components of the gel-composition, such as absorption enhancers, additional solvents etc. are added to the mixture prior to gelation of the composition. Alternatively, one or more of the optional components can be added after gel formation by mixing the additional component(s) with the gel.

Gel formation in step (iii) may be affected by various methods, depending on the nature of the gel-forming agent used. For example, where the gel-forming agent is thermotropic, the gel forming agent may be heated to form a liquid prior to adding the solution from step (i). Following mixing of the gel-forming agent with the solution, the resulting mixture may be cooled thereby causing the mixture to gel. Alternatively, where gelling is effected by ionic cross-linking, a suitable ionic agent is added to the mixture in step (iii), for example a suitable salt to thereby cause the mixture to gel. Gelling may also be induced by changing the pH of the mixture using a suitable acid or base to achieve the required pH for gelling to occur. The process is suitably carried out using anhydrous reagents under anhydrous conditions to ensure that the resulting gel composition is a non-aqueous gel composition.

When the gel-forming agent is a carbomer, a particular process for the preparation of the non-aqueous gel composition comprises:

(i) dissolving the halogenated salicylanilide selected from niclosamide and oxyclozanide (particularly niclosamide), or a pharmaceutically acceptable salt or hydrate thereof, in the PEG;
(ii) combining the solution from step (i) with a carbomer to form a mixture; and
(iii) heating the mixture to form a gel.

Step (i) of this process is suitably performed at room temperature. After combining the solution with the carbomer the mixture is mixed to provide a uniform dispersion. Mixing can be performed using any suitable method, for example stirring or, preferably, by homogenisation. The resulting dispersion is suitably de-gassed prior to gel formation in step (iii).

In step (iii) the mixture is suitably heated to a temperature of 60 to 80° C., for example at about 70° C., preferably under agitation. The mixture may be held at this temperature for a sufficient time to form a homogenous and transparent dispersion and to effect gel formation. Typically a holding time of about 30 minutes is sufficient to enable solvation of the carbomer and gel formation.

The process is suitably performed under anhydrous conditions using anhydrous reagents to ensure that the resulting gel composition is a non-aqueous gel.

When the composition of the invention is in the form of a lotion, ointment or cream the composition may be prepared using known methods for the preparation of such compositions. For example, lotion or ointments may be prepared by simply blending the halogenated salicylanilide selected from niclosamide and oxyclozanide (particularly niclosamide), or a pharmaceutically acceptable salt or hydrate thereof, and the other excipients comprising the formulation, for example viscosity modifiers, solvents and/or surfactants.

Non-aqueous topical compositions may also be prepared as non-aqueous emulsion or microemulsions to provide a composition in the form of, for example a non-aqueous cream. Non-aqueous emulsions and microemulsions may be prepared using well known methods. Non-aqueous emulsions and microemulsions may be prepared by mixing two immiscible non-aqueous phases. Suitably a non-aqueous hydrophilic phase (for example a hydrophilic phase comprising polar excipients and the halogenated salicylanilide selected from niclosamide and oxyclozanide (particularly niclosamide), or a pharmaceutically acceptable salt or hydrate thereof) is emulsified with an immiscible hydrophobic phase (e.g. comprising non-polar hydrophobic excipients). The non-aqueous emulsion may comprise a continuous hydrophobic phase and a discontinuous hydrophilic phase. Generally however, the non-aqueous emulsion will comprise a continuous hydrophilic phase and a discontinuous hydrophobic phase. It may be that the non-aqueous hydrophilic phase comprises the halogenated salicylanilide selected from niclosamide and oxyclozanide (particularly niclosamide) and PEG and the non-aqueous hydrophobic phase comprises a non-polar liquid, which is immiscible with the hydrophobic phase, for example a medium chain triglyceride, a vegetable oil, a hydrocarbon oil or a mineral oil such as a paraffin. Generally the non-aqueous emulsion will be stabilised by one or more suitable surfactants or emulsifiers, for example one or more non-ionic surfactants (e.g. macrogol cetostearyl, cetostearyl alcohol, glyceryl stearate, polysorbate 80, Brij s721, Brij S2, ceteareth-20 or macrogol stearyl ether). The emulsion or micro emulsion may be formed using well-known methods, for example by homogenisation of the hydrophilic phase with the hydrophobic phase together with the other components of the non-aqueous emulsion or microemulsion.

### Dosages and Dosage Regimens

An effective amount of the halogenated salicylanilide selected from niclosamide and oxyclozanide (particularly niclosamide), or a pharmaceutically acceptable salt or hydrate thereof, for use in the treatment of dermatitis (e.g. AD) is an amount sufficient to relieve the subject of one or more of the symptoms of dermatitis (e.g. AD) described herein or to slow the progression or development of dermatitis (e.g. AD).

The amount of active ingredient that is combined with one or more excipients to produce a single dosage form will necessarily vary depending upon the host treated and the particular route of administration. For example, a formulation intended for topical administration to subjects (e.g. humans) will generally be administered in an amount sufficient to cover the dermatitis lesion. Suitably the composition is applied in an amount to provide a dose of the halogenated salicylanilide selected from niclosamide and oxyclozanide (particularly niclosamide), or a pharmaceutically acceptable salt or hydrate thereof, of from about 0.001 to about 1 mg/cm²; about 0.01 to about 0.5 mg/cm²; about 0.01 to about 0.5 mg/cm²; or about 0.01 to about 0.3 mg/cm², for example about 0.04, 0.05, 0.06, 0.07, 0.08, 0.09, 1, 1.1, 1.2, 1.3, 1.4, 1.5, 1.6, 1.7, 1.8, 1.9, 2.0, 2.1, 2.2, 2.3, 2.4 or 2.5 mg/cm². The composition will be applied in an amount sufficient to provide this desired dose of the halogenated salicylanilide selected from niclosamide and oxyclozanide (particularly niclosamide). This will of course depend on the concentration of the halogenated salicylanilide selected from niclosamide and oxyclozanide (particularly niclosamide), or a pharmaceutically acceptable salt or hydrate thereof, in the composition. Typically, the composition will be applied in an amount of about 0.1 to about 50 mg/cm²; about 1 to about 20 mg/cm²; about 1 to about 5 mg/cm²; about 2 to 5 mg/cm²; about 2 to about 15 mg/cm² or about 4 to about 10 mg/cm².

When administered topically to the subject, the halogenated salicylanilide selected from niclosamide and oxyclozanide (particularly niclosamide), or a pharmaceutically acceptable salt or hydrate thereof, is suitably applied directly to a dermatosis lesion. Suitably the halogenated salicylanilide selected from niclosamide and oxyclozanide (particularly niclosamide), or a pharmaceutically acceptable salt or hydrate thereof, is topically applied in the form of a topical composition and is gently rubbed into the skin at the site of the lesion to be treated so as to provide coverage of substantially all of the lesion. Optionally a composition comprising the halogenated salicylanilide selected from niclosamide and oxyclozanide (particularly niclosamide), or a pharmaceutically acceptable salt or hydrate thereof, may be topically applied using a suitable carrier substrate, for example a wound dressing or a patch impregnated with or carrying a composition comprising the halogenated salicylanilide selected from niclosamide and oxyclozanide (particularly niclosamide), or a pharmaceutically acceptable salt or hydrate thereof. The carrier may be applied to a lesion such that the lesion is brought into contact with the halogenated salicylanilide selected from niclosamide and oxyclozanide (particularly niclosamide), or a pharmaceutically acceptable salt or hydrate thereof, present in or on the carrier substrate.

The frequency of (e.g. topical) administration of the halogenated salicylanilide selected from niclosamide and oxyclozanide (particularly niclosamide), or a pharmaceutically acceptable salt or hydrate thereof, depend upon a number of factors that may readily be determined by a physician, for example the severity of the dermatitis (e.g. AD). Suitably the halogenated salicylanilide selected from niclosamide and oxyclozanide (particularly niclosamide), or a pharmaceutically acceptable salt or hydrate thereof, is topically administered 1, 2, 3 or 4 times per day. The duration of the treatment may be, for example, 1 week or more, 2 weeks or more, 3 weeks or more, 4 weeks or more, 6 weeks or more, 8 weeks or more, 10 weeks or more, 12 weeks or more, 14 weeks or more, 16 weeks or more, 18 weeks or more, 20 weeks or more, 6 months or more, 7 months or more, 8 months or more, 9 months or more, 10 months or more, 11 months or more, or 1 year or more. The halogenated salicylanilide selected from niclosamide and oxyclozanide (particularly niclosamide), or a pharmaceutically acceptable salt or hydrate thereof, may, for example, be topically administered 1, 2 or 3 times per week.

The halogenated salicylanilide selected from niclosamide and oxyclozanide (particularly niclosamide), or a pharmaceutically acceptable salt or hydrate thereof, may be topically applied at least once daily, such as twice daily, for more than 5 days, such as more than 10 days, more than 15 days more than 20 days, more than 25 days or more than 30 days, to the skin of the subject.

### EXAMPLES

### Non-Aqueous Topical Niclosamide Gel Formulations

The topical gel compositions shown in Table 2 were prepared:

TABLE 2

| Composition | Formulation | Formulation |
|---|---|---|
| Raw material INCI or PhEur name | A | B |
| (trade name) | % (w/w) | % (w/w) |
| Niclosamide, anhydrous | 2.0 | 4.0 |
| Macrogol 400 | 95.6 | 93.6 |
| Carbomer 974P (Carbopol 974P) | 2.4 | 2.4 |

The composition was prepared as follows. Niclosamide 200 mg, PEG 400 (9.56 g for Formulation A and 9.36 g for Formulation B) were weighed in blue cap bottles. The mixture was stirred at room temperature until a clear solution formed. 240 mg. Carbomer 974P was then dispersed in the niclosamide PEG 400 solution. The dispersion was homogenized and degassed. The suspension was then heated at 70° C. and stirred mechanically at 250 rpm until a homogeneous dispersion formed after about 30 minutes. The final solution was then cooled to give the title non-aqueous gel compositions.

The final formulations were protected from light prior to further use.

The non-aqueous topical compositions shown in Tables 3 and 4 were also prepared.

TABLE 3

| | Formulation | Formulation | Formulation | Formulation |
|---|---|---|---|---|
| Composition | D | E | F | G |
| Appearance | Very shiny, | Shiny rather | Very shiny | Slightly shiny |
| | soft yellow | hard yellow | hard yellow | hard yellow |
| | ointment | ointment, | ointment, | ointment, |
| | | becomes soft | becomes soft | becomes soft |
| | | upon shearing | upon shearing | upon shearing |
| Raw material INCI | | | | |
| or PhEur name | | | | |
| (trade name) | % (w/w) | % (w/w) | % (w/w) | % (w/w) |
| Active | | | | |
| Niclosamide anhydrous | 2.0 | 2.0 | 2.0 | 2.0 |
| Hydrophilic Phase | | | | |
| Macrogol 400 | 30.0 | 20.0 | 27.5 | 30.0 |
| Propylene glycol | 25.0 | 15.0 | 17.5 | 15.0 |
| Ethoxydiglycol | | 15.0 | 5.0 | 15.0 |
| (Transcutol) | | | | |
| Glycerol | 19.7 | | | |
| Hydroxyethyl cellulose | 0.25 | | | |
| (Natrosol 250G Pharm) | | | | |
| Hydrophobic Phase | | | | |
| and emulsifiers | | | | |
| Polysorbate 80 | | 1.0 | 1.0 | 1.0 |
| Ceteareth-20 | 6.0 | | | |
| (Cetomacrogol 1000-PA) | | | | |
| Caprylocaproyl Macrogol | 10.0 | | | |
| 8 glycerides (Labrasol) | | | | |
| Steareth-21 (Brij S721) | | 5.0 | 5.0 | 5.0 |
| Steareth-2 (Brij S2) | | 5.0 | 5.0 | 5.0 |
| Macrogol Stearyl ether | | | 5.0 | |
| (Arlamol PS11E) | | | | |
| Paraffin, liquid | | 10.5 | 5.5 | 6.5 |
| Medium chain | | 6.0 | 6.0 | |
| triglycerides | | | | |
| Paraffin, Type 5205, hard | | 6.5 | 6.5 | 6.5 |
| Cetostearyl alcohol | 7.0 | 12.0 | 12.0 | 12.0 |
| (Kolliwax CSA 50) | | | | |
| Glyceryl stearate, Type II | | 2.0 | 2.0 | 2.0 |
| (Kolliwax GMS II) | | | | |

TABLE 4

| | Formulation | Formulation | Formulation |
|---|---|---|---|
| Composition | H | I | J |
| Appearance | Shiny rather | Very shiny | Soft shiny |
| | hard yellow | very soft | yellow |
| | ointment, | yellow | ointment, |
| | becomes | ointment, | becomes |
| | soft upon | becomes | softer |
| | shearing, | softer upon | upon |
| | liquefies at | shearing | shearing |
| | skin temp. | | |
| Raw material INCI | | | |
| or PhEur name | | | |
| (trade name) | % (w/w) | % (w/w) | % (w/w) |
| Active | | | |
| Niclosamide, anhydrous | 2.0 | 0.5 | 0.5 |
| Hydrophilic Phase | | | |
| Macrogol 400 | 10.0 | 20.0 | 10.0 |
| Propylene glycol | 20.0 | 10.0 | 20.0 |
| Ethoxydiglycol (Transcutol) | 15.0 | 15.0 | 15.0 |
| Hydrophobic Phase and | | | |
| emulsifiers | | | |
| Polysorbate 80 | 1.0 | 1.0 | 1.0 |
| Steareth-21 (Brij S721) | 5.0 | 5.0 | 5.0 |
| Steareth-2 (Brij S2) | 5.0 | 5.0 | 5.0 |
| Paraffin, liquid | 11.5 | 14.5 | 14.5 |
| Medium chain triglycerides | 10.0 | 10.0 | 10.0 |
| Paraffin, Type 5205, hard | 6.5 | 5.0 | 5.0 |
| Cetostearyl alcohol | 12.0 | 12.0 | 12.0 |
| (Kolliwax CSA 50) | | | |
| Glyceryl stearate, Type II | 2.0 | 2.0 | 2.0 |
| (Kolliwax GMS II) | | | |
| Carbomer 974P | | | |
| (Carbopol 974P) | | | |

The ointment formulations D, E, F, G, H, I and J set out in Tables 2 and 3 were prepared as non-aqueous emulsions using the following general method.

The hydrophilic phase of the emulsion and the anhydrous niclosamide (see under heading “hydrophilic phase” in Tables 2 and 3) were mixed together with stirring in a vessel to form a solution of the niclosamide in the hydrophilic phase. Generally the hydrophilic phase was heated gently at a temperature of about 60 to 75° C. (generally at about 70° C.) to aid dissolution of the niclosamide.

A hydrophobic phase comprising the oils and emulsifiers under the heading “Hydrophobic phase and emulsifiers” were mixed together by stirring in a heated vessel. The temperature was about 60 to 75° C. (generally at about 70° C.).

The hydrophobic phase and the hydrophilic phases were mixed together with gentle stirring so as to avoid phase separation and the mixture was cooled to a temperature of about 40 to 50° C. The mixture was then homogenised to give the final composition.

The appearance and some of the properties of the resulting compositions is described in the row marked “Appearance” in Tables 2 and 3.

### Example 1: Clinical Trial to Assess the Safety and Efficacy of Topically Applied Niclosamide in Healthy Volunteers and Patients with Atopic Dermatitis

### Study Design

A prospective, single centre, randomized, double-blind, Placebo controlled study in two Phases.

### Phase One of the Trial—Testing on Healthy Volunteers

### Primary Objective of Phase One of the Trial

The primary objective of the study is to demonstrate the safety and tolerability of topical niclosamide formulations in healthy volunteers.

### Secondary Objectives for Phase One of the Trial:

To determine the local and systemic exposure of the topical niclosamide composition.

### Exploratory Objective:

To collect illustrative information on local tolerability of the topical niclosamide composition.

To determine the best tolerated formulation to advance into Phase II of the trial.

### Patients in Phase One:

Randomization ratio 1:1; randomized niclosamide composition or Placebo application on right or left arm.

Inclusion criteria:
Signed and dated informed consent has been obtained.
Age 18-70 years.
Male or female.
Female subjects of childbearing potential must be confirmed not pregnant by a negative urine pregnancy test prior trial treatment.
Female subjects of childbearing potential must be willing to use effective contraceptive at trial entry until completion.
Male subjects must agree to use adequate contraception for the duration of the trial.

Exclusion criteria for Phase One of the study:
Regular use of medications unless considered clinically irrelevant by the Investigator.
Use of any dermatological drug therapy on the arms within 14 days before day 1 of this study.

### Phase One Protocol

Phase One of the study comprised a group with 30 healthy volunteers. Each of these volunteers were treated in four separate areas two times daily with the niclosamide topical formulations or the vehicle controls during a seven-day period.

The following topical niclosamide formulations were tested:
2% niclosamide non-aqueous dermal gel: Formulation A described in Table 1 above
2% niclosamide non-aqueous dermal cream: Formulation G described in Table 2 above

For each arm of the trial a placebo formulation comprising the vehicle only (i.e. without the niclosamide) was also tested.

### Dosage and Administration

Route of administration: topical.

Duration of treatment: 7 days.

Each volunteer had 4 formulations (2 active formulations and their respective Placebos) applied to defined skin areas in the dorsal arms. The body area to be treated was a circle marked by a skin marker with a diameter of 5 cm (approx. 20 cm²). The healthy volunteers had the body areas treated two times per day, at 08:00 (+/−2 hours) and 20:00 (+/−2 hours), respectively for 7 days. The expected dose of each formulation was 2 to 5 mg of product/cm²/day (corresponding to 0.04-0.1 mg niclosamide/cm²). The dermal formulation was left to dry for 10 minutes after application.

A screening visit was performed during Day −31 to −1. At Day 1 the patients were randomized, and this was also be the first day of treatment. On days 1-7 the healthy volunteers were treated twice daily at the study site. On Day 8 a final dose was applied in conjunction with the PK analysis. A final examination (end of study) was made on Day 15.

Six additional healthy volunteers were also enrolled for method testing. Treatments were blinded to both volunteers and doctors. The body area to be treated was be a circle with a diameter of approximately 5 cm and the expected dose of 2-5 mg of product/cm² (0.04-0.1 mg active substance per cm²).

The healthy volunteers in the trial were also subjected to a PK analysis after the last dose. The PK analysis involved sampling of blood after the final exposure to assess systemic exposure to niclosamide and skin biopsy sampling to assess local exposure to niclosamide in the skin. The 30 healthy volunteers were randomized for single punch biopsies to collect 10 biopsy samples from each active formulation. This meant that 1 active treatment area for each healthy volunteer had to be unblinded prior to biopsy sampling. To ensure that this did not interfere with the blind assessment of the safety of the formulations, safety was assessed in the morning of day 8, then on day 8 a 15th dose was given in conjunction with the bioanalysis. Biopsies were taken 1h (+/−10 min) after application of the respective formulation.

### Punch Biopsies

The skin biopsies were taken using sterile single use disposable biopsy punches (BP40F, Kai Europe GmbH, Solingen, Germany). For the 6 non-treated healthy volunteers, biopsies were taken on Day 1. 10 mL of blood was collected at day 1 for the method validation group to determine niclosamide concentration in the blood.

For the 30 treated healthy volunteers skin biopsies were taken on day 8 one hour after the 15^{th} application (+/−10 minutes).

The concentration of niclosamide in the skin biopsy samples was determined using validated bioanalytical UPLC-MS/MS methods.

The following chromatographic conditions were used:

| Parameter | Value | |
|---|---|---|
| Column | ZIC-cHILIC, 3 μm, 100 Å, | |
| | PEEK 100 × 2.1 mm, HX56413157 | |
| Column Temperature | 40° | |
| Mobile phase | A: 20 mM ammonium formate (pH = 3.5) | |
| | B: CH₃CN | |
| Mobile Phase Flow Rate | 0.4 ml/min, gradient mode | |
| | | Mobile |
| Gradient Program | Time (min) | phase B % |
| | 0.0 | 50 |
| | 1.0 | 50 |
| | 2.0 | 70 |
| | 2.2 | 50 |
| | 3.5 | 50 |

| | |
|---|---|
| Injection volume | 1 μl |
| Autosampler Temperature | 4° |
| Time of analysis | 3.5 min |
| Detection | MRM |

Mass spectrometry was performed using a Shimadzu 8050 mass spectrometer operating in Electrospray negative mode (ESI-ve).

### Preparation of Skin Biopsy Samples Containing Niclosamide

Extraction of skin biopsy samples was performed as follows:

1. Cut the tissue into small pieces and extract with 5.0 ml of DMSO/acetonitrile (50/50 v/v) at room temperature overnight using a shaker.
2. Spin down the tissue at 3700 g, collect supernatant and store it in a freezer (−20° C.).

### Determination of Niclosamide Concentration in Skin Biopsies

50 μl of untreated human skin extract was spiked with 10 μl of working standard solution (concentration of standard will be provided for each parameter). Samples were vortexed, then 200 μl of methanol/water solution 1;1 (v:v) was added. Finally, samples were centrifuged for 10 minutes at 4° C. at 2000 g. The supernatant was transferred into HPLC plate and analysed using UPLC-MS/MS.

### Assessment of Local Tolerability

Local dermal tolerability at the sites of application of the topical formulations was assessed by the investigator at all treatment visits using an 8-point dermal assessment score, in accordance with the FDA guideline on Skin Irritation and Sensitization Testing (1999). A dermal assessment score of 0 to 7 was defined as follows:
0=no evidence of irritation,
1=minimal erythema, barely perceptible,
2=definite erythema, readily visible; minimal edema or minimal papular response,
3=erythema and papule,
4=definite edema,
5=erythema, edema, and papule,
6=vesicular eruption,
7=strong reaction spreading beyond test site.
   Results from Phase I of the Trial

All of the topical dermal niclosamide formulations and placebo formulations were well tolerated with no signs of adverse reactions at the sites of administration. All 6 investigational medicinal products were scored at 0 in all subjects at all time points, see Table 5.

TABLE 5
Mean Local Tolerability Scores at Treatment Visits

| | Mean Local Tolerance Score | | | | | | |
|---|---|---|---|---|---|---|---|
| Formulation | Day 2 | Day 3 | Day 4 | Day 5 | Day 6 | Day 7 | Day 8 |
| A: niclosamide GEL 2% | 0.0 | 0.0 | 0.0 | 0.0 | 0.0 | 0.0 | 0.0 |
| A: niclosamide GEL Placebo | 0.0 | 0.0 | 0.0 | 0.0 | 0.0 | 0.0 | 0.0 |
| G: niclosamide CREAM 2% | 0.0 | 0.0 | 0.0 | 0.0 | 0.0 | 0.0 | 0.0 |
| G: niclosamide CREAM Placebo | 0.0 | 0.0 | 0.0 | 0.0 | 0.0 | 0.0 | 0.0 |

Systemic exposure to niclosamide was minimal with mean serum concentration of niclosamide 0.24 ng/mL, while local exposure to the skin was substantial (see Table 6).

TABLE 6
Niclosamide Concentration in Skin Biopsies (Phase 1 of Study)

| | | Anhydrous CREAM |
|---|---|---|
| | GEL 2% | 2% |
| | Formulation A | Formulation G |
| μg/g niclosamide | 5.8 ± 4.3 | 7.6 ± 4.9 |

Results from Phase One of the study showed that the dermal niclosamide and placebo formulations tested were well tolerated locally with no signs of adverse reactions at the sites of administration. No safety concerns were identified, and formulations delivered therapeutically relevant concentrations to the skin with minimal systemic exposure.

### Example 2: A Double-Blind, Randomized, Intraindividual Vehicle-Controlled, Phase II Study to Evaluate Efficacy and Safety of Topically Applied Niclosamide in Patients with Moderate Atopic Dermatitis

The topical niclosamide Formulation G, as described in Table 2 above, comprising 2% niclosamide was tested in the following clinical trial.

### Study Design

31 patients with moderate atopic dermatitis (Investigator Global Assessment [IGA] of 3) were included in this double-blind, randomized, intraindividual vehicle-controlled, Phase 2 study to evaluate the efficacy and safety of topically applied niclosamide. Patients had at least 2 areas of at least 3×3-cm of atopic dermatitis with a Total Sign Score (TSS) of ≥2 patients discontinued the study before Day 22.

Patients received topical applications of the niclosamide 2% composition and vehicle once daily for 3 weeks, followed by a 1-week follow up period. Topical niclosamide 2% and vehicle was applied on two separate target lesions of atopic dermatitis (lesions of at least 3×3-cm that are at least 2 cm apart, excluding the face, scalp, genitals, hands, and feet). The application areas (5×5-cm) were randomized (1:1) to once daily application of niclosamide 2% or vehicle at 5 mg/cm²/day without occlusion 6 days per week. Patients came to the study site for all study product application for a total of 3 weeks.

Efficacy was assessed using Total Sign Score (TSS) and Treatment Areas Assessment (TAA). Safety was assessed with vital signs, physical examination, clinical laboratory tests (haematology; biochemistry; urinalysis), and by collecting adverse events (AEs).

Three skin biopsies were collected in all patients (one from lesional skin at baseline, pre-dosing at Day 1, and two from lesional skin at Day 22 (one where topical niclosamide 2% had been applied and one where the vehicle had been applied). The lesional skin biopsies were analysed for skin thickness and inflammation biomarkers.

### Study Endpoints

Primary Endpoint:
Number of local and systemic treatment-emergent adverse events (AEs) in each treatment group (during 34 days).

Secondary Endpoints:
Change from baseline (pre-dosing at Day 1) in lesional TSS at Days 8, 15 and 22.
Change from baseline (pre-dosing at Day 1) in lesional Treatment Areas Assessment (TAA) at Days 8, 15 and 22, for area randomized to topically applied niclosamide 2%, as compared with vehicle.
Change from baseline in skin barrier and biomarker levels at Day 22, for area randomized to topically applied niclosamide 2%, as compared with vehicle.

### Inclusion Criteria

Patients were considered eligible for participation in the study if they met all the following inclusion criteria at the screening and baseline (pre-dosing at Day 1) visits, unless specified otherwise:
1. Man or woman 18 years of age or older at the time of consent.
2. Patient has clinically confirmed diagnosis of active atopic dermatitis, according to Hanifin and Rajka criteria (Hanifin et al. “Diagnostic feature of atopic dermatitis”, Acta. Derm. Ven. vol 92, (suppl): 44-47, 1980, see Table 7 below).
3. Patient has at least a 6-month history of atopic dermatitis and had no significant flares in atopic dermatitis for at least 4 weeks before screening (information obtained from medical chart or patient's physician, or directly from the patient).
4. Patient has moderate atopic dermatitis at baseline (pre-dosing at Day 1), as defined by an IGA of 3.
5. Patient has at least two areas of atopic dermatitis (excluding face, scalp, genitals, hands, and feet) of at least 3×3 cm; with a TSS of at least 5 at baseline (Day 1) for each treatment area. These areas should be at least 2 cm apart.
6. For patient (man and woman) involved in any sexual intercourse that could lead to pregnancy, patient agrees that an effective contraceptive method will be used, from at least 4 weeks before baseline (Day 1) until at least 4 weeks after the last study product administration. Effective contraceptive methods include hormonal contraceptives (combined oral contraceptive, patch, vaginal ring, injectable, or implant), intrauterine devices or intrauterine systems, vasectomy, tubal ligation, or a barrier method of contraception (male condom, female condom, cervical cap, diaphragm, contraceptive sponge) in conjunction with spermicide. Note: Hormonal contraceptives must have been on a stable dose for at least 4 weeks before baseline (Day 1).

Note: Woman of nonchildbearing potential is as follows:
Woman who has had surgical sterilization (hysterectomy, bilateral oophorectomy, or bilateral salpingectomy)
Woman ≥40 years of age who has had a cessation of menses for at least 12 months and a follicle-stimulating hormone (FSH) test confirming nonchildbearing potential (refer to laboratory reference ranges for confirmatory levels) or cessation of menses for at least 24 months without FSH levels confirmed
7. For woman of childbearing potential, has had a negative serum pregnancy test at screening and negative urine pregnancy test at baseline (Day 1).
8. Patient is willing to participate and is capable of giving informed consent. Note: Consent must be obtained prior to any study-related procedures.

### Exclusion Criteria

Patients were not considered to be eligible for participation in the study if they met any of the following criteria at the screening and baseline (Day 1) visits, unless specified otherwise:
1. Patient is a woman who is breastfeeding, pregnant, or who is planning to become pregnant during the study.
2. Patient has clinically infected atopic dermatitis.
3. Patient has a Fitzpatrick's Skin Phototype >5.
4. Presence of any tattoos, scratches, open sores, excessive hair, or skin damages in the target lesion areas that in the opinion of the investigator may interfere with study evaluations.
5. Patient is known to have immune deficiency or is immunocompromised.
6. Patient has a history of cancer or lymphoproliferative disease within 5 years prior to baseline (Day 1). Patients with successfully treated nonmetastatic cutaneous squamous cell or basal cell carcinoma and/or localized carcinoma in situ of the cervix are not to be excluded.
7. Patient had a major surgery within 8 weeks prior to baseline (Day 1) or has a major surgery planned during the study.
8. Patient has any clinically significant medical condition or physical/laboratory/vital signs abnormality that would, in the opinion of the investigator, put the patient at undue risk or interfere with interpretation of study results.
9. Patient has a known history of chronic infectious disease (e.g., hepatitis B, hepatitis C, or infection with human immunodeficiency virus).
10. Patient has used hydroxyzine or diphenhydramine within 1 week prior to Day 1.
11. Patient has used dupilumab within 12 weeks prior to Day 1.
12. Patient has received any nonbiological investigational product or device within 4 weeks prior to Day 1
13. Patient has used crisaborole and any other topical PDE-4 inhibitor within 4 weeks prior to Day 1.
14. Patient has used doxepin within 1 week prior to Day 1.
15. Patient has used topical products containing urea on target areas within 1 week prior to baseline (Day 1).
16. Patient used nonurea-containing emollient anywhere on the body from 1 day before Day 1.
17. Patient has used systemic antibiotics within 2 weeks or topical antibiotics on target areas within 1 week prior to baseline (Day 1).
18. Patient has used any topical medicated treatment for atopic dermatitis within 1 week prior to baseline (Day 1), including, but not limited to, topical corticosteroids, calcineurin inhibitors, tars, bleach, antimicrobials, medical devices, and bleach baths.
19. Patient has used systemic treatments (other than biologics) that could affect atopic dermatitis less than 4 weeks prior to baseline (Day 1) (e.g., retinoids, calcineurin inhibitors, methotrexate, cyclosporine, hydroxycarbamide [hydroxyurea], azathioprine, oral/injectable corticosteroids). Note: Intranasal corticosteroids and inhaled corticosteroids for stable medical conditions are allowed if patient has been on a stable dose for at least 4 weeks prior to baseline (Day 1) and will continue usage at the same dose for the duration of the study. Eye drops containing corticosteroids are allowed.
20. Patient has received any marketed or investigational biological agent within 12 weeks or 5 half-lives (whichever is longer) prior to baseline (Day 1).
21. Patient has excessive sun exposure, is planning a trip to a sunny climate, or has used tanning booths within 4 weeks prior to baseline (Day 1), or is not willing to minimize natural and artificial sunlight exposure during the study. Use of sunscreen products and protective apparel are recommended when exposure cannot be avoided.
22. Patient has a known or suspected allergy to niclosamide or any component of the formulation to be tested.
23. Patient has a known history of clinically significant drug or alcohol abuse in the last year prior to baseline (Day 1).
24. Patient has a history of an allergic reaction or significant sensitivity to lidocaine or other local anaesthetics.
25. Patient has a history of hypertrophic scarring or keloid formation in scars or suture sites.
26. Patient is taking anticoagulant medication, such as heparin, low molecular weight (LMW)-heparin, warfarin, antiplatelets (nonsteroidal anti-inflammatory drugs [NSAIDs] and low-dose aspirin ≤31 mg will not be considered antiplatelets), or has a contraindication to skin biopsies.

### Diagnosis of AD

Diagnosis of AD in a subject used the criteria according to Hanifin et al, ibid and set out in the Description of the present application. To be diagnosed with AD the subject should have at least three of the Major Criteria and at least three of the Minor Criteria

### Treatment

The study involved a comparison of the niclosamide topical composition with a matching vehicle, administered topically once daily for 3 weeks, without occlusion, at 5 mg/cm²/day (application areas 5×5 cm). The niclosamide formulation and placebo vehicle were applied on two separate target lesions of atopic dermatitis (lesions of at least 3×3 cm that are at least 2 cm apart, excluding the face, scalp, genitals, hands, and feet). As the chosen target lesion areas were expected to have a significant effect on outcomes, it was considered important to make a considerable effort to ensure selection of treatment areas with similar severity to reduce bias. Subjects came to the study site for all study product (active or vehicle) applications.

### Efficacy Assessments

Clinical evaluations of atopic dermatitis were performed by an experienced and qualified dermatologist (board certified or equivalent) or other suitably qualified and experienced designee. To assure consistency and reduce variability, the same assessor performed all assessments on a given subject whenever possible.

### Eczema Area and Severity Index

The Eczema Area and Severity Index (EASI) were assessed pre-dosing (Day 1). It quantifies the severity of the atopic dermatitis based on both lesion severity and the percentage of body surface area (BSA) affected. The EASI is a composite score ranging from 0 to 72 that takes into account the degree of erythema, induration/infiltration (papules), excoriation, and lichenification (each scored from 0 to 3 separately) for each of four body regions, with adjustment for the percentage of BSA involved for each body region and for the proportion of the body region to the whole body. The EASI score calculation is set out in the description.

### Body Surface Area

The overall BSA affected by atopic dermatitis was evaluated (from 0% to 100%) pre-dosing (Day 1). For example, one subject's palm represents 1% of total BSA.

### Total Sign Score (TSS)

The lesional TSS on each of the two treatment areas was assessed pre-dosing (Day 1). It quantifies the severity of a subject's atopic dermatitis based on severity of erythema, edema/papulation, oozing/crusting, excoriation, lichenification, and dryness (each scored from 0 to 3, separately). The lesional TSS is a composite score ranging from 0 to 18. A detailed procedure of lesional TSS score calculation is set out in the description. To be eligible for this study, subjects had a TSS score of pre-dosing (Day 1) for each treatment area.

### Treatment Areas Assessment (TAA)

The lesional TAA on each of the two treatment areas was assessed at the visits specified in Table 3. The lesional TAA grades the severity of disease (each area scored from 0 to 5, separately). More details on the lesional TAA score assessment is provided in the description.

### Skin Biopsies

Skin barrier and inflammation biomarker levels were determined from lesional skin biopsies from application areas. All subjects had a total of three skin biopsies: one biopsy at Day 1 and 2 biopsies at Day 22 (one where niclosamide was applied and one where the vehicle was applied).

Subjects who discontinued from the study but had completed at least the Day 15 visit, received treatment applications on Days 13 and 14, and received at least 12 applications up to Day 14, inclusively, had a biopsy taken as was planned for Day 22.

The skin biopsy samples were analysed by immunohistochemistry (IHC), and by gene expression studies by RT-PCR using TaqMan Low Density Array (TLDA), and by microarray using Affymetrix U133A Plus 2. The immunohistochemistry (IHC) was used to analyse cell biomarkers. The methodologies as disclosed by Guttman-Yassky et al, “Major differences in inflammatory dendritic cells and their products distinguish atopic dermatitis from psoriasis”, The Journal of Allergy and Clinical Immunology, vol. 119, issue 5, pages 1210-1217, 2007, were followed except for that U133A Plus 2-set Gene Chip probe arrays was used instead of U95A-set Gene Chip probe arrays.

### TLDA Data Analysis

Expression values (threshold cycle [Ct]) were normalized to Rplp0 by negatively transforming the Ct values to −dCt (IL17A was normalized to hARP, as analysed by qPCR). The undetected −dCt values were estimated for each gene as the 20% of the minimum across all samples. qRT-PCR expression data were modelled using a mixed effect model with Visit and Treatment Area as a fixed effect and a random intercept for each patient. This formulation intrinsically models the within patient correlation structure as in the case of a paired t-test. This approach introduces less bias than restricting the analysis for those patients who completed the study. Contrasts were used to estimate the fold changes with treatment within each treatment group and conduct hypothesis testing.

### Microarray Data Analysis

Experimental design: The hybridization strategy was in concordance with experimental design principles, by for example keeping all samples from the same patient in the same date, and always include samples from every treatment arm/group.

Quality Control and Pre-processing: Quality control of microarray chips were carried out using standard QC metrics and R package microarray Quality Control. Expression measures were obtained using GCRMA algorithm (Wu & Irizarry, 2004). Several visual and modelling techniques were used to elucidate if batch effect existed. Principal Component analysis plots were used to detect if any evident batch effect existed. If such batch effects were found, they were adjusted using Combat, an empirical Bayes method for adjusting data for batch effects that is robust to outliers in small sample sizes (Johnson, Li, & Rabinovic, 2007). The implementation of Combat by package sva was used.

Probe-sets with at least 5% samples with expression larger than 3 (in log 2-scale) were kept for further analysis. Expression values were modelled using mixed-effect models with fixed factors Visit and Treatment Area and a random effect for each patient. Fold changes for the comparisons of interest were estimated and hypothesis testing was conducted on such comparisons using contrasts under the general framework for linear models in limma package. The inter-replicate correlation was computed by Duplicate Correlation function and the linear model was estimated by ImFit. P-values from the moderated (paired) t-test were adjusted for multiple hypotheses using the Benjamini-Hochberg procedure, which controls for FDR.

### Statistical Analysis—TSS and TAA

Continuous variables were summarized in tables and included the number of patients, mean, standard deviation, median, minimum, and maximum. Categorical variables were presented in tables as frequencies and percentages.

The comparison between the treatment groups for change from baseline in TSS at Day 22, was done using a paired Student t-test. The difference between treatments was estimated and presented along with a 95% confidence interval.

The other endpoints involving change from baseline were analysed using the same approach as described above.

### Analysis Sets

Data from subjects who are randomized were included in the Intent to Treat (ITT) analysis set. Data from subjects who received at least one administration of study treatment on each lesion were included in the modified ITT (MITT) analysis set. Data were analyzed according to the treatment group to which the subject was randomized.

The Per Protocol (PP) analysis set I included data from subjects who were randomized, had no significant protocol deviations effecting the efficacy assessment, and had evaluable data for the primary endpoint.

The Safety analysis set (SAF) was defined as data from subjects who received at least one administration of the study product. Analysis was performed according to the actual treatment subjects received.

### Efficacy Analysis—Lesional TSS at Day 22

The comparison between the treatment groups for change from baseline in lesional TSS at Day 22, was done using a paired Student t-test. The difference between treatments was estimated and presented along with a 95% confidence interval. Descriptive statistics for the baseline, Day 22 and change from baseline to Day 22 lesional TSS were presented for lesions treated with niclosamide and vehicle in the MITT population. Ninety-five percent confidence intervals (CIs) using a t-distribution were determined for the point estimates for change from baseline in each treatment group. Descriptive statistics and a 95% CI using a t-distribution were also provided for the difference between the change from baseline in lesional TSS for the niclosamide and placebo lesions. Subjects with missing TSS at Day 22 were included in the analysis using a last observation carried forward imputation for the missing data. Analyses of the primary efficacy endpoint were repeated in the PP population.

Efficacy endpoints include TSS at Day 1 (pre-dosing), 815 and 22, and TAA at Days 1 (pre-dosing), 8, 15 and 22. Analyses of endpoints were conducted in the same manner as described above for the other efficacy endpoint.

### Efficacy Analysis—Biomarker/Clinical Score Correlation Analysis

The variables that were used for the correlation analysis were the clinical score (Total Sign Score (TSS) and Target Area Assessment (TAA)) of Day 22 and Baseline (Day 1) and the normalized biomarker expression values that were analysed with qRT-PCR (TLDA) and for the same days. The absolute change with treatment at Day 22 were calculated for each patient and each treatment. For the assessment of pairwise correlation the Spearman correlation coefficient was used. It is a non-parametric measure of rank correlation. The significant correlations were plotted with the respective linear regression line, a confidential interval of 95% and its respective rho (spearman coefficient, R) and p value. For this correlation analysis biomarkers were selected that showed significant changes in qRT-PCR and/or microarray. The correlation analysis was made on qRT-PCR data only except for the immune cells were IHC data was taken.

The same procedure was applied when analysing the correlation of individual scores and biomarker expression values. For this analysis biomarkers were taken that showed significant correlation to TSS or/and TAA. The correlation analysis was made on qRT-PCR data only.

### Biomarkers

Immune effectors (herein also referred to as biomarkers) included in the immunohistochemistry (IHC) and in the gene expression analysis using qRT-PCR were grouped as shown in Table 7:

TABLE 7

| Group | Biomarkers |
|---|---|
| General inflammation | MMP12 |
| Proliferation | KRT16 |
| Th-1 related | CXCL10, IFNg, IL12A, |
| | CXCL9 |
| Th2-related | IL13, IL10, IL33, TSLPR, |
| | IL31, IL5, IL9 |
| | CCL17, CCL22, CCL18, |
| | CCL26 |
| Innate immunity | IL6, IL8, IL17C, IL1B |
| Skin barrier/Terminal | LOR, FLG, PPL |
| differentiation | |
| Th17/Th22-related | S100A8, S100A12, S100A7, |
| | S100A9, IL22 |
| Th17-related | IL17A, IL17F, IL23A, CAMP, |
| | IL19, IL12B |
| | DEFB4A/DEFB4B, CXCL1, |
| | CXCL2, CCL20, PI3 |
| T-Cell/NK cell activation | IL15, IL15RA, IL2, CCL5 |
| T regulatory cell | FOXP3 |
| Dendritic cells | CD11c, FceR1 |
| Langerhans Cells | Langerin |
| T cells | CD3 |

Thymic stromal lymphopoietin protein receptor (TSLP-R) is the receptor for the proinflammatory cytokine thymic stromal lymphopoietin (TSLP).

CD3 (cluster of differentiation 3) is a biomarker for T cells.

FOXP3 (also known as scurfin) is a biomarker for a subpopulation of T cells called regulatory T cells (also known as suppressor T cells).

As mentioned above, the biomarkers that showed significant changes in qRT-PCR (TLDA) expression analysis were selected for correlation analysis with TSS and TAA.

Further biomarkers were included in the microarray analysis, see Tables 14-17.

### Results

### Skin Thickness at Day 22

No differences in skin thickness were found following treatment with with 2% niclosamide compared to baseline and compared to vehicle.

### Expression Levels of Biomarkers at Day 22 and Correlation Versus Total Severity Score (TSS) and Target Area Assessment (TAA)

Biomarkers were analysed by qRT-PCR or microarray in the skin biopsies taken at Day 1 and at Day 22 as described hereinbefore.

The results for all biomarkers analysed by qRT-PCR are presented in Tables 8-13.

The results for all biomarkers analysed by microarray are presented in Tables 14-16.

TABLE 8
qRT-PCR - all Biomarkers results

| | | Vehicle | | Niclosamide | | Niclosamide | |
|---|---|---|---|---|---|---|---|
| | Biomarker | vs. Predose | | vs. Predose | | vs. Vehicle | |
| General Inflammation | MMP12 | −1.72 | + | −3.27 | *** | −1.90 | + |
| Innate Immunity Markers | IL1B | −1.63 | * | −1.64 | * | −1.01 | |
| Innate Immunity Markers | IL8 | −2.59 | * | −3.78 | ** | −1.46 | |
| Innate Immunity Markers | IL6 | −1.89 | * | −2.34 | ** | −1.24 | |
| Innate Immunity Markers | IL17C | −1.38 | | −3.24 | *** | −2.35 | ** |
| Skin barrier/Proliferation | KRT16 | −2.43 | ** | −3.88 | *** | −1.60 | |
| T cell Activation Marker | IL2 | 1.13 | | −1.20 | | −1.36 | |
| T cell Activation Marker | CCL5 | −1.19 | | −1.22 | | −1.03 | |
| T cell Activation Marker | IL15 | −1.29 | * | −1.47 | *** | −1.14 | |
| T cell Activation Marker | IL15RA | −1.31 | ** | −1.46 | *** | −1.12 | |
| T regulatory cell Marker | FOXP3 | −1.22 | | −1.39 | * | −1.14 | |
| Skin barrier/Terminal | PPL | −1.11 | | −1.07 | | 1.04 | |
| Differentiation Marker | | | | | | | |
| Skin barrier/Terminal | FLG | 1.16 | | 1.50 | * | 1.30 | |
| Differentiation Marker | | | | | | | |
| Skin barrier/Terminal | LOR | 1.32 | | 1.67 | * | 1.27 | |
| Differentiation Marker | | | | | | | |
| TH1 related genes | IFNg | −1.32 | | −1.11 | | 1.19 | |
| TH1 related genes | IL12A | −1.09 | | −1.01 | | 1.09 | |
| TH1 related genes | CXCL9 | −1.75 | * | −1.51 | + | 1.16 | |
| TH1 related genes | CXCL10 | −1.49 | | −1.64 | * | −1.10 | |
| Th17 chemokine related genes | CCL20 | 1.15 | | −1.96 | ** | −2.25 | *** |
| Th17 chemokine related genes | CXCL2 | −1.56 | * | −1.89 | ** | −1.21 | |
| Th17 chemokine related genes | DEFB4A/ | −1.98 | + | −4.09 | *** | −2.06 | + |
| | DEFB4B | | | | | | |
| Th17 chemokine related genes | CXCL1 | −1.72 | + | −2.80 | *** | −1.63 | + |
| Th17 chemokine related genes | PI3 | −2.34 | * | −4.52 | *** | −1.93 | + |
| Th17 cytokine related genes | IL17F | −1.34 | | 1.02 | | 1.37 | |
| Th17 cytokine related genes | IL19 | −3.77 | * | −3.26 | * | 1.16 | |
| Th17 cytokine related genes | CAMP | −2.27 | * | −3.02 | *** | −1.33 | |
| Th17 cytokine related genes | IL12B | −1.42 | | −2.01 | * | −1.42 | |
| Th17 cytokine related genes | IL23A | −1.56 | * | −2.13 | *** | −1.37 | |
| Th17 cytokine related genes | IL17A | −1.60 | + | −2.23 | ** | −1.40 | |
| Th17/TH22 related genes | S100A7 | −1.38 | | −3.04 | *** | −2.20 | ** |
| Th17/TH22 related genes | IL22 | −1.45 | | −2.86 | ** | −1.97 | + |
| Th17/TH22 related genes | S100A9 | −1.76 | + | −3.47 | *** | −1.97 | * |
| Th17/TH22 related genes | S100A8 | −1.91 | + | −3.85 | *** | −2.02 | + |
| Th17/TH22 related genes | S100A12 | −2.03 | + | −4.29 | *** | −2.11 | + |
| Th2 related chemokines | CCL26 | 1.44 | * | −1.03 | | −1.49 | * |
| Th2 related chemokines | CCL18 | −1.21 | | −2.45 | *** | −2.02 | ** |
| Th2 related chemokines | CCL22 | −1.32 | | −1.95 | ** | −1.49 | + |
| Th2 related chemokines | CCL17 | −1.48 | | −2.02 | * | −1.37 | |
| Th2 related genes | IL33 | −1.21 | + | −1.08 | | 1.11 | |
| Th2 related genes | IL31 | −1.27 | | −1.85 | | −1.46 | |
| Th2 related genes | IL10 | −1.23 | | −1.85 | *** | −1.51 | ** |
| Th2 related genes | IL13 | −1.29 | | −2.06 | * | −1.59 | |
| Th2 related genes | IL5 | −1.62 | | −2.38 | * | −1.47 | |
| Th9 | IL9 | −1.62 | | −1.91 | | −1.17 | |
| Th2 related genes | TSLPR | −1.33 | | −1.82 | ** | −1.36 | |

*** (p < 0.001)
** (p < 0.01)
* (p < 0.05)
+ (p < 0.1)

TABLE 9
Biomarkers that changed significant with treatment
at Day 22 compared to Baseline (qRT-PCR)

| | | Niclosamide | |
|---|---|---|---|
| | Biomarker | vs Predose | |
| General Inflammation | Matrix Metallopeptidase 12 (MMP12) | −3.27 | *** |
| Innate Immunity Markers | Interleukin 1B (IL1B) | −1.64 | * |
| Innate Immunity Markers | Interleukin 8 (IL8) | −3.78 | ** |
| Innate Immunity Markers | Interleukin 6 (IL6) | −2.34 | ** |
| Innate Immunity Markers | Interleukin 17C (IL17C) | −3.24 | *** |
| Skin barrier/Proliferation | Keratin 16 (KRT16) | −3.88 | *** |
| T cell Activation Marker | Interleukin 15 (IL15) | −1.47 | *** |
| T cell Activation Marker | Interleukin 15RA (IL15RA) | −1.46 | *** |
| T regulatory Marker | Forkhead Box P3 (FOXP3) | −1.39 | * |
| Skin barrier/Terminal | Filaggrin (FLG) | 1.50 | * |
| Differentiation Marker | | | |
| Skin barrier/Terminal | Loricrin (LOR) | 1.67 | * |
| Differentiation Marker | | | |
| TH1 related genes | Chemokine (C-X-C Motif) Ligand 10 | −1.64 | * |
| | (CXCL10) | | |
| Th17 chemokine related genes | Chemokine (C-C Motif) Ligand 20 | −1.96 | ** |
| | (CCL20) | | |
| Th17 chemokine related genes | Chemokine (C-X-C Motif) Ligand 2 | −1.89 | ** |
| | (CXCL2) | | |
| Th17 chemokine related genes | Defensin Beta 4A/B (DEFB4A/DEFB4B) | −4.09 | *** |
| Th17 chemokine related genes | Chemokine (C-X-C Motif) Ligand 1 | −2.80 | *** |
| | (CXCL1) | | |
| Th17 chemokine related genes | Peptidase Inhibitor 3 (PI3) | −4.52 | *** |
| Th17 cytokine related genes | Interleukin 19 (IL19) | −3.26 | * |
| Th17 cytokine related genes | Cathelicidin Antimicrobial Peptide | −3.02 | *** |
| | (CAMP) | | |
| Th17 cytokine related genes | Interleukin 12B (IL12B) | −2.01 | * |
| Th17 cytokine related genes | Interleukin 23A (IL23A) | −2.13 | *** |
| Th17 cytokine related genes | Interleukin 17A (IL17A) | −2.23 | ** |
| Th17/TH22 related genes | S100 Calcium Binding Protein 7 | −3.04 | *** |
| | (S100A7) | | |
| Th17/TH22 related genes | Interleukin 22 (IL22) | −2.86 | ** |
| Th17/TH22 related genes | S100 Calcium Binding Protein 9 | −3.47 | *** |
| | (S100A9) | | |
| Th17/TH22 related genes | S100 Calcium Binding Protein 8 | −3.85 | *** |
| | (S100A8) | | |
| Th17/TH22 related genes | S100 Calcium Binding Protein 12 | −4.29 | *** |
| | (S100A12) | | |
| Th2 related chemokines | Chemokine (C-C Motif) Ligand 18 | −2.45 | *** |
| | (CCL18) | | |
| Th2 related chemokines | Chemokine (C-C Motif) Ligand 22 | −1.95 | ** |
| | (CCL22) | | |
| Th2 related chemokines | Chemokine (C-C Motif) Ligand 17 | −2.02 | * |
| | (CCL17) | | |
| Th2 related genes | Interleukin 10 (IL10) | −1.85 | *** |
| Th2 related genes | Interleukin 13 (IL13) | −2.06 | * |
| Th2 related genes | Interleukin 5 (IL5) | −2.38 | * |
| Th2 related genes | TSLPR | −1.82 | ** |

***(p < 0.001)
**(p < 0.01)
*(p < 0.05)
+(p < 0.1)

TABLE 10
Biomarkers that are significant changed with
treatment compared to vehicle (qRT-PCR)

| | | Niclosamide | |
|---|---|---|---|
| | Biomarker | vs vehicle | |
| Innate Immunity Markers | Interleukin 17C (IL17C) | −2.35 | ** |
| Th17 chemokine related genes | Chemokine (C-C Motif) Ligand 20 | −2.25 | *** |
| | (CCL20) | | |
| Th17/TH22 related genes | S100 Calcium Binding Protein 7 | −2.20 | ** |
| | (S100A7) | | |
| Th17/TH22 related genes | S100 Calcium Binding Protein 9 | −1.97 | * |
| | (S100A9) | | |
| Th2 related chemokines | Chemokine (C-C Motif) Ligand 26 | −1.49 | * |
| | (CCL26) | | |
| Th2 related chemokines | Chemokine (C-C Motif) Ligand 18 | −2.02 | ** |
| | (CCL18) | | |
| Th2 related genes | Interleukin 10 (IL10) | −1.51 | ** |

***(p < 0.001)
**(p < 0.01)
*(p < 0.05)
+(p < 0.1)

TABLE 11
Biomarkers that are significant changed to Baseline
and vehicle with Niclosamide (qRT-PCR)

| | | Niclosamide | | Niclosamide | |
|---|---|---|---|---|---|
| | | vs. Predose | | vs. vehice | |
| Innate Immunity Markers | Interleukin 17C (IL17C) | −3.24 | *** | −2.35 | ** |
| Th17 chemokine related genes | Chemokine (C-C Motif) Ligand 20 | −1.96 | ** | −2.25 | *** |
| | (CCL20) | | | | |
| Th17/TH22 related genes | S100 Calcium Binding Protein 7 | −3.04 | *** | −2.20 | ** |
| | (S100A7) | | | | |
| Th17/TH22 related genes | S100 Calcium Binding Protein 9 | −3.47 | *** | −1.97 | * |
| | (S100A9) | | | | |
| Th2 related chemokines | Chemokine (C-C Motif) Ligand 18 | −2.45 | *** | −2.02 | ** |
| | (CCL18) | | | | |
| Th2 related genes | Interleukin 10 (IL10) | −1.85 | *** | −1.51 | ** |

***(p < 0.001)
**(p < 0.01)
*(p < 0.05)
+(p < 0.1)

TABLE 12
Significant correlations of biomarker expression
(based on qRT-PCR/IHC data) to TSS at Day 22

| | | TSS_p |
|---|---|---|
| | TSS | value |
| S100A8 | 0.829 | 0.000 |
| S100A7 | 0.794 | 0.000 |
| KRT16 | 0.769 | 0.000 |
| S100A9 | 0.761 | 0.000 |
| S100A12 | 0.694 | 0.000 |
| PI3 | 0.689 | 0.000 |
| IL13 | 0.681 | 0.000 |
| IL22 | 0.670 | 0.000 |
| DEFB4A/DEFB4B | 0.601 | 0.001 |
| CCL17 | 0.585 | 0.001 |
| MMP12 | 0.545 | 0.002 |
| LOR | −0.516 | 0.004 |
| CCL22 | 0.502 | 0.006 |
| IL17A | 0.479 | 0.009 |
| IL19 | 0.472 | 0.010 |
| CD11c_Dermis | 0.460 | 0.012 |
| IL8 | 0.420 | 0.023 |
| FLG | −0.406 | 0.029 |
| CXCL1 | 0.377 | 0.044 |
| CAMP | 0.374 | 0.046 |

TABLE 13
Significant correlation of biomarker expression
(based on qRT-PCR data) to TAA at Day 22

| | | TAA_p |
|---|---|---|
| | TAA | value |
| KRT16 | 0.694 | 0.000 |
| S100A7 | 0.667 | 0.000 |
| S100A8 | 0.658 | 0.000 |
| S100A9 | 0.643 | 0.000 |
| IL13 | 0.641 | 0.000 |
| IL22 | 0.632 | 0.000 |
| CCL17 | 0.599 | 0.001 |
| MMP12 | 0.590 | 0.001 |
| S100A12 | 0.553 | 0.002 |
| PI3 | 0.526 | 0.003 |
| DEFB4A/DEFB4B | 0.518 | 0.004 |
| IL19 | 0.456 | 0.013 |
| IL8 | 0.500 | 0.006 |
| CCL22 | 0.440 | 0.017 |
| LOR | −0.432 | 0.019 |
| FLG | −0.408 | 0.028 |

TABLE 14
Biomarker expression levels that changed significant
with treatment compared to Baseline (Microarray)

| | |
|---|---|
| | Niclosamide |
| Biomarker | vs Predose |

| | | | |
|---|---|---|---|
| Dendritic cells | CD86 | −1.60 | *** |
| General Inflammation | CCL19 | −1.56 | ** |
| General Inflammation | IL24 | −2.19 | ** |
| General Inflammation | MMP12 | −2.81 | ** |
| skin barrier lipids | ACOX2 | 1.40 | * |
| skin barrier lipids | ACSL1 | 1.21 | *** |
| unsorted | ANXA6 | −1.24 | * |
| skin barrier lipids | CDSN | 1.14 | ** |
| skin barrier lipids | CERS3 | 1.13 | * |
| skin barrier lipids | CLN8 | 1.15 | * |
| skin barrier lipids | ELOVL3 | 1.81 | * |
| skin barrier lipids | EREG | 1.19 | * |
| skin barrier lipids | FA2H | 1.50 | * |
| skin barrier lipids | FAR2 | 1.48 | * |
| skin barrier lipids | KRT79 | 1.91 | * |
| skin barrier lipids | PNPLA3 | 1.91 | ** |
| skin barrier lipids | PPL | 1.06 | * |
| skin barrier, epidermal | ACER1 | 1.94 | *** |
| differentiation | | | |
| skin barrier, epidermal | ANXA9 | 1.56 | *** |
| differentiation | | | |
| skin barrier, epidermal | CLDN1 | 1.17 | * |
| differentiation | | | |
| skin barrier, epidermal | CLDN23 | 1.42 | ** |
| differentiation | | | |
| skin barrier, epidermal | DGAT2 | 1.33 | *** |
| differentiation | | | |
| skin barrier, epidermal | DHCR7 | 1.12 | * |
| differentiation | | | |
| skin barrier, epidermal | FAXDC2 | 1.46 | ** |
| differentiation | | | |
| skin barrier, epidermal | FLG | 1.17 | * |
| differentiation | | | |
| skin barrier, epidermal | KRT23 | 1.26 | *** |
| differentiation | | | |
| skin barrier, epidermal | KRT77 | 1.43 | ** |
| differentiation | | | |
| skin barrier, epidermal | SCEL | 1.43 | *** |
| differentiation | | | |
| unsorted | SPTLC3 | 1.44 | *** |
| skin barrier, epidermal | TJP3 | 1.51 | ** |
| differentiation | | | |
| T cell activation | CCR7 | −1.51 | ** |
| T cell activation | CD2 | −1.56 | * |
| T cell activation | CD28 | −1.72 | * |
| T cell activation | CD3D | −1.63 | ** |
| T cell activation | CD3G | −1.62 | * |
| Th1 | CCL2 | −1.77 | ** |
| Th1 | CCR1 | −1.90 | *** |
| Th1 | CCR2 | −1.37 | * |
| Th1 | IFNGR2 | −1.13 | ** |
| Th1 | IL12RB2 | −1.21 | * |
| Th1 | IL2RA | −1.27 | * |
| Th1 | IRF1 | −1.37 | * |
| Th17 | CCR6 | −1.33 | * |
| Th17 | CXCL1 | −2.83 | ** |
| Th17 | CXCL2 | −1.81 | * |
| Th17 | IL6R | −1.25 | *** |
| Th17 | LCN2 | −1.62 | * |
| Th17 | PI 3 | −3.13 | *** |
| Th17 | STAT3 | −1.13 | * |
| Th17 | IL37 | 1.69 | * |
| TH17 | TNFSF4 | −1.64 | *** |
| Th17/Th22 | S100A12 | −3.62 | *** |
| Th17/Th22 | S100A7 | −1.17 | * |
| Th17/Th22 | S100A8 | −1.45 | ** |
| Th17/Th22 | S100A9 | −2.81 | *** |
| Th17/Th22 | S100P | −1.35 | * |
| Th17/Th22 | SERPINB1 | −1.29 | *** |
| Th17/Th22 | SERPINB4 | −1.54 | * |
| Th2 | CCL13 | −1.40 | ** |
| Th2 | CCL18 | −1.70 | ** |
| Th2 | CCL22 | −1.83 | ** |
| Th2 | CCR5 | −1.40 | ** |
| Th2 | IL4R | −1.80 | *** |
| Th2 | IL7R | −1.58 | * |
| unsorted | IL1F10 | 1.98 | *** |

TABLE 15
Biomarker expression (microarray) that changed
significant with treatment compared to vehicle

| | |
|---|---|
| | Vehicle vs. |
| Biomarker | Niclosamide |

| | | | |
|---|---|---|---|
| unsorted | CCL23 | 1.63 | * |
| unsorted | IL26 | 1.78 | * |
| unsorted | ACOT2 | −1.35 | * |
| skin barrier lipids | ACOX2 | −1.40 | * |
| skin barrier lipids | ELOVL3 | −1.66 | ** |
| skin barrier lipids | FA2H | −1.57 | * |
| skin barrier lipids | FAR2 | −1.47 | * |
| skin barrier lipids | KRT79 | −2.00 | * |
| skin barrier lipids | PNPLA3 | −1.79 | ** |
| unsorted | PPARG | −1.55 | * |
| skin barrier, epidermal | DGAT2 | −1.19 | * |
| differentiation | | | |
| skin barrier, epidermal | FAXDC2 | −1.29 | * |
| differentiation | | | |
| unsorted | SPTLC3 | −1.30 | ** |
| Th1 | CCL2 | 1.56 | * |
| Th1 | CCR1 | 1.55 | ** |
| Th1 | IFNGR2 | 1.10 | * |
| Th1 | STAT1 | 1.13 | * |
| Th17 | CCL20 | 2.00 | * |
| Th17 | CCR6 | 1.31 | * |
| Th17 | CXCL1 | 2.10 | * |
| Th17 | LCN2 | 1.69 | * |
| Th17 | PI 3 | 1.87 | * |
| Th17 | STAT3 | 1.16 | ** |
| TH17 | TNFSF4 | 1.35 | * |
| Th17/Th22 | S100A12 | 2.30 | * |
| Th17/Th22 | S100A9 | 1.88 | * |
| Th2 | CCL13 | 1.32 | * |
| Th2 | CCL18 | 1.66 | * |
| Th2 | CCL26 | 1.69 | * |
| Th2 | IL4R | 1.29 | * |
| unsorted | IL1F10 | −1.79 | *** |

TABLE 16
Biomarkers that are significant changed compared to
Baseline and vehicle with treatment (Microarray)

| | | Niclosamide | | Vehicle vs. | |
|---|---|---|---|---|---|
| | Biomarker | vs Predose | | Niclosamide | |
| skin barrier lipids | ACOX2 | 1.40 | * | −1.40 | * |
| skin barrier lipids | ELOVL3 | 1.81 | * | −1.66 | * |
| skin barrier lipids | FA2H | 1.50 | * | −1.57 | * |
| skin barrier lipids | FAR2 | 1.48 | * | −1.47 | * |
| skin barrier lipids | KRT79 | 1.91 | * | −2.00 | * |
| skin barrier lipids | PNPLA3 | 1.91 | ** | −1.79 | ** |
| skin barrier, epidermal | DGAT2 | 1.33 | *** | −1.19 | * |
| differentiation | | | | | |
| skin barrier, epidermal | FAXDC2 | 1.46 | ** | −1.29 | * |
| differentiation | | | | | |
| unsorted | SPTLC3 | 1.44 | *** | −1.30 | ** |
| Th1 | CCL2 | −1.77 | ** | 1.56 | * |
| Th1 | CCR1 | −1.90 | *** | 1.55 | ** |
| Th1 | IFNGR2 | −1.13 | ** | 1.10 | * |
| Th17 | CCR6 | −1.33 | * | 1.31 | * |
| Th17 | CXCL1 | −2.83 | ** | 2.10 | * |
| Th17 | LCN2 | −1.62 | * | 1.69 | * |
| Th17 | PI 3 | −3.13 | *** | 1.87 | * |
| Th17 | STAT3 | −1.13 | * | 1.16 | ** |
| TH17 | TNFSF4 | −1.64 | *** | 1.35 | * |
| Th17/Th22 | S100A12 | −3.62 | *** | 2.30 | * |
| Th17/Th22 | S100A9 | −2.81 | *** | 1.88 | * |
| Th2 | CCL13 | −1.40 | ** | 1.32 | * |
| Th2 | CCL18 | −1.70 | ** | 1.66 | * |
| Th2 | IL4R | −1.80 | *** | 1.29 | * |
| unsorted | IL1F10 | 1.98 | *** | −1.79 | *** |

### CONCLUSIONS

As evident from the above presented results, significant changes from baseline (pre-dosing at Day 1) were found for certain immune effectors in the biopsies taken at Day 22.

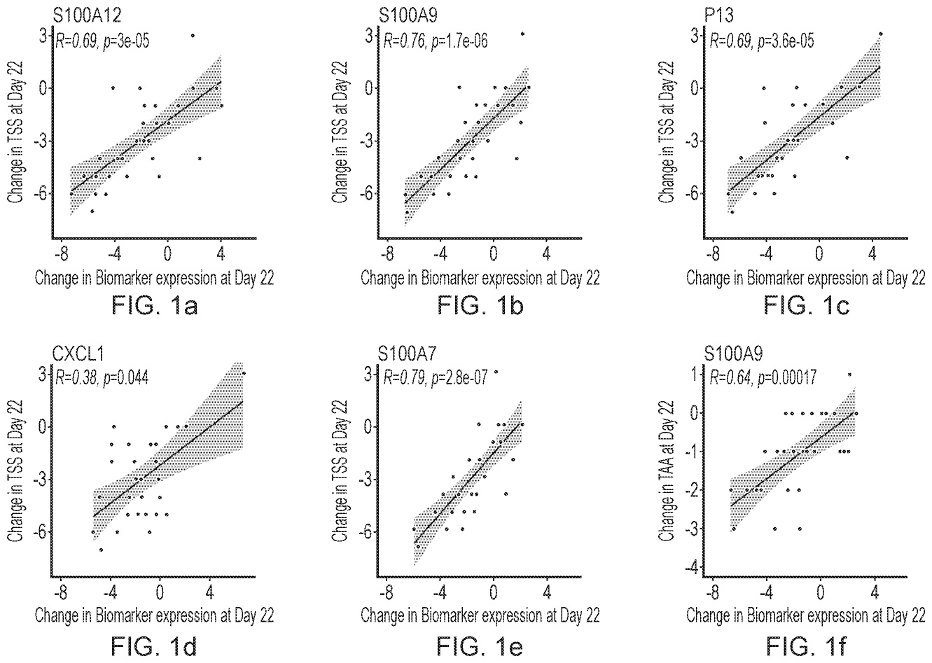

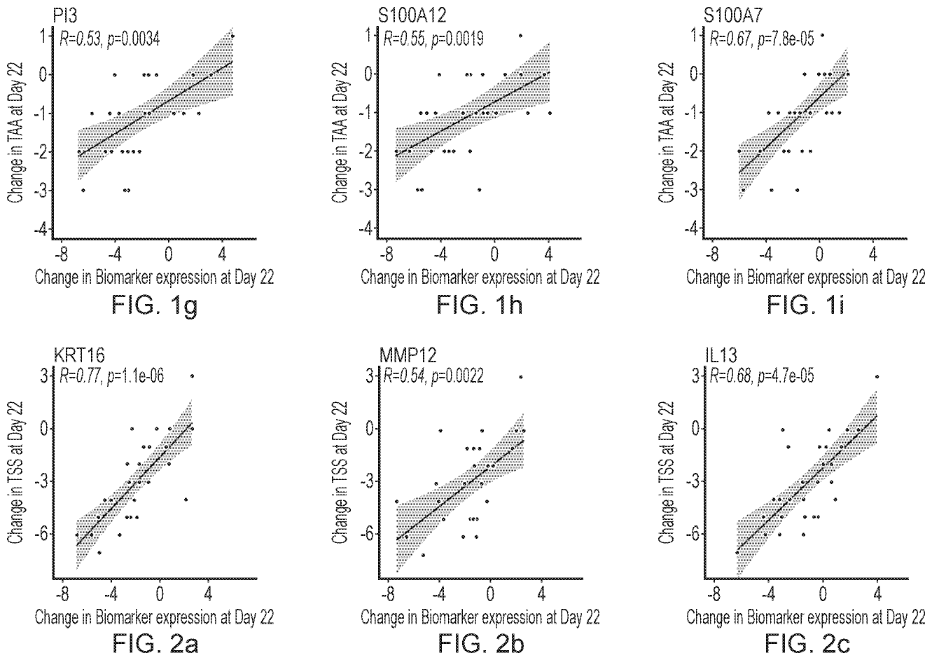

S100A12 was found to be significantly downregulated at Day 22 following topical administration of 2% niclosamide compared to baseline (−3.62) and compared to vehicle (−2.30), p<0.05). S100A12 was found to be significantly correlated with TSS and TAA. Results are shown in FIGS. 1a and 1 h, respectively. The graphs show the correlation of change in biomarker expression at Day 22 compared to baseline to change in TSS at Day 22.

S100A9 was found to be significantly downregulated at Day 22 following topical administration of 2% niclosamide compared to baseline (−2.81) and compared to vehicle (−1.88) (p<0.05). S100A9 was found to be significantly correlated with TSS and TAA.

Results are shown in FIGS. 1b and 1f respectively. The graphs show the correlation change in biomarker expression at Day 22 compared to baseline to change in TSS at Day 22.

PI3 was found to be significantly downregulated at Day 22 following topical administration of 2% niclosamide compared to baseline (−3.13) and compared to vehicle (−1.87) (p<0.05). PI3 was found to be significantly correlated to TSS and TAA. Results are shown in FIGS. 1c and 1g respectively. The graphs show the correlation of change in biomarker expression at Day 22 compared to baseline to change in TSS at Day 22.

CXCL1 was found to be significantly downregulated at Day 22 following topical administration of 2% niclosamide compared to baseline (−2.83) and compared to vehicle (−2.10) (p<0.05). CXCL1 was found to be significantly correlated to TSS. Results are shown in FIG. 1d. The graphs show the correlation of change in biomarker expression at Day 22 compared to baseline to change in TSS at Day 22.

S100A7 was found to be significantly downregulated at Day 22 following topical administration of 2% niclosamide compared to baseline (−3.04) and compared to vehicle (−2.20) (p<0.05). S100A7 was found to be significantly correlated to TSS and TAA. Results are shown in FIGS. 1e and 1i, respectively. The graphs show the correlation of change in biomarker expression at Day 22 compared to baseline to change in TSS at Day 22.

Thus, S100A12, S100A9, PI3, S100A7 and CXCL1 were all shown to be significantly downregulated in expression compared to baseline as well as vehicle and were all found to be clinically correlated to TSS.

Among these biomarkers that showed significant change compared to vehicle and baseline, S100A7 and S100A9 were found to have the highest correlations to TSS and S100A7 and S100A9 to were found to have the highest correlations to TAA.

The levels of the biomarkers listed in Table 9 above and analysed by qRT-PCR were also found to have changed significantly at Day 22 compared to baseline following topical administration of 2% niclosamide.

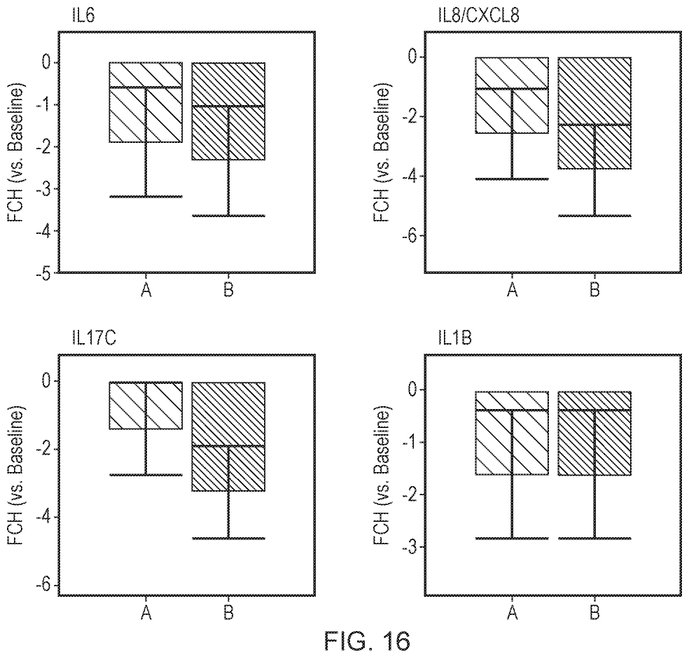

Results are shown in FIGS. 16-25, where A denotes vehicle and B denotes niclosamide at Day 22 compared to baseline.

FIG. 16 shows changes in biomarkers (IL6, IL8, IL17C, IL1B) associated with innate immunity.

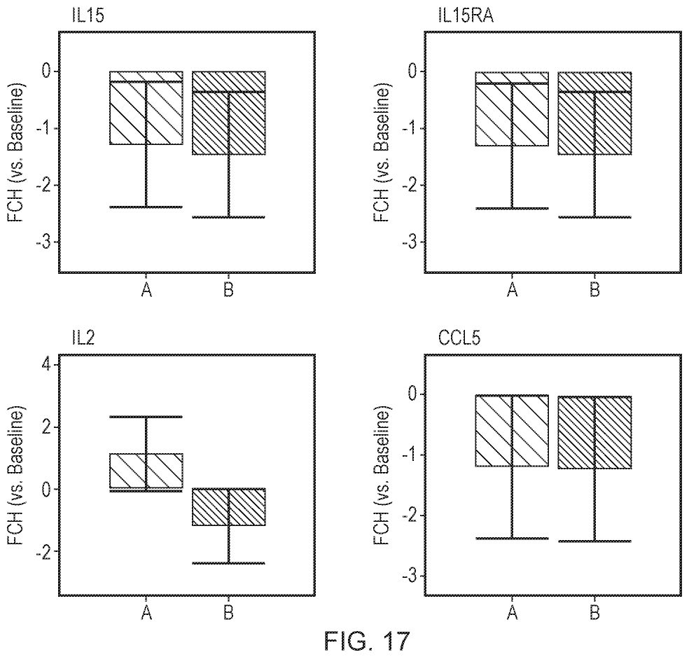

FIG. 17 shows changes in biomarkers (IL15, IL15RA, IL2, CCL5) associated with T cell activation.

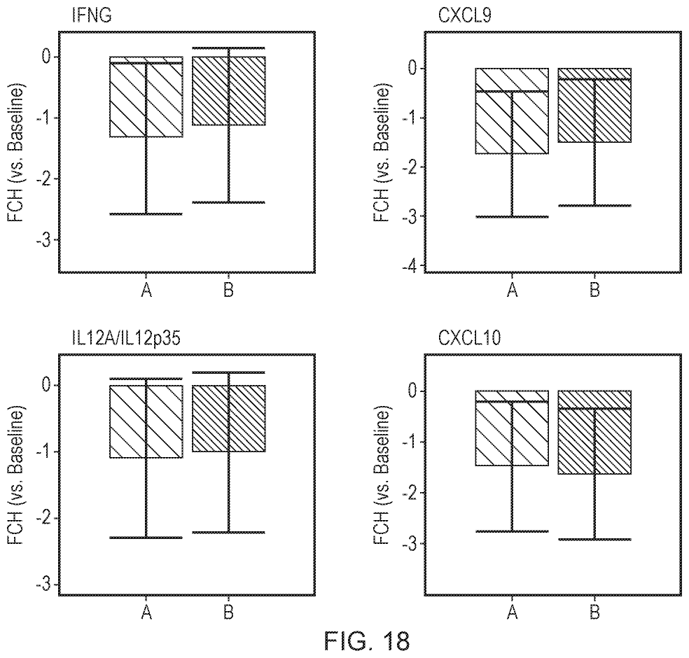

FIG. 18 shows changes in biomarkers (IFNG, CXCL9, IL12A/IL12p35, CXCL10) associated with Th1 related genes.

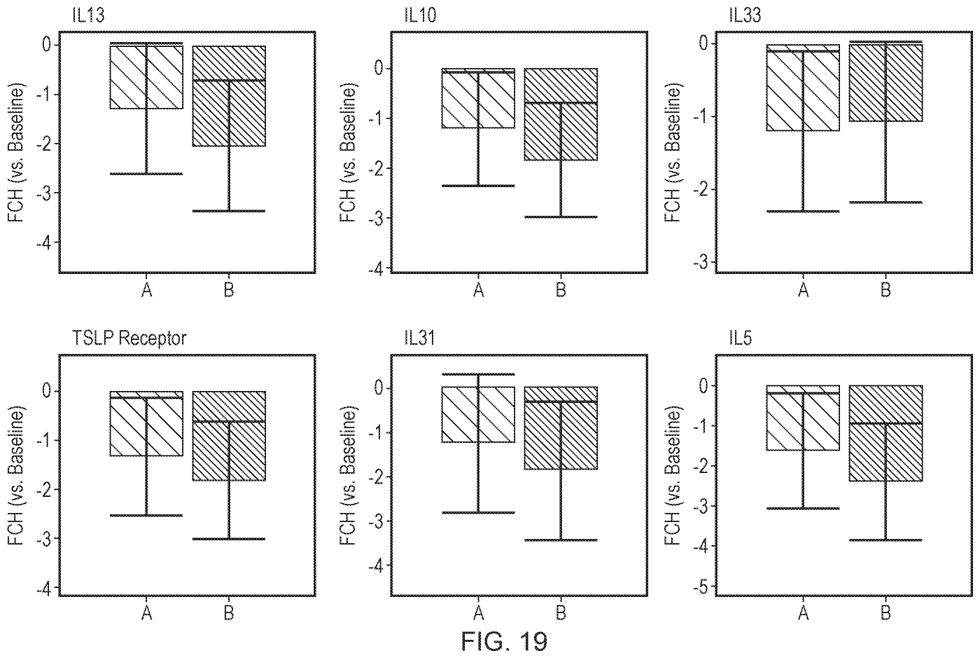

FIG. 19 shows changes in biomarkers (IL13, IL10, IL33, TSLP-R, IL31, IL5) associated with Th2 related genes.

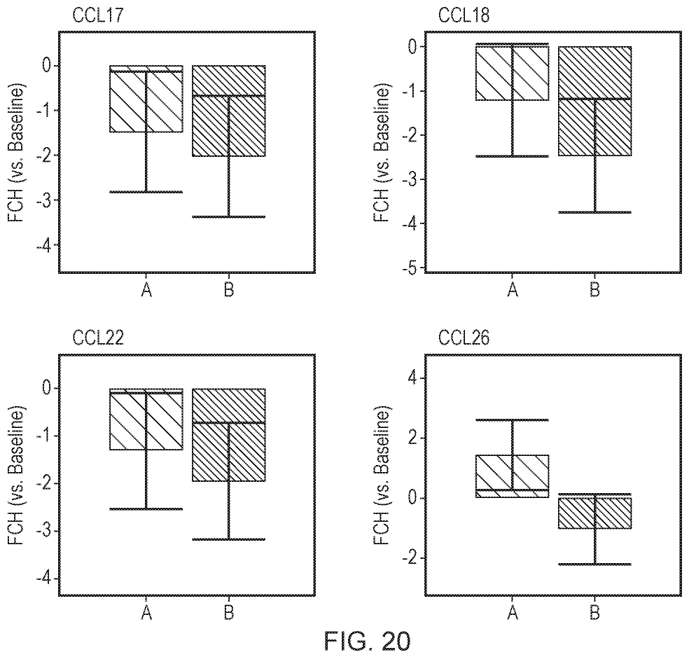

FIG. 20 shows changes in biomarkers (CCL17, CCL18, CCL22, CCL26) associated with Th2 related chemokines.

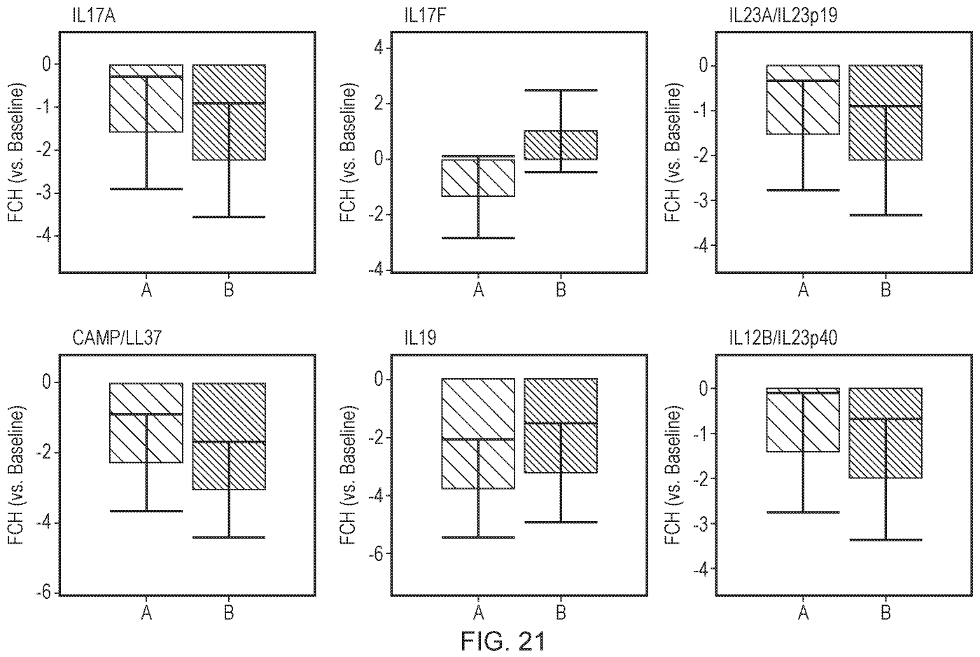

FIG. 21 shows changes in biomarkers (IL17A, IL17F, IL23A/IL23p19, CAMP/LL37, IL19, IL12B/IL23p40) associated with Th17 cytokine related genes.

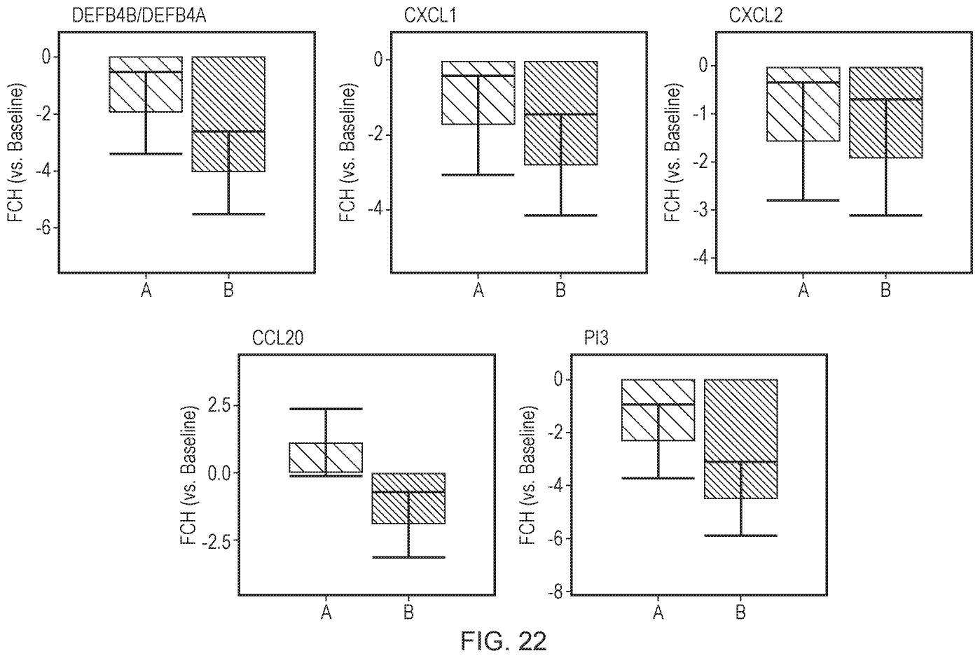

FIG. 22 shows changes in biomarkers (DEFB4A/DEFB4B, CXCL1, CXCL2, CCL20, PI3) associated with Th17 chemokine related genes.

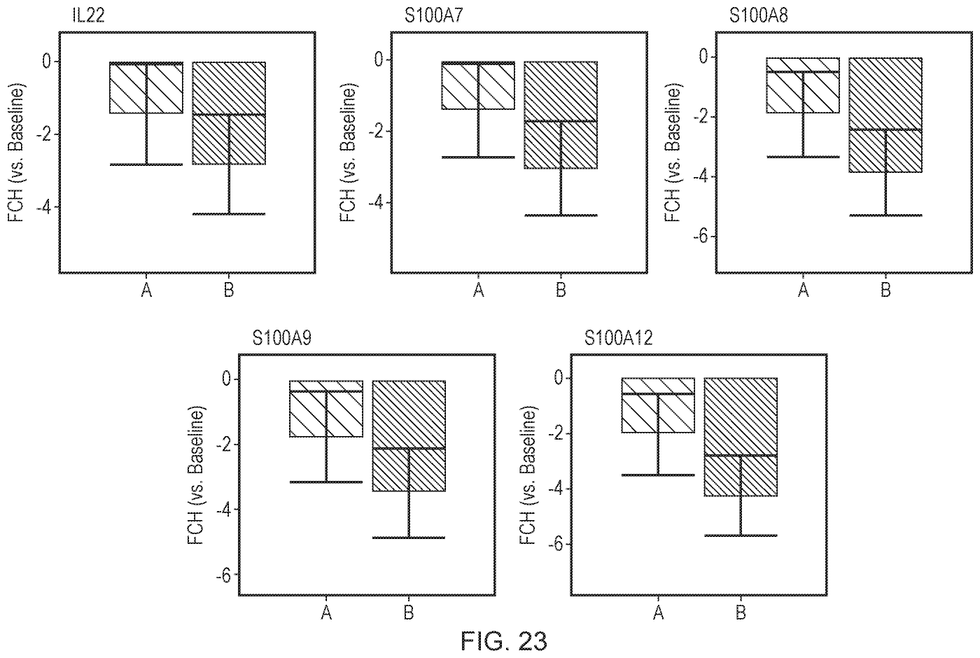

FIG. 23 shows changes in biomarkers (IL22, S100A7, S100A8, S100A9, S100A12) associated with Th17/Th22 related genes.

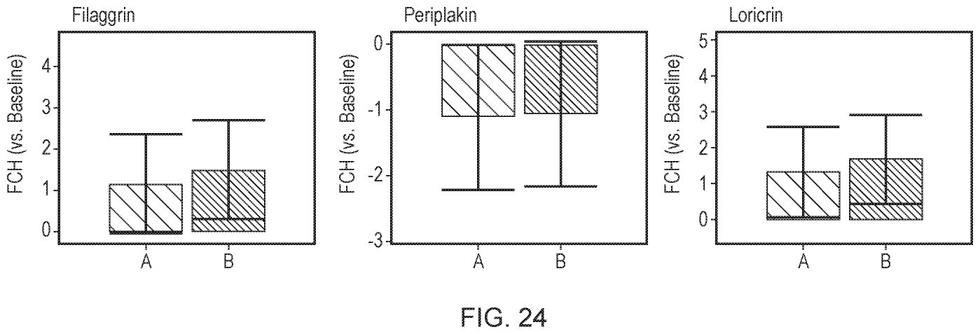

FIG. 24 shows changes in biomarkers (FLG, PPL, LOR) associated with terminal differentiation.

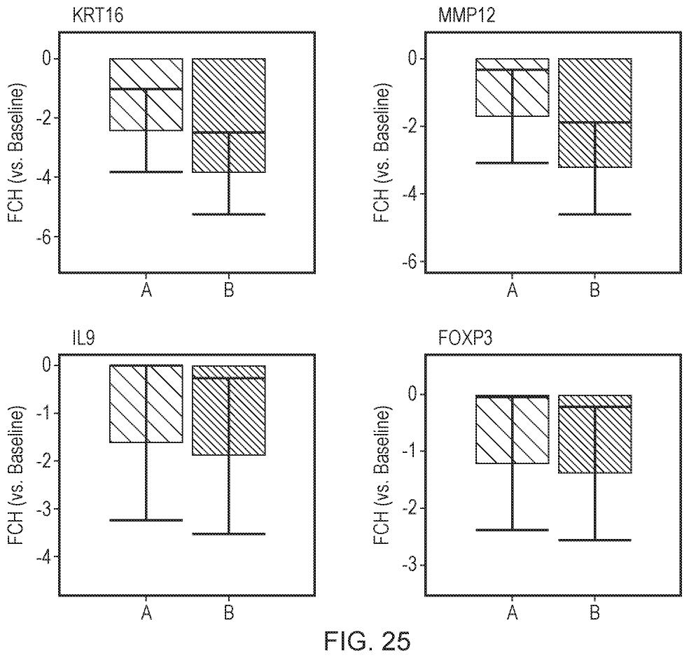

FIG. 25 shows changes in biomarkers (KRT16) associated with proliferation, general inflammation (MMP12), Th9 (IL9) and T regulatory cells (FOXP3).

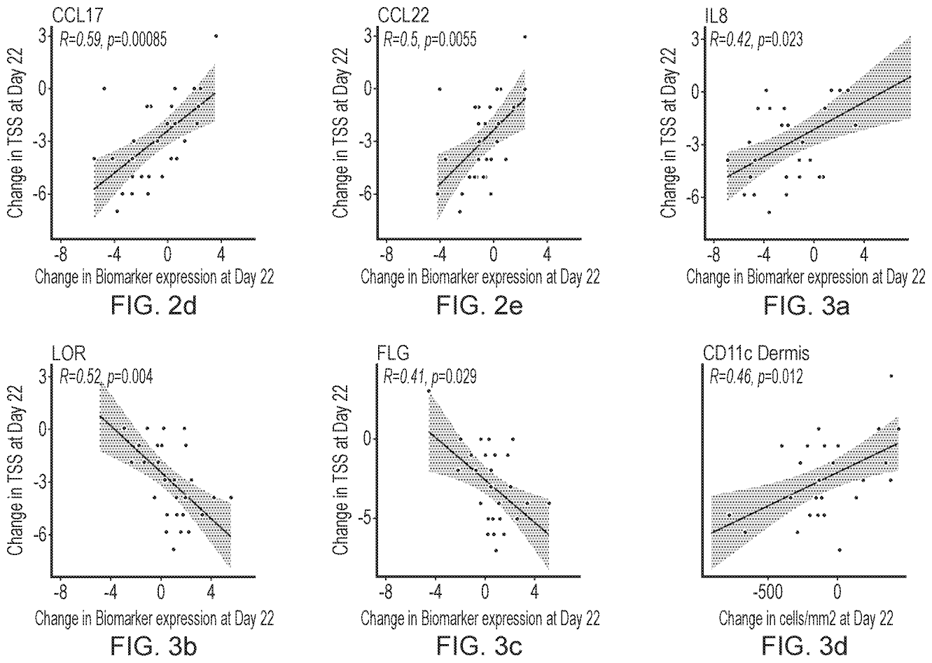

Correlations between change in biomarker expression versus TSS are shown in FIGS. 2-5. The graphs show the correlation of biomarker change at Day 22 compared to baseline to change in TSS at Day 22.

FIGS. 2a and 2b show biomarkers (KRT16, MMP12) associated with proliferation/general inflammation.

FIGS. 2c, 2d and 2e show biomarkers (IL13, CCL17, CCL22) associated with Th2 related chemokines and cytokines.

FIG. 3a show biomarkers (IL8) associated with innate immunity.

FIGS. 3b and 3c show biomarkers (LOR, FLG) associated with skin barrier/terminal differentiation.

FIG. 3d show biomarkers (CD11c Dermis) associated with dendritic cells.

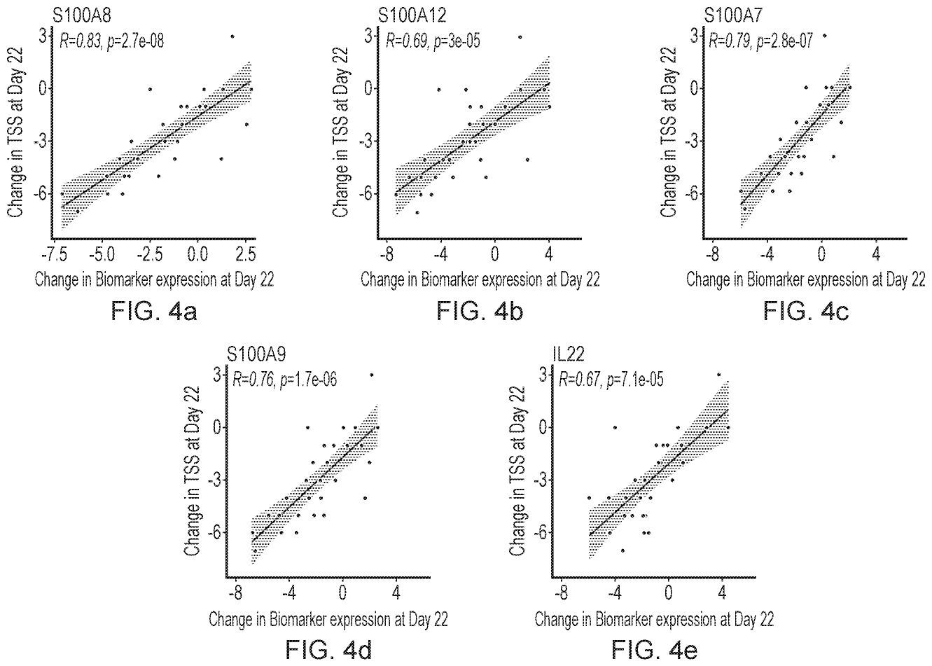

FIGS. 4a-4e show biomarkers (S100A8, S100A12, S100A7, S100A9, IL22) associated with Th17/Th22 related chemokines and cytokines.

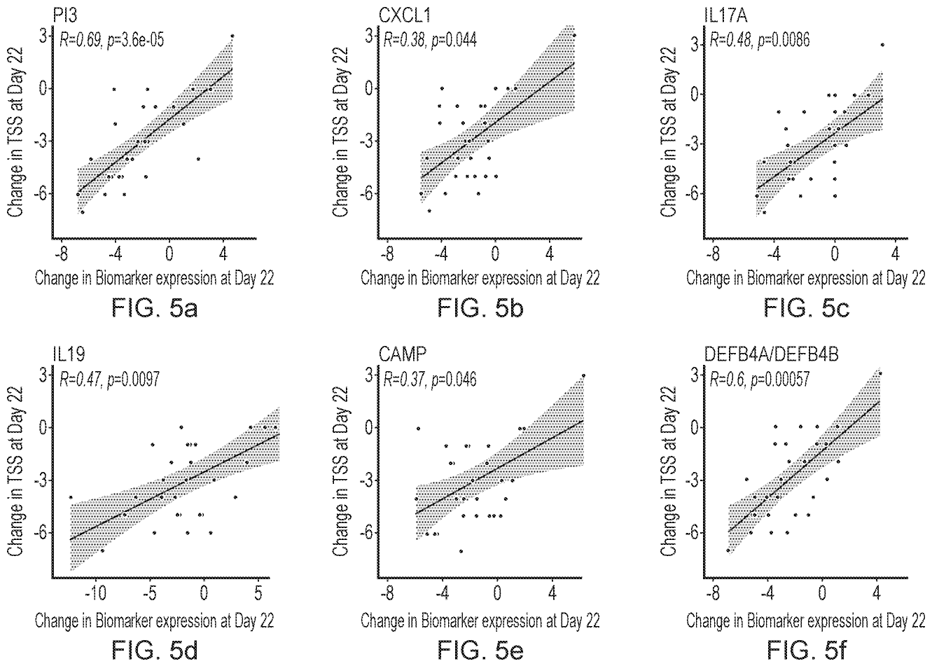

FIGS. 5a-5f show biomarkers (PI3, CXCL1, IL17A, IL19, CAMP, DEFB4A/DEFB4B) associated with Th17 related chemokines and cytokines.

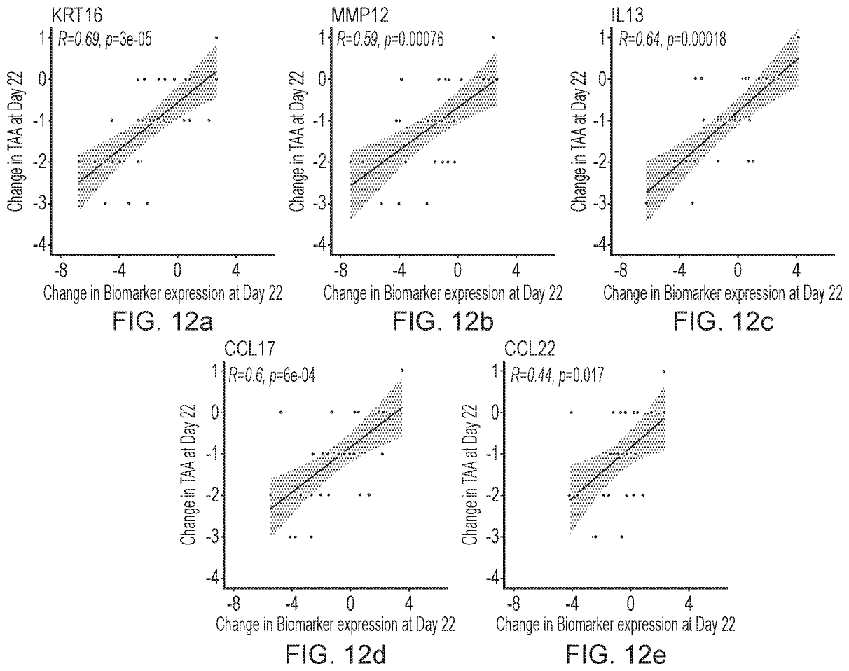

Correlations between change in biomarker expression versus TAA are shown in FIGS. 12-15. The graphs show the biomarker change at Day 22 compared to baseline.

FIGS. 12a and 12b show biomarkers (KRT16, MMP12) associated with proliferation/general inflammation.

FIGS. 12c, 12d and 12e show biomarkers (IL13, CCL17, CCL22) associated with Th2 related chemokines and cytokines.

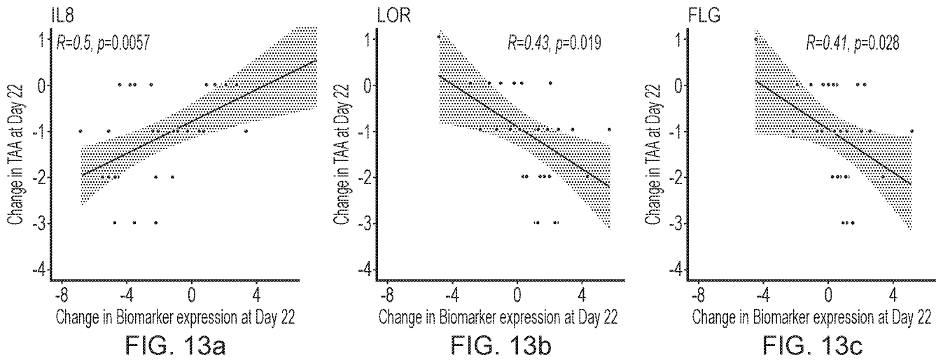

FIG. 13a show biomarkers (IL8) associated with innate immunity.

FIGS. 13b and 13c show biomarkers (LOR, FLG) associated with skin barrier/terminal differentiation.

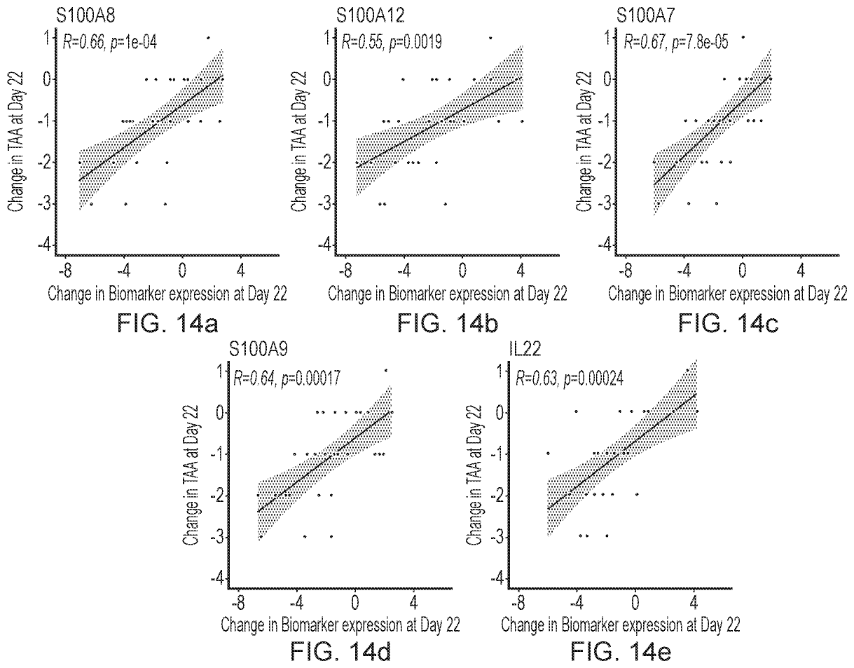

FIGS. 14a-14e show biomarkers (S100A8, S100A12, S100A7, S100A9, IL22) associated with Th17/Th22 related chemokines and cytokines.

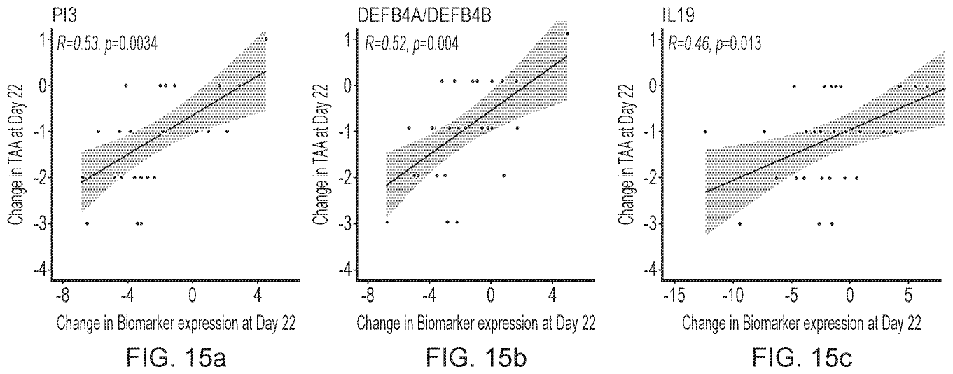

FIGS. 15a-15c show biomarkers (PI3, DEFB4A/DEFB4B, IL19) associated with Th17 related chemokines and cytokines.

All these biomarkers analyzed with qRT-PCR except for LOR and FLG were found to have decreased significantly at Day 22 following topical administration of 2% niclosamide compared to baseline (see Tables 8-13).

LOR and FLG were found to have increased significantly at Day 22 following topical administration of 2% niclosamide compared to baseline, see FIGS. 13b and 13c. LOR and FLG are involved in terminal differentiation of epidermal cells and an increased expression of any one of these proteins is associated with a better skin barrier. Increased expression of LOR induced by topical niclosamide was shown to be associated with an improvement of signs and symptoms of AD.

Also, some skin barrier proteins and lipids analyzed with microarray (see Tables 14-16) were found to have increased significantly at Day 22 following topical administration of 2% niclosamide compared to baseline and vehicle. Skin barrier lipids that were found to have increased compared to baseline and vehicle, by using the microarray analysis, were ACOX2, EVOLV3, FA2H, FAR2, KRT79, PNPLA3. Skin barrier proteins that were found to have increased compared to baseline and vehicle, by using the microarray analysis, were DGAT2 and FAXDC2.

The increased expression of structural skin barrier proteins and lipids indicate that niclosamide are useful for treatment of an inflammatory skin condition associated with skin barrier dysfunction, e.g. an inflammatory skin condition associated with skin barrier deficiency in one or more skin barrier molecules, such as AD, by improving the skin barrier function.

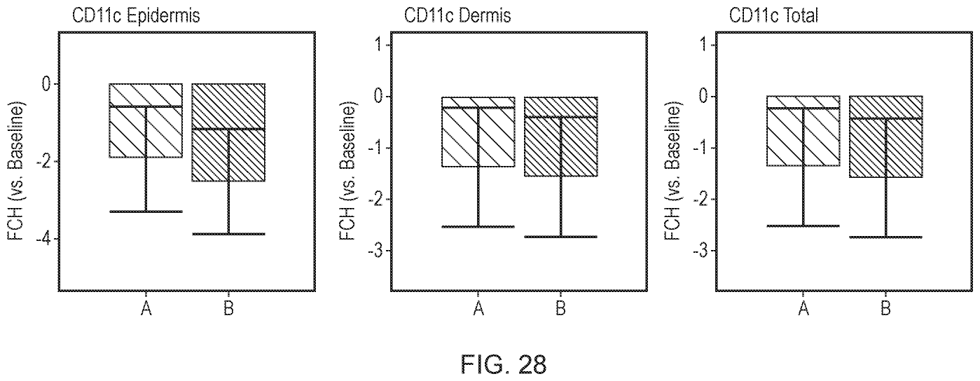

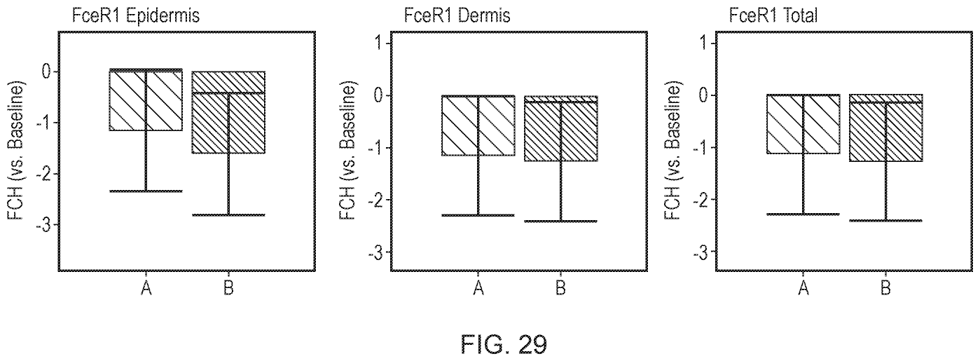

Treatment with 2% niclosamide was shown to decrease inflammation and immune cell infiltrates compared to baseline (pre-dosing at Day 1). Significant reductions in inflammatory cells (dendritic cells: CD11c, FceR1 in epidermis, and Langerhans cells: langerin/CD207) compared to baseline (pre-dosing at Day 1) in patients topically treated with 2% niclosamide were found (FIGS. 27-29). CD11c Dermis was significantly changed in expression level compared to baseline and clinically correlated to TSS (see FIG. 28).

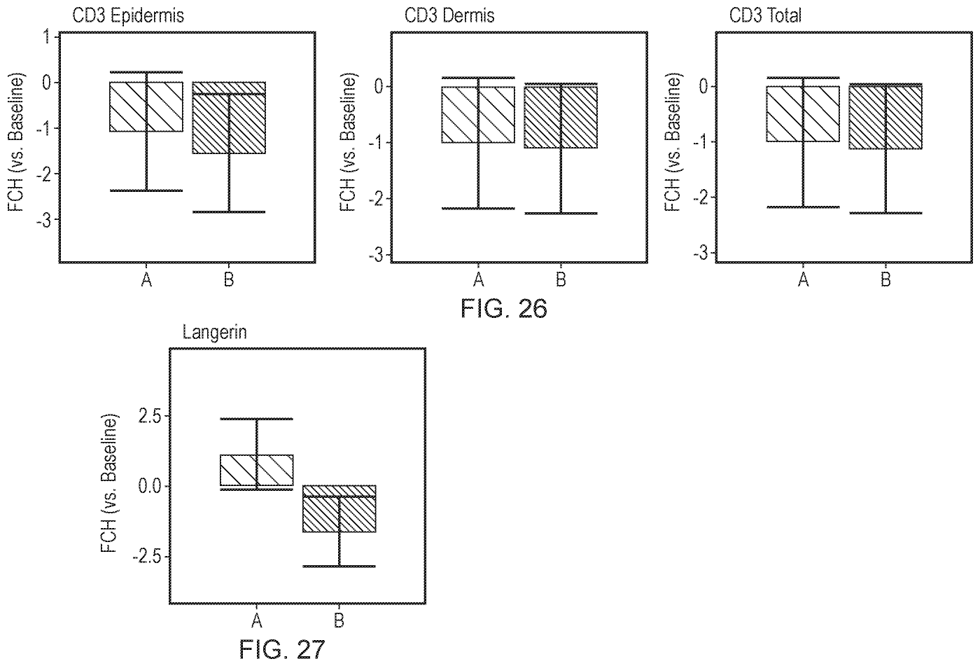

No significant change of the total amount of T cells (i.e. T cells expressing CD3D and CD3G) was found (in dermis and epidermis) compared to baseline (pre-dosing at Day 1) in patients topically treated with 2% niclosamide, see FIG. 26.

In patients treated with 2% niclosamide, there were significant changes from baseline in certain inflammatory markers including those of general inflammation (MMP12), proliferation (KRT16), innate immunity (IL6, IL17C, IL8, IL1B), terminal differentiation (FLG, LOR), T-Cell/NK cell activation (IL15, IL15RA), Th1 pathway (CXCL10), Th2 pathway (CCL17, CCL18, CCL22, IL10, IL13, IL5, TSLPR), Th17 pathway (IL17A, IL23p19, IL23A, CCL20, CXCL1, CXCL2, PI3, DEFB4A/DEFB4B, PI3, IL12B), general inflammation (MMP12), T regulatory cells (FOXP3), Th17/TH22 pathway (S100A7, IL22, S100A8, S100A9, S100A12).

The results show that topical administration of niclosamide significantly downregulates expression of immune effectors associated with the Th1, Th2, Th17 and Th22-type immune responses, including innate immune effectors.

Th2, Th17, Th22 responses are crucial in the inflammatory loop of AD. The reduced expression of these key biomarkers and the direct correlation of these biomarkers to clinical signs and symptoms strongly support use of niclosamide for treatment of AD.

Th1 and Th17 responses are key drivers in psoriasis. The strong reduction of key markers of Th1 and Th17 driven inflammation highlight the utility of niclosamide for treatment of psoriasis.

Inflammatory mediators involved with T-cell activation, Th1, Th2 and Th17 are overexpressed in rosacea patients. These inflammatory mediators are down regulated by niclosamide supporting the use of niclosamide to treat rosacea.

Brunner et al (The Journal of Allergy and Clinical Immunology, Volume 139, Issue 4, Supplement, Pages S65-S76, 2017) discloses the effects of dupilumab on lesional AD skin, such as reduction in expression of Th2-associated molecules, such as CCL17, CCL18, and CCL26, and decrease in mediators associated with T_{H}17 and T_{H}22 responses. The biomarker profile in lesional AD skin treated with dupilumab is shown in Figure 4 of the Brunner reference.

Reference is here also made to Hamilton, Jennifer D., et al. “Dupilumab improves the molecular signature in skin of patients with moderate-to-severe atopic dermatitis.” Journal of Allergy and Clinical Immunology 134.6 (2014): 1293-1300; and Brunner, Patrick M., et al. “A mild topical steroid leads to progressive anti-inflammatory effects in the skin of patients with moderate-to-severe atopic dermatitis.” Journal of Allergy and Clinical Immunology 138.1 (2016): 169-178.

The similarities in biomarker profiles between niclosamide and dupilumab, triamcinolone acetonide and cyclosporine indicate that niclosamide are useful as an anti-inflammatory treatment of inflammatory skin conditions, such as AD (see Table 17).

The similarities in biomarker profiles between niclosamide and cyclosporine also indicate that niclosamide would be useful as an anti-inflammatory treatment of inflammatory ocular conditions, such as DED (see Table 17).

Moreover, a significant correlation was found between clinical score and some of the inflammation markers as further discussed hereinafter.

### Correlation of Individual Scores Versus TSS

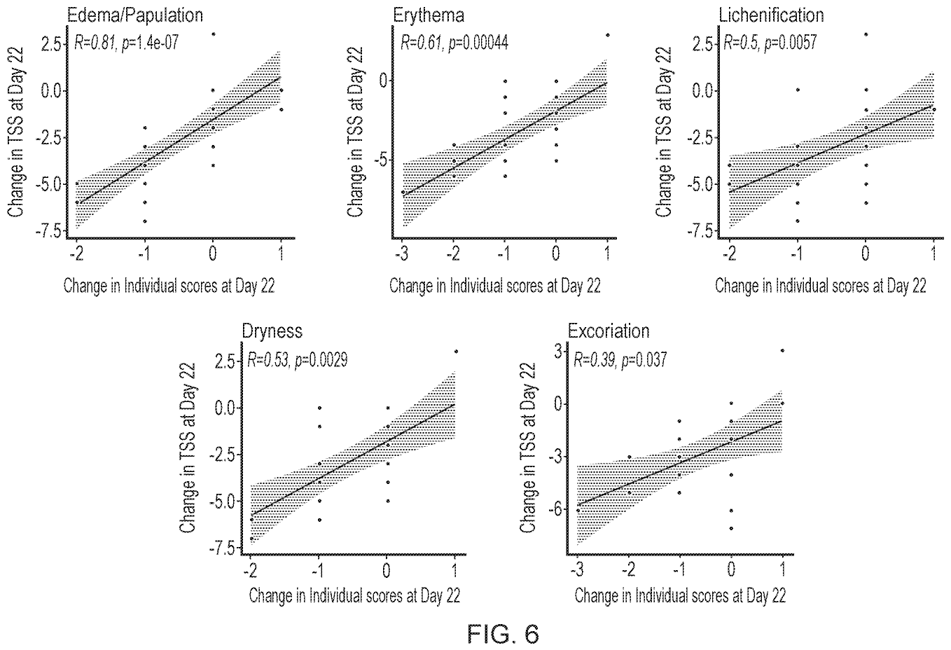

FIG. 6 shows the correlation between individual scores (erythema, edema/papulation, oozing/crusting, excoriation, lichenification and dryness) and TSS.

Oozing/Crusting had mostly a TSS of 0 at baseline.

It was found that edema/papulation, erythema, lichenification and dryness were drivers of the descending change in the TSS score.

Correlation of Biomarker Expression Versus Individual Scores

### Table 18a-e: Significant Correlation of Biomarker Expression (qRT-PCR) to Individual Scores at Day 22

TABLE 18a

| | Dryness | Dryness_p value |
|---|---|---|
| IL13 | 0.413 | 0.026 |

TABLE 18b

| | Erythema | Erythema_p value |
|---|---|---|
| S100A7 | 0.528 | 0.003 |
| S100A9 | 0.521 | 0.004 |
| KRT16 | 0.519 | 0.004 |
| IL13 | 0.508 | 0.005 |
| S100A8 | 0.504 | 0.005 |
| DEFB4A/DEFB4B | 0.489 | 0.007 |
| PI3 | 0.457 | 0.013 |
| CCL17 | 0.432 | 0.019 |
| S100A12 | 0.412 | 0.026 |
| IL22 | 0.411 | 0.027 |
| MMP12 | 0.382 | 0.041 |

TABLE 18c

| | Excoriation | Excoriation_p value |
|---|---|---|
| IL8 | 0.458 | 0.012 |

TABLE 18d

| | Edema/ | Edema/ |
|---|---|---|
| | Papulation | Papulation_p value |
| IL13 | 0.661 | 0.000 |
| S100A7 | 0.656 | 0.000 |
| S100A8 | 0.621 | 0.000 |
| KRT16 | 0.603 | 0.001 |
| IL22 | 0.576 | 0.001 |
| S100A9 | 0.568 | 0.001 |
| S100A12 | 0.542 | 0.002 |
| CCL17 | 0.531 | 0.003 |
| PI3 | 0.480 | 0.008 |
| MMP12 | 0.479 | 0.009 |
| CCL22 | 0.472 | 0.010 |
| DEFB4A/DEFB4B | 0.393 | 0.035 |
| IL19 | 0.389 | 0.039 |
| LOR | −0.375 | 0.045 |

TABLE 18e

| | Lichenification | Lichenification_p value |
|---|---|---|
| IL22 | 0.551 | 0.002 |
| LOR | −0.481 | 0.008 |
| S100A7 | 0.443 | 0.016 |
| S100A8 | 0.419 | 0.024 |
| S100A12 | 0.393 | 0.035 |
| S100A9 | 0.378 | 0.043 |
| DEFB4A/DEFB4B | 0.377 | 0.044 |

### Table 19: Significant Correlations of Change in Biomarker Expression to Change in Clinical Symptoms of the Biomarkers that Showed Significant Change in Expression Compared to Baseline and Vehicle (in qRT-PCR and Microarray)

TABLE 19

| | S100A7 | S100A9 | S100A8 | PI3 | CXCL1 |
|---|---|---|---|---|---|
| TSS | 0.794 | 0.761 | 0.829 | 0.689 | 0.377 |
| TSS_p.value | 0.000 | 0.000 | 0.000 | 0.000 | 0.044 |
| TAA | 0.667 | 0.643 | 0.658 | 0.526 | |
| TAA_p.value | 0.000 | 0.000 | 0.000 | 0.003 | |
| Erythema | 0.528 | 0.521 | 0.504 | 0.457 | |
| Erythema_p.value | 0.003 | 0.004 | 0.005 | 0.013 | |
| Edema/Papulation | 0.656 | 0.568 | 0.621 | 0.480 | |
| Edema/Papulation_p.value | 0.000 | 0.001 | 0.000 | 0.008 | |
| Lichenification | 0.443 | 0.378 | 0.419 | | |
| Lichenification_p.value | 0.016 | 0.043 | 0.024 | | |

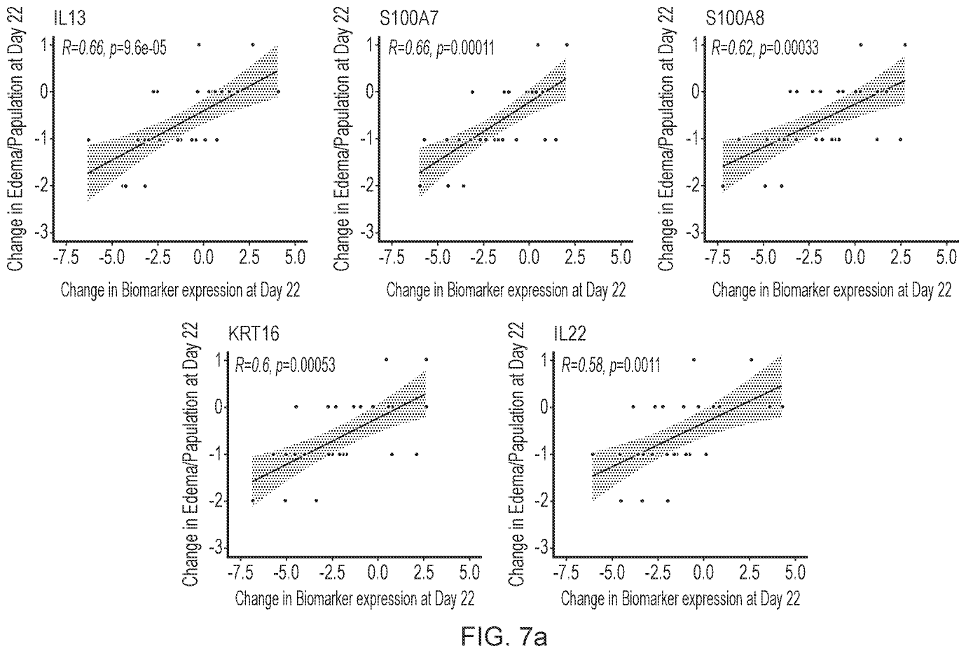

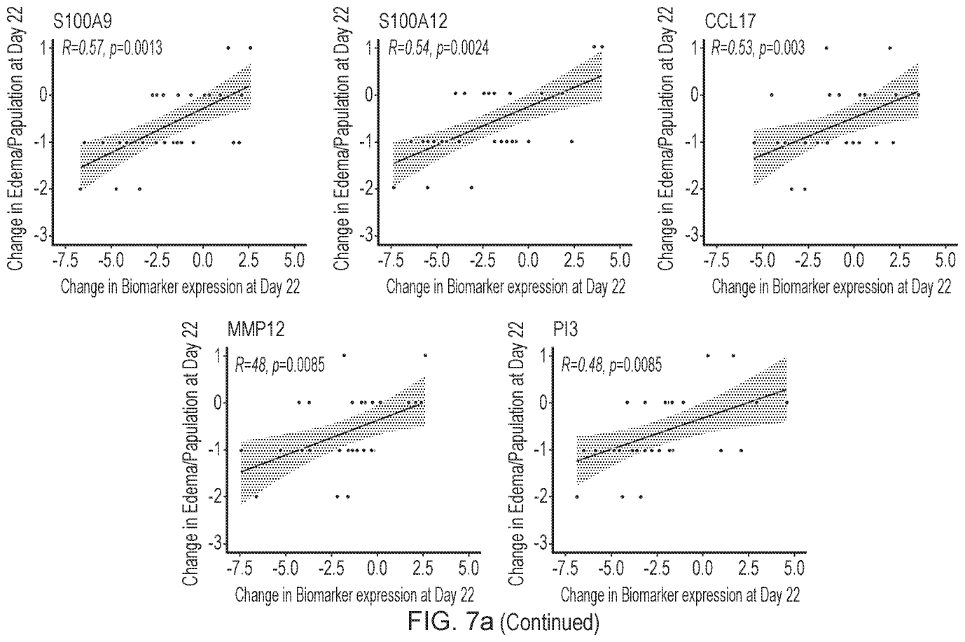

FIGS. 7a and 7b show the change in expression of biomarkers (IL13, S100A7, S100A8, KRT16, IL22, S100A9, S100A12, CCL17, MMP12, PI3, CCL22, DEFB4A/DEFB4B, IL19 and LOR) that were found to correlate with edema/papulation. The graphs show the biomarker change at Day 22 compared to baseline. Biomarkers of Th2 pathway, Th17/Th22 pathways and proliferation show highest correlation for the edema/papulation response. LOR as epithelia barrier marker correlates negatively.

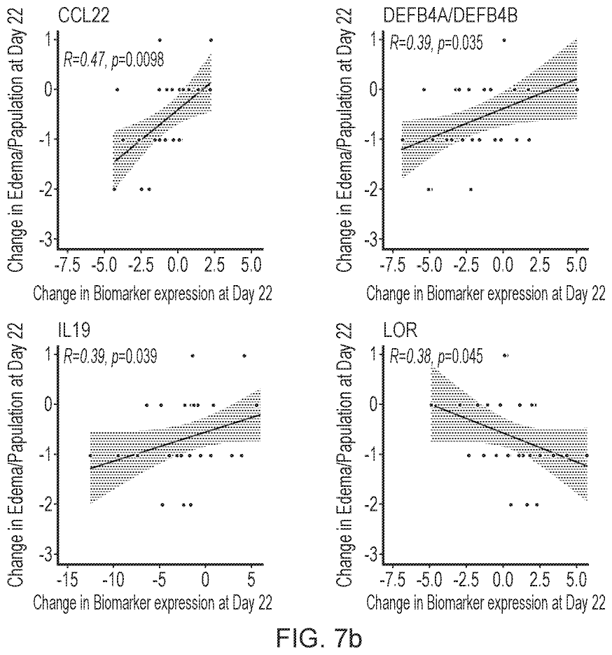

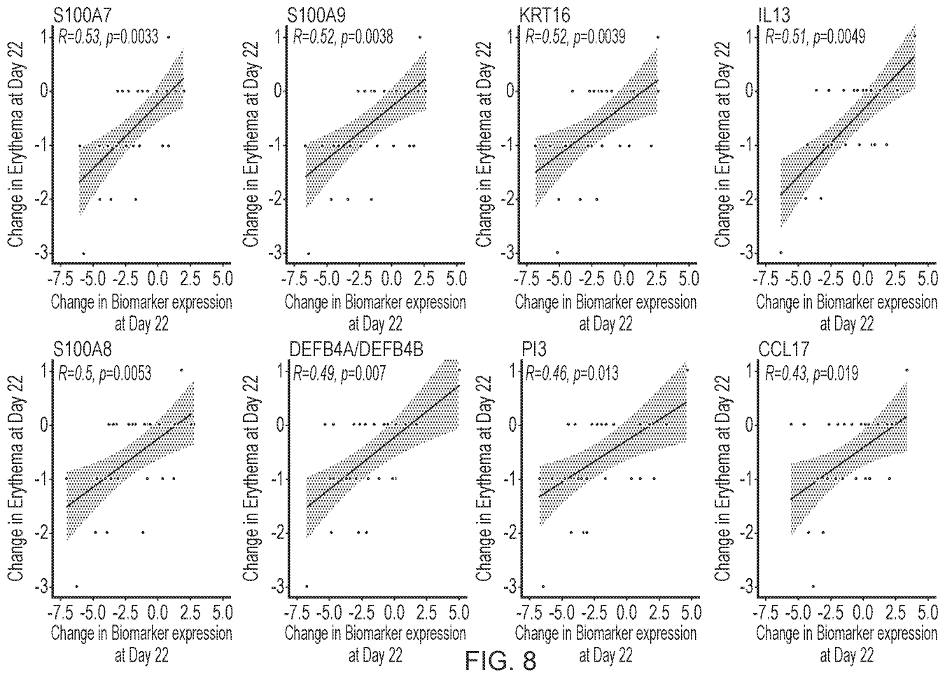

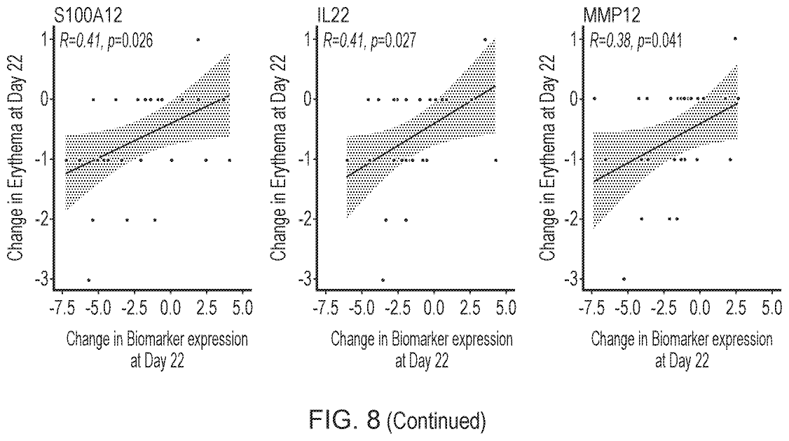

FIG. 8 shows the change in expression of biomarkers (S100A7, S100A9, KRT16, IL13, S100A8, DEFB4A/DEFB4B, PI3, CCL17, S100A12, IL22 and MMP12) that were found to correlate with erythema. The graphs show the biomarker change at Day 22 compared to baseline. Biomarkers of Th17/22 pathway, proliferation and TH2 pathway show highest correlation.

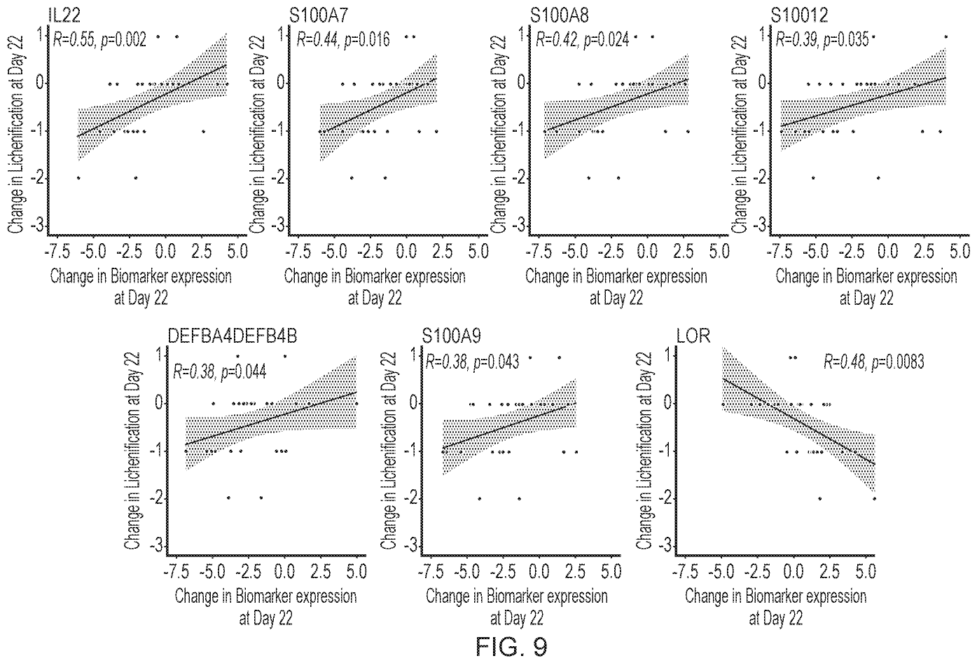

FIG. 9 shows the change in expression of biomarkers (IL22, S100A7, S100A8, S100A12, DEFB4A/DEFB4B, S100A9 and LOR) that were found to correlate with lichenification. The graphs show the biomarker change at Day 22 compared to baseline. Biomarkers of Th2 pathway, Th17/Th22, skin barrier/terminal differentiation show highest correlation.

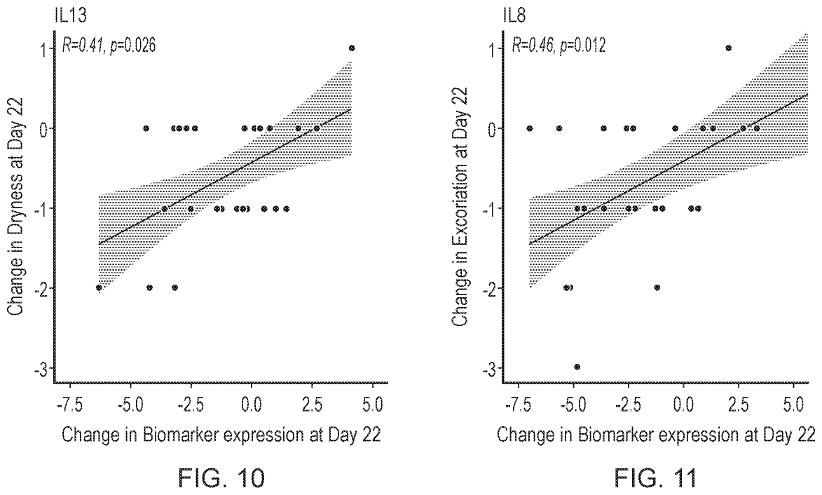

FIG. 10 shows the change expression of biomarkers (IL13) that were found to correlate with dryness. The graphs show the biomarker change at Day 22 compared to baseline. Biomarkers of Th2 and Th17 pathway show highest correlation.

FIG. 11 shows the change in expression of biomarkers (IL8) that were found to correlate with excoriation. The graphs show the biomarker change at Day 22 compared to baseline.

### Example 3: In Vitro Model for Evaluation of Anti-Inflammatory Effects

Modulation of immune mediators by a particular compound can be assessed using cell-based assays. Freshly isolated human peripheral blood mononuclear cells are seeded in the presence of plate bound anti-CD3, soluble anti-CD28 or phytohemagglutinin for 48 hours with the test compound. Cytokine and chemokine release is assessed using ELISA assays.

1. A halogenated salicylanilide selected from niclosamide and oxyclozanide, or a pharmaceutically acceptable salt or hydrate thereof, for use in topical anti-inflammatory treatment of one or more clinical signs or symptoms of an inflammatory skin condition in a subject, wherein the one or more clinical signs or symptoms are associated with an abnormal inflammatory response, and the topical anti-inflammatory treatment modulates expression in lesional skin of one or more immune effectors selected from proinflammatory mediators and skin barrier molecules.

2. The halogenated salicylanilide, or a pharmaceutically acceptable salt or hydrate thereof, for the use of claim 1, wherein the one or more clinical signs or symptoms are associated with skin barrier dysfunction.

3. The halogenated salicylanilide, or a pharmaceutically acceptable salt or hydrate thereof, for the use of claim 1 or claim 2, wherein the topical anti-inflammatory treatment decreases expression in lesional skin of one or more proinflammatory mediators.

4. The halogenated salicylanilide, or a pharmaceutically acceptable salt or hydrate thereof, for the use of any one of the preceding claims, wherein the topical anti-inflammatory treatment increases expression in lesional skin of one or more skin barrier molecules.

5. The halogenated salicylanilide, or a pharmaceutically acceptable salt or hydrate thereof, for the use of any one of the preceding claims, wherein the one or more clinical signs or symptoms are selected from erythema, excoriation, lichenification, edema, papulation, dryness, pruritus, scaling, oozing and crusting.

6. The halogenated salicylanilide, or a pharmaceutically acceptable salt or hydrate thereof, for the use of any one of the preceding claims, wherein the one or more clinical signs or symptoms is selected from erythema, excoriation, lichenification, edema, papulation and dryness.

7. The halogenated salicylanilide, or a pharmaceutically acceptable salt or hydrate thereof, for the use of any one of the preceding claims, wherein the one or more clinical signs or symptoms is selected from erythema, lichenification, edema and papulation.

8. The halogenated salicylanilide, or a pharmaceutically acceptable salt or hydrate thereof, for the use of any one of the preceding claims, wherein the one or more clinical signs or symptoms are associated with an abnormal (such as elevated) level of one or more proinflammatory mediators in lesional skin and the topical anti-inflammatory treatment reduces the abnormal level of said one or more proinflammatory mediators.

9. The halogenated salicylanilide, or a pharmaceutically acceptable salt or hydrate thereof, for the use of any one of the preceding claims, wherein the one or more clinical signs or symptoms are associated with skin barrier dysfunction including a deficiency in one or more skin barrier molecules and the topical anti-inflammatory treatment increases expression in lesional skin of one or more skin barrier molecules.

10. A halogenated salicylanilide selected from niclosamide and oxyclozanide, or a pharmaceutically acceptable salt or hydrate thereof, for use in modulating an abnormal inflammatory response associated with an inflammatory skin condition in a subject by topically applying the halogenated salicylanilide, or a pharmaceutically acceptable salt or hydrate thereof, to the skin of the subject, wherein the abnormal inflammatory response is modulated by modulating expression in lesional skin of one or more immune effectors selected from proinflammatory mediators and skin barrier molecules, thereby decreasing expression in lesional skin of one or more proinflammatory mediators, and increasing expression in lesional skin of one or more skin barrier molecules.

11. A halogenated salicylanilide selected from niclosamide and oxyclozanide, or a pharmaceutically acceptable salt or hydrate thereof, for use in improving skin barrier function associated with an inflammatory skin condition in a subject by topically applying the halogenated salicylanilide, or a pharmaceutically acceptable salt or hydrate thereof, to the skin of the subject, wherein the skin barrier function is improved by modulating expression in lesional skin of one or more immune effectors selected from proinflammatory mediators and skin barrier molecules.

12. The halogenated salicylanilide, or a pharmaceutically acceptable salt or hydrate thereof, for the use of claim 11, wherein decreased expression in lesional skin of one or more proinflammatory mediators is provided.

13. The halogenated salicylanilide, or a pharmaceutically acceptable salt or hydrate thereof, for the use of claim 11 or claim 12, wherein increased expression in lesional skin of one or more skin barrier molecules is provided.

14. The halogenated salicylanilide, or a pharmaceutically acceptable salt or hydrate thereof, for the use of any one of the preceding claims, wherein the proinflammatory mediators are selected from cytokines, enzymes, antibacterial proteins and peptides, and immune cells.

15. The halogenated salicylanilide, or a pharmaceutically acceptable salt or hydrate thereof, for the use of any one of the preceding claims, wherein the proinflammatory mediators selected from proinflammatory cytokines, proinflammatory enzymes and immune cells.

16. The halogenated salicylanilide, or a pharmaceutically acceptable salt or hydrate thereof, for the use of any one of the preceding claims, wherein the skin barrier molecules are selected from structural skin barrier proteins (e.g. LOR, FLG, DGAT2, FAXDC2) and skin barrier lipids (e.g. ACOX2, EVOLV3, FA2H, FAR2, KRT79, PNPLA3).

17. The halogenated salicylanilide, or a pharmaceutically acceptable salt or hydrate thereof, for the use of any one of the preceding claims, wherein the abnormal inflammatory response involves a Th1, Th2, Th17 and/or Th22-type inflammatory response.

18. The halogenated salicylanilide, or a pharmaceutically acceptable salt or hydrate thereof, for the use of any one of the preceding claims, wherein the expression in lesional skin of said one or more immune effectors are associated with activation of Th1, Th2, Th17 and/or Th22 cells.

19. The halogenated salicylanilide, or a pharmaceutically acceptable salt or hydrate thereof, for the use of any one of the preceding claims, wherein the expression of the one or more immune effectors is modulated by attenuating one or more responses selected from Th1, Th2, Th17 and Th22-type inflammatory response(s).

20. The halogenated salicylanilide, or a pharmaceutically acceptable salt or hydrate thereof, for the use of any one of the preceding claims, wherein the one or more immune effectors are selected from S100A12, S100A9, PI3, CXCL1, KRT16, MMP12, IL13, CCL17, CCL22, IL8, S100A8, S100A7, IL22, IL17A, IL19, CAMP, DEFB4A/DEFB4B, LOR, IL1B, IL6, IL17C, IL15, IL15RA, FOXP3, FLG, CXCL10, CCL20, CXCL2, IL12B, IL23A, CCL18, IL10, IL5, TSLPR, CD86, CCL19, IL24, ANXA6, SPTLC3, CCR7, CD2, CD28, CD3D, CD3G, CCL2, CCR1, CCR2, IFNGR2, IL12RB2, IL2RA, IRF1, CCR6, IL6R, LCN2, STAT3, IL37, TNFSF4, S100P, SERPINB1, SERPINB4, CCL13, CCR5, IL4R, IL7R, IL1F10, CDSN, CERS3, CLN8, ELOVL3, EREG, FA2H, FAR2, KRT79, PNPLA3, PPL, TJP3, ACER1, ANXA9, CLDN1, CLDN23, DGAT2, DHCR7, FAXDC2, KRT23, KRT77, SCEL, ACOX2 and ACSL1.

21. The halogenated salicylanilide, or a pharmaceutically acceptable salt or hydrate thereof, for the use of any one of the preceding claims, wherein the one or more immune effectors are selected from S100A12, S100A9, PI3, CXCL1, S100A7, IL17C, CCL20, CCL18, IL10, SPTLC3, CCL2, CCR1, IFNGR2, CCR6, LCN2, STAT3, TNFSF4, CCL13, IL4R, IL1F10, ELOVL3, FA2H, FAR2, KRT79, PNPLA3, DGAT2, FAXDC2 and ACOX2.

22. The halogenated salicylanilide, or a pharmaceutically acceptable salt or hydrate thereof, for the use of any one of the preceding claims, wherein the one or more immune effectors are selected from S100A12, S100A9, PI3, CXCL1, S100A7, IL17C, CCL20, CCL18, IL10, SPTLC3, CCL2, CCR1, IFNGR2, CCR6, LCN2, STAT3, TNFSF4, CCL13, IL4R and IL1F10.

23. The halogenated salicylanilide, or a pharmaceutically acceptable salt or hydrate thereof, for the use of any one of the preceding claims, wherein the one or more immune effectors are selected from S100A12, S100A9, S100A7, PI3, CXCL1, LOR, FLG, CDSN, CERS3, CLN8, ELOVL3, EREG, FA2H, FAR2, KRT79, PNPLA3, PPL, TJP3, ACER1, ANXA9, CLDN1, CLDN23, DGAT2, DHCR7, FAXDC2, KRT23, KRT77, ACOX2, ACSL1 and SCEL.

24. The halogenated salicylanilide, or a pharmaceutically acceptable salt or hydrate thereof, for the use of any one of the preceding claims, wherein the one or more immune effectors are selected from S100A12, S100A9, S100A7, PI3, CXCL1, ELOVL3, FA2H, FAR2, KRT79, PNPLA3, DGAT2, FAXDC2 and ACOX2.

25. The halogenated salicylanilide, or a pharmaceutically acceptable salt or hydrate thereof, for the use of any one of the preceding claims, wherein the one or more immune effectors are selected from S100A12, S100A9, S100A7, PI3 and CXCL1.

26. The halogenated salicylanilide, or a pharmaceutically acceptable salt or hydrate thereof, for the use of any one of claims 1-19, wherein the one or more immune effectors are selected from ELOVL3, FA2H, FAR2, KRT79, PNPLA3, DGAT2, FAXDC2 and ACOX2.

27. The halogenated salicylanilide, or a pharmaceutically acceptable salt or hydrate thereof, for the use of any one of the preceding claims, wherein the one or more immune effectors are selected from CD11c, FceR1 Epidermis and CD207 cells.

28. The halogenated salicylanilide, or a pharmaceutically acceptable salt or hydrate thereof, for the use of any one of the preceding claims, wherein the one or more immune effectors are CD11c Dermis cells.

29. The halogenated salicylanilide, or a pharmaceutically acceptable salt or hydrate thereof, for the use of any one of the preceding claims, wherein topical application of the halogenated salicylanilide, or a pharmaceutically acceptable salt or hydrate thereof, reduces or eliminates one or more clinical signs or symptoms selected from erythema, edema, papulation, lichenification and dryness, particularly one or more clinical signs or symptoms selected from erythema, edema, papulation and lichenification.

30. The halogenated salicylanilide, or a pharmaceutically acceptable salt or hydrate thereof, for the use of any one of the preceding claims, wherein topical application of the halogenated salicylanilide, or a pharmaceutically acceptable salt or hydrate thereof, modulates expression in lesional skin of one or more immune effectors selected from S100A7, S100A9, PI3, KRT16, IL13, S100A8, DEFB4A/DEFB4B, CCL17, S100A12, IL22, and MMP12, and the topical application reduces or eliminates erythema associated with the inflammatory skin condition.

31. The halogenated salicylanilide, or a pharmaceutically acceptable salt or hydrate thereof, for the use of any one of the preceding claims, wherein topical application of the halogenated salicylanilide, or a pharmaceutically acceptable salt or hydrate thereof, suppresses expression in lesional skin of one or more immune effectors selected from S100A7, S100A9, PI3 and S100A12, and the topical application reduces or eliminates erythema associated with the inflammatory skin condition.

32. The halogenated salicylanilide, or a pharmaceutically acceptable salt or hydrate thereof, for the use of any one of the preceding claims, wherein topical application of the halogenated salicylanilide, or a pharmaceutically acceptable salt or hydrate thereof, modulates expression in lesional skin of IL8, and the topical application reduces or eliminates excoriation associated with the inflammatory skin condition.

33. The halogenated salicylanilide, or a pharmaceutically acceptable salt or hydrate thereof, for the use of any one of the preceding claims, wherein topical application of the halogenated salicylanilide, or a pharmaceutically acceptable salt or hydrate thereof, modulates expression in lesional skin of one or more immune effectors selected from IL22, S100A9, S100A8, S100A12, S100A7, DEFB4A/DEFB4B, and LOR, and the topical application reduces or eliminates lichenification associated with the inflammatory skin condition.

34. The halogenated salicylanilide, or a pharmaceutically acceptable salt or hydrate thereof, for the use of any one of the preceding claims, wherein topical application of the halogenated salicylanilide, or a pharmaceutically acceptable salt or hydrate thereof, suppresses expression in lesional skin of one or more immune effectors selected from S100A9, S100A12 and S100A7, and the topical application reduces or eliminates lichenification associated with the inflammatory skin condition.

35. The halogenated salicylanilide, or a pharmaceutically acceptable salt or hydrate thereof, for the use of any one of the preceding claims, wherein topical application of the halogenated salicylanilide, or a pharmaceutically acceptable salt or hydrate thereof, modulates expression in lesional skin of one or more immune effectors selected S100A12, S100A9, PI3, KRT16, MMP12, IL13, CCL17, CCL22, S100A8, S100A7, IL22, IL19, DEFB4A/DEFB4B and LOR, and the topical application reduces or eliminates edema and/or papulation associated with the inflammatory skin condition.

36. The halogenated salicylanilide, or a pharmaceutically acceptable salt or hydrate thereof, for the use of any one of the preceding claims, wherein topical application of the halogenated salicylanilide, or a pharmaceutically acceptable salt or hydrate thereof, suppresses expression in lesional skin of one or more immune effectors selected S100A12, S100A9 and PI3, and the topical application reduces or eliminates edema and/or papulation associated with the inflammatory skin condition.

37. The halogenated salicylanilide, or a pharmaceutically acceptable salt or hydrate thereof, for the use of any one of the preceding claims, wherein topical application of the halogenated salicylanilide, or a pharmaceutically acceptable salt or hydrate thereof, modulates expression in lesional skin of IL13, and the topical application reduces or eliminates dryness associated with the inflammatory skin condition.

38. A halogenated salicylanilide selected from niclosamide and oxyclozanide, or a pharmaceutically acceptable salt or hydrate thereof, for use in topical anti-inflammatory treatment of erythema associated with an inflammatory skin condition in a subject having abnormal (such as elevated) level of one or more immune effectors selected from S100A7, S100A9, PI3, KRT16, IL13, S100A8, DEFB4A/DEFB4B, CCL17, S100A12, IL22, and MMP12 in lesional skin.

39. The halogenated salicylanilide, or a pharmaceutically acceptable salt or hydrate thereof, for the use of claim 38, wherein the subject has abnormal (such as elevated) level of one or more immune effectors selected from S100A7, S100A9, PI3 and S100A12.

40. The halogenated salicylanilide, or a pharmaceutically acceptable salt or hydrate thereof, for the use of claim 38 or claim 39, wherein the topical anti-inflammatory treatment suppresses expression in lesional skin of one or more immune effectors selected from S100A7, S100A9, PI3, KRT16, IL13, S100A8, DEFB4A/DEFB4B, CCL17, S100A12, IL22, and MMP12.

41. The halogenated salicylanilide, or a pharmaceutically acceptable salt or hydrate thereof, for the use of claim 38 or claim 39, wherein the topical anti-inflammatory treatment suppresses expression in lesional skin of one or more immune effectors selected from S100A7, S100A9, PI3, and S100A12.

42. A halogenated salicylanilide selected from niclosamide and oxyclozanide, or a pharmaceutically acceptable salt or hydrate thereof, for use in topical anti-inflammatory treatment of excoriation associated with an inflammatory skin condition in a subject having abnormal (such as elevated) level of IL8 in lesional skin.

43. The halogenated salicylanilide, or a pharmaceutically acceptable salt or hydrate thereof, for the use of claim 42, wherein the topical anti-inflammatory treatment suppresses expression in lesional skin of IL8.

44. A halogenated salicylanilide selected from niclosamide and oxyclozanide, or a pharmaceutically acceptable salt or hydrate thereof, for use in topical anti-inflammatory treatment of lichenification associated with an inflammatory skin condition in a subject having abnormal (such as elevated) level of one or more immune effectors selected from IL22, S100A9, S100A8, S100A12, S100A7, and DEFB4A/DEFB4B in lesional skin.

45. The halogenated salicylanilide, or a pharmaceutically acceptable salt or hydrate thereof, for the use of claim 44, wherein the subject has abnormal (such as elevated) level of one or more immune effectors selected from S100A9, S100A12 and S100A7.

46. The halogenated salicylanilide, or a pharmaceutically acceptable salt or hydrate thereof, for the use of claim 44 or claim 45, wherein the subject has a deficiency in LOR and the topical anti-inflammatory treatment promotes expression in lesional skin of LOR.

47. The halogenated salicylanilide, or a pharmaceutically acceptable salt or hydrate thereof, for the use of any one of claims 44-46, wherein the topical anti-inflammatory treatment suppresses expression in lesional skin of one or more immune effectors selected from IL22, S100A9, S100A8, S100A7, S100A12, IL19, and DEFB4A/DEFB4B.

48. The halogenated salicylanilide, or a pharmaceutically acceptable salt or hydrate thereof, for the use of any one of claims 44-46, wherein the topical anti-inflammatory treatment suppresses expression in lesional skin of one or more immune effectors selected from S100A9, S100A8 and S100A7.

49. A halogenated salicylanilide selected from niclosamide and oxyclozanide, or a pharmaceutically acceptable salt or hydrate thereof, for use in topical anti-inflammatory treatment of edema and/or papulation associated with an inflammatory skin condition in a subject having abnormal (such as elevated) level of one or more immune effectors selected from S100A12, S100A9, PI3, KRT16, MMP12, IL13, CCL17, CCL22, S100A8, S100A7, IL19, IL22 and DEFB4A/DEFB4B.

50. The halogenated salicylanilide, or a pharmaceutically acceptable salt or hydrate thereof, for the use of claim 49, wherein the subject has abnormal (such as elevated) level of one or more immune effectors selected from S100A12, S100A9 and PI3.

51. The halogenated salicylanilide, or a pharmaceutically acceptable salt or hydrate thereof, for the use of claim 49 or claim 50, wherein the subject has a deficiency in LOR and the topical anti-inflammatory treatment promotes expression in lesional skin of LOR.

52. The halogenated salicylanilide, or a pharmaceutically acceptable salt or hydrate thereof, for the use of any one of claims 49-51, wherein the topical anti-inflammatory treatment suppresses expression in lesional skin of one or more immune effectors selected S100A12, S100A9, PI3, KRT16, MMP12, IL13, CCL17, CCL22, S100A8, S100A7, IL22, IL19 and DEFB4A/DEFB4B.

53. The halogenated salicylanilide, or a pharmaceutically acceptable salt or hydrate thereof, for the use of any one of claims 49-51, wherein the topical anti-inflammatory treatment suppresses expression in lesional skin of one or more immune effectors selected S100A12, S100A9 and PI3.

54. A halogenated salicylanilide selected from niclosamide and oxyclozanide, or a pharmaceutically acceptable salt or hydrate thereof, for use in topical anti-inflammatory treatment of dryness associated with an inflammatory skin condition in a subject having abnormal (such as elevated) level of IL13 in lesional skin.

55. The halogenated salicylanilide, or a pharmaceutic ally acceptable salt or hydrate thereof, for the use of claim 54, wherein the topical anti-inflammatory treatment suppresses expression in lesional skin of IL13.

56. The halogenated salicylanilide, or a pharmaceutically acceptable salt or hydrate thereof, for the use of any one of the preceding claims, wherein the inflammatory skin condition is a clinically uninfected inflammatory skin condition, such as a clinically uninfected atopic dermatitis.

57. The halogenated salicylanilide, or a pharmaceutically acceptable salt or hydrate thereof, for the use of any one of the preceding claims, wherein the inflammatory skin condition is selected from psoriasis, dermatitis, scleroderma, disorders of hair follicles and sebaceous glands, acne, rosacea, rhinophyma, cutaneous lupus, inflammatory skin reactions (for example drug eruptions, erythema multiforme, erythema nodosum, and granuloma annulare), skin inflammation associated with fungal or yeast infections (e.g. dermatophytosis), urticaria, dermatitis herpetiformis, lichen planus, hidradenitis suppurativa, pitayriasis rosea, chronic sinusitis, chronic rhinosinusitis, lupus, vitiligo and keratosis pilaris.

58. The halogenated salicylanilide, or a pharmaceutically acceptable salt or hydrate thereof, for the use of any one of the preceding claims, wherein the inflammatory skin condition is not acne.

59. The halogenated salicylanilide, or a pharmaceutically acceptable salt or hydrate thereof, for the use of any one of the preceding claims, wherein the inflammatory skin condition is selected from psoriasis, rosacea and dermatitis.

60. The halogenated salicylanilide, or a pharmaceutically acceptable salt or hydrate thereof, for the use of any one of the preceding claims, wherein the inflammatory skin condition is dermatitis.

61. A halogenated salicylanilide selected from niclosamide and oxyclozanide, or a pharmaceutically acceptable salt or hydrate thereof, for the use in topical anti-inflammatory treatment of one or more clinical signs or symptoms of dermatitis (e.g. atopic dermatitis) in a subject, wherein the one or more clinical signs or symptoms are selected from erythema, excoriation, lichenification, edema, papulation and dryness, particularly erythema, lichenification, edema and papulation.

62. The halogenated salicylanilide, or a pharmaceutically acceptable salt or hydrate thereof, for the use of any one of the preceding claims, wherein the inflammatory skin condition is dermatitis selected from contact dermatitis, allergic contact dermatitis, irritant contact dermatitis, atopic dermatitis, seborrhoeic dermatitis, actinic dermatitis, hand and foot dermatitis, pompholyx dermatitis, lichen simplex chronicus (neurodermatitis), exfoliative dermatitis (erythroderma), asteatotic dermatitis, carcinomatous dermatitis, nummular dermatitis, neonatal dermatitis, paediatric dermatitis, diaper dermatitis, stasis dermatitis, perioral dermatitis, dermatomyositis, eczematous dermatitis, photoallergic dermatitis, phototoxic dermatitis, phytophotodermatitis and radiation-induced dermatitis.

63. The halogenated salicylanilide, or a pharmaceutically acceptable salt or hydrate thereof, for the use of any one of the preceding claims, wherein the dermatitis is mild to moderate dermatitis.

64. The halogenated salicylanilide, or a pharmaceutically acceptable salt or hydrate thereof, for the use of any one of the preceding claims, wherein the dermatitis is moderate dermatitis.

65. The halogenated salicylanilide, or a pharmaceutically acceptable salt or hydrate thereof, for the use of any one of the preceding claims, wherein the dermatitis is mild dermatitis.

66. The halogenated salicylanilide, or a pharmaceutically acceptable salt or hydrate thereof, for the use of any one of the preceding claims, wherein the dermatitis is moderate to severe dermatitis.

67. The halogenated salicylanilide, or a pharmaceutically acceptable salt or hydrate thereof, for the use of any one of the preceding claims, wherein the dermatitis is severe dermatitis.

68. The halogenated salicylanilide, or a pharmaceutically acceptable salt or hydrate thereof, for the use of any one of the preceding claims, for further use in the treatment of an exacerbation of dermatitis.

69. The halogenated salicylanilide, or a pharmaceutically acceptable salt or hydrate thereof, for the use of any one of the preceding claims, wherein the dermatitis is an acute form of dermatitis.

70. The halogenated salicylanilide, or a pharmaceutically acceptable salt or hydrate thereof, for the use of any one of the preceding claims, wherein the dermatitis is a chronic form of dermatitis.

71. The halogenated salicylanilide, or a pharmaceutically acceptable salt or hydrate thereof, for the use of any one of the preceding claims, wherein the inflammatory skin condition is atopic dermatitis.

72. The halogenated salicylanilide, or a pharmaceutically acceptable salt or hydrate thereof, for the use of any one of the preceding claims, wherein the inflammatory skin condition is atopic dermatitis and topical application of the halogenated salicylanilide, or a pharmaceutically acceptable salt or hydrate thereof, reduces the Total Sign Score (TSS) of atopic dermatitis by more than 10%, such as at least 20%.

73. A halogenated salicylanilide selected from niclosamide and oxyclozanide, or a pharmaceutically acceptable salt or hydrate thereof, for the use in topical anti-inflammatory treatment of one or more clinical signs or symptoms of rosacea in a subject, wherein the one or more clinical signs or symptoms are selected from erythema, excoriation, lichenification, edema, papulation and dryness.

74. A halogenated salicylanilide selected from niclosamide and oxyclozanide, or a pharmaceutically acceptable salt or hydrate thereof, for the use in topical anti-inflammatory treatment of one or more clinical signs or symptoms of psoriasis in a subject, wherein the one or more clinical signs or symptoms are selected from erythema, excoriation, lichenification, edema, papulation and dryness.

75. The halogenated salicylanilide, or a pharmaceutically acceptable salt or hydrate thereof, for the use of any one of the preceding claims, wherein the one or more clinical signs or symptoms are associated with a Th1, Th2, Th17 and/or Th22-type inflammatory response.

76. The halogenated salicylanilide, or a pharmaceutically acceptable salt or hydrate thereof, for the use of any one of the preceding claims, wherein the halogenated salicylanilide, or a pharmaceutically acceptable salt or hydrate thereof, is topically applied to inflammatory lesional skin of the subject.

77. The halogenated salicylanilide, or a pharmaceutically acceptable salt or hydrate thereof, for the use of any one of the preceding claims, wherein a therapeutically effective amount of the halogenated salicylanilide, or a pharmaceutically acceptable salt or hydrate thereof, is topically applied at least once daily for more than 5 days, such as more than 10 days, more than 15 days more than 20 days, more than 25 days or more than 30 days, to the skin of the subject.

78. The halogenated salicylanilide, or a pharmaceutically acceptable salt or hydrate thereof, for the use of any one of the preceding claims, wherein the halogenated salicylanilide, or a pharmaceutically acceptable salt or hydrate thereof, is topically administered in the form a topical composition selected from a topical cream, ointment, gel, paste, lotion, foam, suspension and solution, particularly a topical cream or a topical gel.

79. The halogenated salicylanilide, or a pharmaceutically acceptable salt or hydrate thereof, for the use of any one of the preceding claims, wherein the topical composition comprises the halogenated salicylanilide, or a pharmaceutically acceptable salt or hydrate thereof, and a formulation base selected from an oleaginous base (e.g. petrolatum, white petrolatum, yellow ointment or white ointment), an absorption base (e.g. hydrophilic petrolatum or lanolin), a water-removable base (oil in water emulsion); and a water-soluble base (e.g. a polyethylene glycol).

80. The halogenated salicylanilide, or a pharmaceutically acceptable salt or hydrate thereof, for the use of any one of the preceding claims, wherein the topical composition is a non-aqueous composition, particularly a non-aqueous topical cream or a topical gel.

81. The halogenated salicylanilide, or a pharmaceutically acceptable salt or hydrate thereof, for the use of any one of the preceding claims, wherein the topical composition is an aqueous composition.

82. The halogenated salicylanilide, or a pharmaceutically acceptable salt or hydrate thereof, for the use of any one of the preceding claims, wherein the topical composition is a non-aqueous topical composition comprising:
(i) the halogenated salicylanilide, or a pharmaceutically acceptable salt or hydrate thereof; and
(ii) polyethylene glycol (PEG), preferably a PEG with a melting point of less than 40° C.

83. The halogenated salicylanilide, or a pharmaceutically acceptable salt or hydrate thereof, for the use of any one of the preceding claims, wherein the topical composition is a non-aqueous topical composition comprising:
(i) 0.01 to 4.5% (for example 0.1 to 3% or about 2%) by weight of the halogenated salicylanilide, or a pharmaceutically acceptable salt or hydrate thereof; and
(ii) at least 70% (for example at least 90%) by weight of a PEG, wherein the average molecular weight of the PEG is 600 or less (for example less than 600 or from about 200 to about 600 or about 400).

84. The halogenated salicylanilide, or a pharmaceutically acceptable salt or hydrate thereof, for the use of any one of the preceding claims, wherein the topical composition further comprises a non-polymeric glycol (e.g. propylene glycol).

85. The halogenated salicylanilide, or a pharmaceutically acceptable salt or hydrate thereof, for the use of any one of the preceding claims, wherein the halogenated salicylanilide, or a pharmaceutically acceptable salt or hydrate thereof, is dissolved or partially dissolved in the topical composition.

86. The halogenated salicylanilide, or a pharmaceutically acceptable salt or hydrate thereof, for the use of any one of the preceding claims, wherein the inflammatory skin condition is atopic dermatitis and the subject has a condition selected from asthma, rhinitis and a food allergy in addition to atopic dermatitis.

87. A halogenated salicylanilide selected from niclosamide and oxyclozanide, or a pharmaceutically acceptable salt or hydrate thereof, for use in topical anti-inflammatory treatment of one or more clinical signs or symptoms of an inflammatory ocular condition in a subject, wherein the one or more clinical signs or symptoms are associated with an abnormal inflammatory response, and the topical anti-inflammatory treatment modulates expression of one or more immune effectors selected from proinflammatory mediators and ocular surface epithelial barrier molecules.

88. The halogenated salicylanilide, or a pharmaceutically acceptable salt or hydrate thereof, for the use of claim 87, wherein the topical anti-inflammatory treatment provides decreased expression in ocular and ocular-associated tissue (e.g. cornea) and/or in pre-corneal tear film of one or more proinflammatory mediators.

89. The halogenated salicylanilide, or a pharmaceutically acceptable salt or hydrate thereof, for the use of claim 87 or claim 88, wherein the topical anti-inflammatory treatment provides increased expression in ocular and ocular-associated tissue (e.g. cornea) of one or more ocular surface epithelial barrier molecules.

90. The halogenated salicylanilide, or a pharmaceutically acceptable salt or hydrate thereof, for the use of any one of claims 87-89, wherein the one or more clinical signs or symptoms are associated with an abnormal (such as elevated) level of one or more proinflammatory mediators in ocular and ocular-associated tissue (e.g. cornea) and/or in pre-corneal tear film and the topical anti-inflammatory treatment reduces the abnormal level of said one or more proinflammatory mediators.

91. The halogenated salicylanilide, or a pharmaceutically acceptable salt or hydrate thereof, for the use of any one of claims 87-90, wherein the one or more clinical signs or symptoms are associated with deficiency in one or more ocular surface epithelial barrier molecules and the topical anti-inflammatory treatment provides increased expression in in ocular and ocular-associated tissue (e.g. cornea) of one or more ocular surface epithelial barrier molecules.

92. The halogenated salicylanilide, or a pharmaceutically acceptable salt or hydrate thereof, for the use of any one of claims 87-91, wherein the inflammatory ocular condition is dry eye disease (DED).

93. The halogenated salicylanilide, or a pharmaceutically acceptable salt or hydrate thereof, for the use of any one of claims 87-92, wherein the one or more immune effectors are selected from proinflammatory mediators.

94. The halogenated salicylanilide, or a pharmaceutically acceptable salt or hydrate thereof, for the use of any one of claims 87-93, wherein the proinflammatory mediators are selected from proinflammatory cytokines, proinflammatory enzymes, antibacterial proteins and peptides, and immune cells.

95. The halogenated salicylanilide, or a pharmaceutically acceptable salt or hydrate thereof, for the use of any one of claims 87-94, wherein the proinflammatory mediators selected from proinflammatory cytokines, proinflammatory enzymes and immune cells.

96. The halogenated salicylanilide, or a pharmaceutically acceptable salt or hydrate thereof, for the use of any one of claims 87-95, wherein the ocular surface epithelial barrier molecules are selected from structural ocular surface epithelial barrier proteins (e.g. LOR and FLG) and ocular surface epithelial barrier lipids.

97. The halogenated salicylanilide, or a pharmaceutically acceptable salt or hydrate thereof, for the use of any one of claims 87-96, wherein the abnormal inflammatory response involves a Th1, Th2, Th17 and/or Th22-type inflammatory response.

98. The halogenated salicylanilide, or a pharmaceutically acceptable salt or hydrate thereof, for the use of any one of claims 87-97, wherein the expression in ocular and ocular-associated tissue (e.g. cornea) and/or pre-corneal tear film of said one or more immune effectors are associated with activation of Th1, Th2, Th17 and/or Th22 cells.

99. The halogenated salicylanilide, or a pharmaceutically acceptable salt or hydrate thereof, for the use of any one of claims 87-98, wherein the expression of said one or more immune effectors is modulated by attenuating one or more responses selected from Th1, Th2, Th17 and Th22-type inflammatory response.

100. The halogenated salicylanilide, or a pharmaceutically acceptable salt or hydrate thereof, for the use of any one of claims 87-99, wherein the one or more immune effectors are selected from S100A12, S100A9, PI3, CXCL1, KRT16, MMP12, IL13, CCL17, CCL22, IL8, S100A8, S100A7, IL22, IL17A, IL19, CAMP, DEFB4A/DEFB4B, LOR, IL1B, IL6, IL17C, IL15, IL15RA, FOXP3, FLG, CXCL10, CCL20, CXCL2, IL12B, IL23A, CCL18, IL10, IL5, TSLPR, CD86, CCL19, IL24, ANXA6, SPTLC3, CCR7, CD2, CD28, CD3D, CD3G, CCL2, CCR1, CCR2, IFNGR2, IL12RB2, IL2RA, IRF1, CCR6, IL6R, LCN2, STAT3, IL37, TNFSF4, S100P, SERPINB1, SERPINB4, CCL13, CCR5, IL4R, IL7R, IL1F10, CDSN, CERS3, CLN8, ELOVL3, EREG, FA2H, FAR2, KRT79, PNPLA3, PPL, TJP3, ACER1, ANXA9, CLDN1, CLDN23, DGAT2, DHCR7, FAXDC2, KRT23, KRT77, SCEL, ACOX2 and ACSL1.

101. The halogenated salicylanilide, or a pharmaceutically acceptable salt or hydrate thereof, for the use of any one of claims 87-100, wherein the one or more immune effectors are selected from S100A12, S100A9, PI3, CXCL1, S100A7, IL17C, CCL20, CCL18, IL10, SPTLC3, CCL2, CCR1, IFNGR2, CCR6, LCN2, STAT3, TNFSF4, CCL13, IL4R, IL1F10, ELOVL3, FA2H, FAR2, KRT79, PNPLA3, DGAT2, FAXDC2 and ACOX2.

102. The halogenated salicylanilide, or a pharmaceutically acceptable salt or hydrate thereof, for the use of any one of claims 87-101, wherein the one or more immune effectors are selected from S100A12, S100A9, PI3, CXCL1, S100A7, IL17C, CCL20, CCL18, IL10, SPTLC3, CCL2, CCR1, IFNGR2, CCR6, LCN2, STAT3, TNFSF4, CCL13, IL4R and IL1F10,

103. The halogenated salicylanilide, or a pharmaceutically acceptable salt or hydrate thereof, for the use of any one of claims 87-102, wherein the one or more immune effectors are selected from S100A12, S100A9, S100A7, PI3, CXCL1, LOR, FLG, ELOVL3, FA2H, FAR2, KRT79, PNPLA3, DGAT2, FAXDC2 and ACOX2.

104. The halogenated salicylanilide, or a pharmaceutically acceptable salt or hydrate thereof, for the use of any one of claims 87-103, wherein the one or more immune effectors are selected from S100A12, S100A9, S100A7, PI3, CXCL1, LOR, FLG, CDSN, CERS3, CLN8, ELOVL3, EREG, FA2H, FAR2, KRT79, PNPLA3, PPL, TJP3, ACER1, ANXA9, CLDN1, CLDN23, DGAT2, DHCR7, FAXDC2, KRT23, KRT77, ACOX2, ACSL1 and SCEL.

105. The halogenated salicylanilide, or a pharmaceutically acceptable salt or hydrate thereof, for the use of any one of claims 87-104, wherein the one or more immune effectors are selected from S100A12, S100A9, S100A7, PI3 and CXCL1.

106. The halogenated salicylanilide, or a pharmaceutically acceptable salt or hydrate thereof, for the use of any one of claims 87-99, wherein the one or more immune effectors are selected from ELOVL3, FA2H, FAR2, KRT79, PNPLA3, DGAT2, FAXDC2 and ACOX2.

107. A halogenated salicylanilide selected from niclosamide and oxyclozanide, or a pharmaceutically acceptable salt or hydrate thereof, for the use in topical anti-inflammatory treatment of one or more clinical signs or symptoms of dry eye disease (DED) in a subject.

108. The halogenated salicylanilide, or a pharmaceutically acceptable salt or hydrate thereof, for the use of any one of claims 87-107, wherein the halogenated salicylanilide, or a pharmaceutically acceptable salt or hydrate thereof, is topically administered in the form an ophthalmic composition, such as an ophthalmic cream, ointment, gel, paste, lotion, foam, suspension and solution.

109. The halogenated salicylanilide, or a pharmaceutically acceptable salt or hydrate thereof, for the use of any one of the preceding claims, wherein the subject is a human.

110. The halogenated salicylanilide, or a pharmaceutically acceptable salt or hydrate thereof, for the use of any one of the preceding claims, wherein the subject is an adult human.

111. The halogenated salicylanilide, or a pharmaceutically acceptable salt or hydrate thereof, for the use of any one of the preceding claims, wherein the subject is a paediatric human, for example a human less than 18 years old.

112. The halogenated salicylanilide for the use of any one of the preceding claims, wherein the halogenated salicylanilide is oxyclozanide, or a pharmaceutically acceptable salt or hydrate thereof.

113. The halogenated salicylanilide for the use of any one of the preceding claims, wherein the halogenated salicylanilide is niclosamide, or a pharmaceutically acceptable salt or hydrate thereof, particularly the halogenated salicylanilide is niclosamide.

114. A method for treating one or more clinical signs or symptoms of an inflammatory skin condition in a subject, wherein the one or more clinical signs or symptoms are associated with an abnormal inflammatory response, the method comprising topically administrating a therapeutically effective amount of a halogenated salicylanilide selected from niclosamide and oxyclozanide, or a pharmaceutically acceptable salt or hydrate thereof, to the skin of the subject.

115. The method of claim 114, wherein the one or more clinical signs or symptoms are associated with skin barrier dysfunction.

116. The method of claim 114 or claim 115, wherein decreased expression in lesional skin of one or more proinflammatory mediators is provided.

117. The method of any one of claims 114-116, wherein increased expression in lesional skin of one or more skin barrier molecules is provided.

118. The method of any one of the claims 114-117, wherein the one or more clinical signs or symptoms are selected from erythema, excoriation, lichenification, edema, papulation, dryness, pruritus, scaling, oozing and crusting.

119. The method of any one of claims 114-118, wherein the one or more clinical signs or symptoms is selected from erythema, excoriation, lichenification, edema, papulation and dryness, particularly the one or more clinical signs or symptoms is selected from erythema, lichenification, edema and papulation.

120. The method of any one of claims 114-119, wherein the one or more clinical signs or symptoms are associated with an abnormal (such as elevated) level of one or more proinflammatory mediators in lesional skin and the topical anti-inflammatory treatment reduces the abnormal level of said one or more proinflammatory mediators.

121. The method of any one of claims 114-120, wherein the one or more clinical signs or symptoms are associated with skin barrier dysfunction including a deficiency in one or more skin barrier molecules and the topical anti-inflammatory treatment increases expression in lesional skin of one or more skin barrier molecules.

122. A method for modulating an abnormal inflammatory response associated with an inflammatory skin condition in a subject, the method comprising topically administrating a therapeutically effective amount of a halogenated salicylanilide selected from niclosamide and oxyclozanide, or a pharmaceutically acceptable salt or hydrate thereof, to the skin of the subject, and thereby providing modulated expression in lesional skin of immune effectors selected from proinflammatory mediators and skin barrier molecules, including providing decreased expression in lesional skin of one or more proinflammatory mediators, and increased expression in lesional skin of one or more skin barrier molecules.

123. A method for improving skin barrier function associated with an inflammatory skin condition in a subject, the method comprising topically administrating a therapeutically effective amount of a halogenated salicylanilide selected from niclosamide and oxyclozanide, or a pharmaceutically acceptable salt or hydrate thereof, to the skin of the subject, and thereby providing modulated expression in lesional skin of one or more immune effectors selected from proinflammatory mediators and skin barrier molecules, such as providing decreased expression in lesional skin of one or more proinflammatory mediators and increased expression in lesional skin of one or more skin barrier molecules.

124. A method for treating erythema associated with an inflammatory skin condition in a subject having abnormal (such as elevated) level of one or more immune effectors selected from S100A7, S100A9, PI3, KRT16, IL13, S100A8, DEFB4A/DEFB4B, CCL17, S100A12, IL22, and MMP12 in lesional skin, the method comprising topically administrating a therapeutically effective amount of a halogenated salicylanilide selected from niclosamide and oxyclozanide, or a pharmaceutically acceptable salt or hydrate thereof, to the skin of the subject.

125. The method of claim 124, wherein the subject has abnormal (such as elevated) level of one or more immune effectors selected from S100A7, S100A9, PI3 and S100A12.

126. A method for treating excoriation associated with an inflammatory skin condition in a subject having abnormal (such as elevated) level of IL8 in lesional skin, the method comprising topically administrating a therapeutically effective amount of a halogenated salicylanilide selected from niclosamide and oxyclozanide, or a pharmaceutically acceptable salt or hydrate thereof, to the skin of the subject.

127. A method for treating lichenification associated with an inflammatory skin condition in a subject having abnormal (such as elevated) level of one or more immune effectors selected from IL22, S100A9, S100A8, S100A7, and DEFB4A/DEFB4B in lesional skin, the method comprising topically administrating a therapeutically effective amount of a halogenated salicylanilide selected from niclosamide and oxyclozanide, or a pharmaceutically acceptable salt or hydrate thereof, to the skin of the subject.

128. The method of claim 127, wherein the subject has abnormal (such as elevated) level of one or more immune effectors selected from S100A9, S100A12 and S100A7.

129. A method for treating edema and/or papulation associated with an inflammatory skin condition in a subject having abnormal (such as elevated) level of one or more immune effectors selected from S100A12, S100A9, PI3, KRT16, MMP12, IL13, CCL17, CCL22, S100A8, S100A7, IL22, IL19 and DEFB4A/DEFB4B in lesional skin, the method comprising topically administrating a therapeutically effective amount of a halogenated salicylanilide selected from niclosamide and oxyclozanide, or a pharmaceutically acceptable salt or hydrate thereof, to the skin of the subject.

130. The method of claim 129, wherein the subject has abnormal (such as elevated) level of one or more immune effectors selected from S100A12, S100A9 and PI3.

131. A method for treating dryness associated with an inflammatory skin condition in a subject having abnormal (such as elevated) level of IL13 in lesional skin, the method comprising topically administrating a therapeutically effective amount of a halogenated salicylanilide selected from niclosamide and oxyclozanide, or a pharmaceutically acceptable salt or hydrate thereof, to the skin of the subject.

132. A method of treating one or more clinical signs or symptoms of dermatitis (e.g. atopic dermatitis) in a subject, wherein the one or more clinical signs or symptoms are selected from erythema, excoriation, lichenification, edema, papulation and dryness, the method comprising topically administrating a therapeutically effective amount of a halogenated salicylanilide selected from niclosamide and oxyclozanide, or a pharmaceutically acceptable salt or hydrate thereof, to the skin of the subject.

133. A method of treating one or more clinical signs or symptoms of rosacea in a subject, wherein the one or more clinical signs or symptoms are selected from erythema, excoriation, lichenification, edema, papulation and dryness, the method comprising topically administrating a therapeutically effective amount of a halogenated salicylanilide selected from niclosamide and oxyclozanide, or a pharmaceutically acceptable salt or hydrate thereof, to the skin of the subject.

134. A method of treating one or more clinical signs or symptoms of psoriasis in a subject, wherein the one or more clinical signs or symptoms are selected from erythema, excoriation, lichenification, edema, papulation and dryness, the method comprising topically administrating a therapeutically effective amount of a halogenated salicylanilide selected from niclosamide and oxyclozanide, or a pharmaceutically acceptable salt or hydrate thereof, to the skin of the subject.

135. The method of any one of claims 132-134, wherein the one or more clinical signs or symptoms are selected from erythema, lichenification, edema and papulation.

136. A method of treating one or more clinical signs or symptoms of an inflammatory ocular condition in a subject, wherein the one or more clinical signs or symptoms are associated with an abnormal inflammatory response and pre-corneal tear film dysfunction, the method comprising topically administrating a therapeutically effective amount of a halogenated salicylanilide selected from niclosamide and oxyclozanide, or a pharmaceutically acceptable salt or hydrate thereof, to the eye of the subject, and thereby providing modulated expression of one or more immune effectors.

137. A method of treating one or more clinical signs or symptoms of dry eye disease in a subject, the method comprising topically administrating a therapeutically effective amount of a halogenated salicylanilide selected from niclosamide and oxyclozanide, or a pharmaceutically acceptable salt or hydrate thereof, to the eye of the subject.

138. The method of any one of claims 114-137, wherein the subject is a human.

139. The method of claims 114-138, wherein the subject is an adult human.

140. The method of claims 114-139, wherein the subject is a paediatric human, for example a human less than 18 years old.

141. The method of claims 114-140, wherein the halogenated salicylanilide is oxyclozanide, or a pharmaceutically acceptable salt or hydrate thereof.

142. The method of claims 114-140, wherein the halogenated salicylanilide is niclosamide, or a pharmaceutically acceptable salt or hydrate thereof, particularly the halogenated salicylanilide is niclosamide.
